(19) United States
(12) Patent Application Publication
(10) Pub. No.: US 2015/0352111 A1
(43) Pub. Date: Dec. 10, 2015

(54) Therapeutic Indications of Kinase Inhibitors

(71) Applicant: Lawrence S. ZISMAN, Slingerlands, NY (US)

(72) Inventor: Lawrence S Zisman, Slingerlands, NY (US)

(73) Assignee: Pulmokine, Inc., Slingerlands, NY (US)

(21) Appl. No.: 14/760,165

(22) PCT Filed: Jan. 9, 2014
(86) PCT No.: PCT/US14/10773

§ 371 Date: Jul. 9, 2015

Related U.S. Application Data
(60) Provisional application No. 61/751,217, filed on Jan. 10, 2013.

(51) Int. Cl. A61K 31/506; A61K 31/497; C07D 241/20; A61K 31/4965; C07D 401/14; A61K 45/06; A61K 31/455; C07D 239/42; A61K 31/505; A61K 9/00; C07D 403/12; C07D 401/12; C07D 213/82

(52) U.S. Cl. CPC A61K 31/506; C07D 401/12; A61K 31/497; C07D 241/20; A61K 31/4965; C07D 401/14; C07D 213/82; A61K 31/455; C07D 239/42; A61K 31/505; A61K 9/0078; C07D 403/12; A61K 45/06

(57) ABSTRACT

Disclosed herein are compounds, compositions, and methods for preventing and treating diseases associated with protein kinase activity. The therapeutic indications described herein relate to receptor tyrosine kinase (RTK) inhibition for the treatment or prevention of vascular conditions and proliferative disorders. The disclosure also relates to irreversible RTK inhibitors.

### CROSS-REFERENCE TO RELATED APPLICATIONS

This application claims priority to U.S. Provisional Application No. 61/751,217 filed Jan. 10, 2013, the entire contents of which are hereby incorporated by reference in its entirety.

### STATEMENT OF GOVERNMENT-SPONSORED RESEARCH

This invention was made with United States government support under Grant Number 1R43HL102946-01 awarded by the National Institute of Health. The United States government has certain rights in the invention.

### FIELD OF THE INVENTION

The present disclosure relates generally to the treatment and prevention of disease associated with protein kinase activity. In particular, the present technology relates to therapeutic indications of protein kinase inhibitors and methods for the treatment or prevention of vascular conditions, cancer, and other disorders.

### BACKGROUND OF THE INVENTION

The following discussion of the background is merely provided to aid the reader in understanding the invention and does not necessarily describe or constitute prior art.

Receptor tyrosine kinases (RTKs) are transmembrane polypeptides that regulate the regeneration, remodeling, development, and differentiation of cells and tissues. See, e.g., Mustonen et al., J. Cell Biology 129, 895-898 (1995); van der Geer et al. Ann Rev. Cell Biol. 10, 251-337 (1994). In addition to activating RTKs, polypeptide ligand growth factors and cytokines are capable of inducing conformation changes in RTK external domains which results in receptor dimerization. Lymboussaki, Academic Dissertation, University of Helsinki, Molecular/Cancer Biology Laboratory and Department of Pathology, Haartman Institute, (1999); Ullrich et al., Cell 61, 203-212 (1990). Cognate RTK receptor-ligand binding, moreover, imparts receptor trans-phosphorylation at specific tyrosine residues and subsequent activation of the kinase catalytic domains, thereby enabling substrate phosphorylation and activation of associated signaling cascades. Id.

Aberrant RTK activity, however, has been associated with a variety of disease conditions relating to, e.g., DNA repair, cell cycle arrest, immunological disorders, angiogenesis, cancer, diabetes, Alzheimer's disease, central nervous system disorders, inflammatory diseases, hyperproliferation of cells, renal and kidney degeneration, bone remodeling, metabolic conditions, and diseases of the vascular system.

The systemic delivery of certain RTK inhibitors, e.g., imatinib, have shown efficacy for specific disease conditions. In vivo assays to this end, including the murine monocrotaline (MCT) model system, have been employed for ascertaining whether putative RTK inhibitors would function as therapeutic agents. Concerning preclinical drug candidate efficacy, however, the MCT model has been criticized inasmuch as such a system fails to substantiate certain human disease phenotypes, e.g., the development of neointimal and/or plexiform lesions that are symptomatically comorbid with such diseases. Hence, the murine MCT model is an imperfect system, which may confound the etiological and/or pathological indications of some human diseases. As such, new or complementary model systems are necessary for accurate and efficient drug development.

In concert with the development and administration of first generation RTK inhibitors, e.g., imatinib, RTKs have evolved inhibitor resistance by acquiring certain mutations. See Shah et al., Science, 305, 395-402 (2004); and LaRosse et al., Cancer Res. 67, 7149-7153 (2002). For example, in diseased patients refractory to certain kinase inhibitors, e.g., imatinib, gefitinib, and erlotinib, it has been shown that the hydrophobic pocket “gatekeeper residue” frequently possesses mutations. See Pao et al., PLos Med. 2(3):e73 (2005). Such mutations have been identified with respect to ABL, i.e., at the T315 residue, and at analogous positions in KIT, PDGFRa, EGFR, and other kinases. Id. A mutant form of FLT3 (D835Y), moreover, has been identified in patients with hematological malignancies. See Yamamoto et al., 97, 2430-2436 (2001).

Accordingly, new RTK inhibitors with superior efficacy—developed in model systems that phenotypically resemble human disease pathology—are required for preventing and treating human conditions associated with RTK activity.

### SUMMARY OF THE INVENTION

The present disclosure is based on the discovery that certain compounds possess RTK inhibitor activity, and that certain RTK inhibitors covalently, i.e., irreversibly, interact with specific targets, which therefore imparts that such activity has clinical value in the prevention and treatment of certain disease states. In one aspect, the present disclosure provides methods of treating pulmonary arterial hypertension (PAH) in a subject or a biological condition associated with PAH in a subject, comprising administering to the subject a therapeutically effective amount of a compound of Structure 1, a tautomer of the compound, a pharmaceutically acceptable salt of the compound, a pharmaceutically acceptable salt of the tautomer, or a mixture thereof, where Structure 1 has the formula:

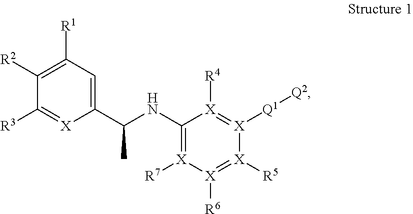

Where X is independently selected from C, N, O, S, and —CN;

and where R¹, R², and R³ may be the same or different and are independently selected from the group consisting of H, C, N, O, S, Cl, Br, F, I, —CN, —NO₂, —OH, —CH₃, —CF₃, —C—N—C— groups, —C—N—C(═O)— groups, —C(═O)R⁸ groups, —N—C(═O)R⁸ groups, —C—N—C(═O)R⁸ groups, substituted and unsubstituted R⁸ groups, substituted and unsubstituted R⁸ groups substituted with one or more of R⁹, R¹⁰, and R¹¹, substituted and unsubstituted amidinyl groups, substituted and unsubstituted guanidinyl groups, substituted and unsubstituted primary, secondary, and tertiary alkyl groups, substituted and unsubstituted aryl groups, substituted and unsubstituted alkenyl groups, substituted and unsubstituted alkynyl groups, substituted and unsubstituted heterocyclyl groups, substituted and unsubstituted aminoalkyl groups, substituted and unsubstituted alkylaminoalkyl groups, substituted and unsubstituted dialkylaminoalkyl groups, substituted and unsubstituted arylaminoalkyl groups, substituted and unsubstituted diarylaminoalkyl groups, substituted and unsubstituted (alkyl)(aryl)aminoalkyl groups, substituted and unsubstituted heterocyclylalkyl groups, substituted and unsubstituted cyano groups, substituted and unsubstituted pyrimidinyl groups, substituted and unsubstituted cyano(aryl) groups, substituted and unsubstituted cyano(heterocyclyl) groups, and substituted and unsubstituted cyano-pyrimidinyl groups;

R⁴, R⁵, R⁶, and R⁷, may be the same or different and are independently selected from the group consisting of H, Cl, Br, F, I, —CN, —NO₂, —OH, —CH₃, —CF₃, —NH₂, —C≡N, —C═N groups, —C—N—C— groups, —C—N—C(═O)— groups, —C—N—C(═O)—C—F, —C—N—C(═O)—C═C, substituted and unsubstituted alkyl groups, substituted and unsubstituted aryl groups, substituted and unsubstituted heterocyclyl groups, alkoxy groups, aryloxy groups, substituted and unsubstituted heterocyclylalkyl groups, substituted and unsubstituted aminoalkyl groups, substituted and unsubstituted alkylaminoalkyl groups, substituted and unsubstituted dialkylaminoalkyl groups, substituted and unsubstituted arylaminoalkyl groups, substituted and unsubstituted diarylaminoalkyl groups, substituted and unsubstituted (alkyl)(aryl)aminoalkyl groups, substituted and unsubstituted alkylamino groups, substituted and unsubstituted arylamino groups, substituted and unsubstituted dialkylamino groups, substituted and unsubstituted diarylamino groups, substituted and unsubstituted (alkyl)(aryl)amino groups, —C(═O)H, —C(═O)-alkyl groups, —C(═O)-aryl groups, —C(═O)O-alkyl groups, —C(═O)O-aryl groups, —C(═O)NH₂, —C(═O)NH(alkyl) groups, —C(═O)NH(aryl) groups, —C(═O)N(alkyl)₂ groups, —C(═O)-aryl groups, —C(═O)NH₂, —C(═O)NH(alkyl) groups, —C(═O)NH(aryl) groups, —C(═O)N(alkyl)₂ groups, —C(═O)N(aryl)₂ groups, —C(═O)N(alkyl)(aryl) groups, —C(═O)O-alkyl groups, —C(═O)O-aryl groups, —C(═O)-heterocyclyl groups, —C(═O)—O-heterocyclyl groups, —C(═O)NH(heterocyclyl) groups, —C(═O)—N(heterocyclyl)₂ groups, —C(═O)—N(alkyl)(heterocyclyl) groups, —C(═O)—N(aryl)(heterocyclyl) groups, substituted and unsubstituted heterocyclylaminoalkyl groups, substituted and unsubstituted hydroxyalkyl groups, substituted and unsubstituted alkoxyalkyl groups, substituted and unsubstituted aryloxyalkyl groups, and substituted and unsubstituted heterocyclyloxyalkyl groups, substituted and unsubstituted diheterocyclylaminoalkyl, substituted and unsubstituted (heterocyclyl)(alkyl)aminoalkyl, substituted and unsubstituted (heterocyclyl) (aryl)aminoalkyl, substituted and unsubstituted alkoxyalkyl groups, substituted and unsubstituted hydroxyalkyl groups, substituted and unsubstituted aryloxyalkyl groups, and substituted and unsubstituted heterocyclyloxyalkyl groups; -(alkyl)(aryl)aminoalkyl groups, —C(═O)-heterocyclyl groups, —C(═O)—O-heterocyclyl groups, —C(═O)NH(heterocyclyl) groups, —C(═O)—N(heterocyclyl)₂ groups, —C(═O)—N(alkyl)(heterocyclyl) groups, —C(═O)—N(aryl)(heterocyclyl) groups, substituted and unsubstituted heterocyclylaminoalkyl groups, substituted and unsubstituted hydroxyalkyl groups, substituted and unsubstituted alkoxyalkyl groups, substituted and unsubstituted aryloxyalkyl groups, and substituted and unsubstituted heterocyclyloxyalkyl groups, —NH(alkyl) groups, —NH(aryl) groups, —N(alkyl)₂ groups, —N(aryl)₂ groups, —N(alkyl)(aryl) groups, —NH(heterocyclyl) groups, —N(heterocyclyl)(alkyl) groups, —N(heterocyclyl)(aryl) groups, —N(heterocyclyl)₂ groups, substituted and unsubstituted alkyl groups, substituted and unsubstituted aryl groups, —OH, substituted and unsubstituted alkoxy groups, substituted and unsubstituted aryloxy groups, substituted and unsubstituted heterocyclyl groups, —NHOH, —N(alkyl)OH groups, —N(aryl)OH groups, —N(alkyl)O-alkyl groups, —N(aryl)O-alkyl groups, —N(alkyl)O-aryl groups, and —N(aryl)O-aryl groups;

R⁸ is selected from the group consisting of R¹, R², R³, R⁴, R⁵, R⁶, R⁷, H, absent, —C═C, substituted and unsubstituted heterocyclyl groups, substituted and unsubstituted aryl groups, substituted and unsubstituted heterocyclyl(R⁹) groups, substituted and unsubstituted heterocyclyl(R¹⁰) groups, substituted and unsubstituted heterocyclyl(R¹¹) groups, substituted and unsubstituted heterocyclyl(R⁹)(R¹⁰) groups, substituted and unsubstituted heterocyclyl(R⁹)(R¹¹) groups, substituted and unsubstituted heterocyclyl(R¹⁰)(R¹¹) groups, substituted and unsubstituted heterocyclyl(R⁹)(R¹⁰)(R¹¹) groups, substituted and unsubstituted —C(═O)-heterocyclyl(R⁹) groups, substituted and unsubstituted —C(═O)-heterocyclyl(R¹⁰) groups, substituted and unsubstituted —C(═O)-heterocyclyl(R¹¹) groups, substituted and unsubstituted —C(═O)-heterocyclyl(R⁹)(R¹⁰) groups, substituted and unsubstituted —C(═O)-heterocyclyl(R⁹)(R¹¹) groups, substituted and unsubstituted —C(═O)-heterocyclyl(R¹⁰)(R¹¹) groups, substituted and unsubstituted —C(═O)-heterocyclyl(R⁹)(R¹⁰)(R¹¹) groups, substituted and unsubstituted aryl(R⁹) groups, substituted and unsubstituted aryl(R¹⁰) groups, substituted and unsubstituted aryl (R¹¹) groups, substituted and unsubstituted aryl (R⁹)(R¹⁰) groups, substituted and unsubstituted aryl (R⁹)(R¹¹) groups, substituted and unsubstituted aryl(R¹⁰)(R¹¹) groups, substituted and unsubstituted aryl (R⁹)(R¹⁰)(R¹¹) groups, substituted and unsubstituted —C(═O)-aryl(R⁹) groups, substituted and unsubstituted —C(═O)-aryl(R¹⁰) groups, substituted and unsubstituted —C(═O)-aryl(R¹¹) groups, substituted and unsubstituted —C(═O)-aryl(R⁹)(R¹⁰) groups, substituted and unsubstituted —C(═O)-aryl(R⁹)(R¹¹) groups, substituted and unsubstituted —C(═O)-aryl(R¹⁰)(R¹¹) groups, and substituted/unsubstituted —C(═O)-aryl(R⁹)(R¹⁰)(R¹¹) groups;

R⁹, R¹⁰, and R¹¹ may be the same or different and are independently selected from the group consisting of absent, H, Cl, Br, F, I, —CN, —NO₂, —OH, —CH₃, —CF₃, —NH₂, —C(═O)—, —C—N—R¹², —C≡N, —C—N—C groups, —C—N—C(═O)— groups, —C—N—C(═O)—C—F, —C—N—C(═O)—C═C, —C═N groups, substituted and unsubstituted alkyl groups, substituted and unsubstituted aryl groups, substituted and unsubstituted heterocyclyl groups, alkoxy groups, aryloxy groups, substituted and unsubstituted heterocyclylalkyl groups, substituted and unsubstituted aminoalkyl groups, substituted and unsubstituted alkylaminoalkyl groups, substituted and unsubstituted dialkylaminoalkyl groups, substituted and unsubstituted arylaminoalkyl groups, substituted and unsubstituted diarylaminoalkyl groups, substituted and unsubstituted (alkyl)(aryl)aminoalkyl groups, substituted and unsubstituted alkylamino groups, substituted and unsubstituted arylamino groups, and substituted and unsubstituted dialkylamino groups, substituted and unsubstituted aminoalkyl groups, substituted and unsubstituted (alkyl)(aryl)aminoalkyl groups, substituted and unsubstituted alkylamino groups, substituted and unsubstituted arylamino groups, substituted and unsubstituted dialkylamino groups, substituted and unsubstituted diarylamino groups, substituted and unsubstituted (alkyl)(aryl)amino groups, —C(═O)H, —C(═O)-alkyl groups, —C(═O)-aryl groups, —C(═O)O-alkyl groups, —C(═O)O-aryl groups, —C(═O)NH₂, —C(═O)NH(alkyl) groups, —C(═O)NH(aryl) groups, —C(═O)N(alkyl)₂ groups, —C(═O)-aryl groups, —C(═O)NH₂, —C(═O)NH(alkyl) groups, —C(═O)NH(aryl) groups, —C(═O)N(alkyl)₂ groups, —C(═O)N(aryl)₂ groups, —C(═O)N(alkyl)(aryl) groups, —C(═O)O-alkyl groups, —C(═O)O-aryl groups, —C(═O)-heterocyclyl groups, —C(═O)—O-heterocyclyl groups, —C(═O)NH(heterocyclyl) groups, —C(═O)—N(heterocyclyl)₂ groups, —C(═O)—N(alkyl)(heterocyclyl) groups, —C(═O)—N(aryl)(heterocyclyl) groups, substituted and unsubstituted heterocyclylaminoalkyl groups, substituted and unsubstituted cyano groups, substituted and unsubstituted pyrimidinyl groups, substituted and unsubstituted cyano(aryl) groups, substituted and unsubstituted cyano(heterocyclyl) groups, and substituted and unsubstituted cyano-pyrimidinyl groups;

R¹² is selected from the group consisting of absent, H, Cl, Br, F, I, —CN, —NO₂, —OH, —CH₃, —CF₃, —NH₂, —C(═O)—, —C—N—R¹², —C≡N, —C—N—C groups, —C—N—C(═O)— groups, —C—N—C(═O)—C—F, —C—N—C(═O)—C═C, —C═N groups, —C(═O)— groups, —C(═O)—C— groups, —C(═O)—C═C, —S(═O)₂— groups, —S(═O)₂—C— groups, —S(═O)₂—C═C— groups, —S(═O)₂—C═C—CH₃, alkoxy groups, aryloxy groups, substituted and unsubstituted amidinyl groups, substituted and unsubstituted guanidinyl groups, substituted and unsubstituted primary, secondary, and tertiary alkyl groups, substituted and unsubstituted aryl groups, substituted and unsubstituted alkenyl groups, substituted and unsubstituted alkynyl groups, substituted and unsubstituted heterocyclyl groups, substituted and unsubstituted aminoalkyl groups, substituted and unsubstituted alkylaminoalkyl groups, substituted and unsubstituted dialkylaminoalkyl groups, substituted and unsubstituted arylaminoalkyl groups, substituted and unsubstituted diarylaminoalkyl groups, substituted and unsubstituted (alkyl)(aryl)aminoalkyl groups, substituted and unsubstituted heterocyclylalkyl groups, substituted and unsubstituted cyano groups, substituted and unsubstituted pyrimidinyl groups, substituted and unsubstituted cyano(aryl) groups, substituted and unsubstituted cyano(heterocyclyl) groups, and substituted and unsubstituted cyano-pyrimidinyl groups;

Q¹ is selected from the group consisting of a direct bond, H, C, Cl, Br, F, I, —CN, —NO₂, —CH₃, —CF₃, —NH₂, —C(═O)—, —C—N—R¹², —C≡N, —C—N—C groups, —C—N—C(═O)— groups, —C—N—C(═O)—C—F, —C—N—C(═O)—C═C, —C═N groups, —C(═O)— groups, —C(═O)—C— groups, —C(═O)—C═C, —CF₃, —C≡N, —C—N—C— groups, —C—N—C(═O)— groups, —C—N—C(═O)—C—F, —C—N—C(═O)—C═C, —OH, alkoxy groups, aryloxy groups, substituted and unsubstituted alkyl groups, substituted and unsubstituted aryl groups, substituted and unsubstituted heterocyclyl groups, —OH, alkoxy groups, aryloxy groups, methoxymethyl benzyl groups, substituted and unsubstituted aralkyl groups, —NH₂, substituted and unsubstituted heterocyclylalkyl groups, substituted and unsubstituted aminoalkyl groups, substituted and unsubstituted alkylaminoalkyl groups, substituted and unsubstituted dialkylaminoalkyl groups, substituted and unsubstituted arylaminoalkyl groups, substituted and unsubstituted diarylaminoalkyl groups, substituted and unsubstituted (alkyl)(aryl)aminoalkyl groups, substituted and unsubstituted alkylamino groups, substituted and unsubstituted arylamino groups, and substituted and unsubstituted dialkylamino groups, substituted and unsubstituted cyano groups, substituted and unsubstituted pyrimidinyl groups, substituted and unsubstituted cyano(aryl) groups, substituted and unsubstituted cyano(heterocyclyl) groups, and substituted and unsubstituted cyano-pyrimidinyl groups;

Q² is selected from the groups consisting of absent, H, Q¹, Q¹(Q³), —OH, alkoxy groups, aryloxy groups; and

Q³ is selected from the group consisting of absent, a direct bond, H, C, Cl, Br, F, I, —CN, —NO₂, —CH₃, —CF₃, —NH₂, —C(═O)—, —C—N—R¹², —C≡N, —C—N—C groups, —C—N—C(═O)— groups, —C—N—C(═O)—C—F, —C—N—C(═O)—C═C, —C═N groups, —C(═O)— groups, —C(═O)—C— groups, —C(═O)—C═C, —CF₃, —C≡N, —C—N—C— groups, —C—N—C(═O)— groups, —C—N—C(═O)—C—F, —C—N—C(═O)—C═C, —OH, alkoxy groups, alkoxy groups, aryloxy groups, substituted and unsubstituted alkyl groups, substituted and unsubstituted aryl groups, substituted and unsubstituted heterocyclyl groups, —OH, alkoxy groups, and aryloxy groups. The contents of the foregoing paragraph (i.e., [0011]) are hereinafter referred to as “XRQ”.

In some embodiments, the structure of R⁸ has the following formula:

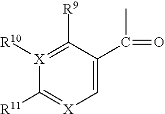

Where X is independently selected from the group consisting of C, N, O, S, and —CN;

R⁹, R¹⁰, and R¹¹ may be the same or different and are independently selected from the group consisting of H, C, N, O, S, Cl, Br, F, I, —CN, —NO₂, —OH, —CH₃, —CF₃, —NH₂, —C(═O)—, —C—N—R¹², —C≡N, —C—N—C(═O)—C—F, —C—N—C(═O)—C═C, substituted and unsubstituted alkyl groups, substituted and unsubstituted aryl groups, substituted and unsubstituted heterocyclyl groups, —OH, alkoxy groups, aryloxy groups, substituted and unsubstituted heterocyclylalkyl groups, substituted and unsubstituted aminoalkyl groups, substituted and unsubstituted alkylaminoalkyl groups, substituted and unsubstituted dialkylaminoalkyl groups, substituted and unsubstituted arylaminoalkyl groups, substituted and unsubstituted diarylaminoalkyl groups, substituted and unsubstituted (alkyl)(aryl)aminoalkyl groups, substituted and unsubstituted alkylamino groups, substituted and unsubstituted arylamino groups, and substituted and unsubstituted dialkylamino groups, substituted and unsubstituted cyano groups, substituted and unsubstituted pyrimidinyl groups, substituted and unsubstituted cyano(aryl) groups, substituted and unsubstituted cyano(heterocyclyl) groups, and substituted and unsubstituted cyano-pyrimidinyl groups; and

R¹² is selected from the group consisting of —C(═O)— groups, —C(═O)—C— groups, —C(═O)—C═C, —S(═O)₂— groups, —S(═O)₂—C— groups, —S(═O)₂—C═C— groups, —S(═O)₂—C═C—CH₃, —OH, alkoxy groups, aryloxy groups, substituted and unsubstituted amidinyl groups, substituted and unsubstituted guanidinyl groups, substituted and unsubstituted primary, secondary, and tertiary alkyl groups, substituted and unsubstituted aryl groups, substituted and unsubstituted alkenyl groups, substituted and unsubstituted alkynyl groups, substituted and unsubstituted heterocyclyl groups, substituted and unsubstituted aminoalkyl groups, substituted and unsubstituted alkylaminoalkyl groups, substituted and unsubstituted dialkylaminoalkyl groups, substituted and unsubstituted arylaminoalkyl groups, substituted and unsubstituted diarylaminoalkyl groups, substituted and unsubstituted (alkyl)(aryl)aminoalkyl groups, substituted and unsubstituted heterocyclylalkyl groups, substituted and unsubstituted cyano groups, substituted and unsubstituted pyrimidinyl groups, substituted and unsubstituted cyano(aryl) groups, substituted and unsubstituted cyano(heterocyclyl) groups, and substituted and unsubstituted cyano-pyrimidinyl groups. The contents of the foregoing paragraph (i.e., [0013]) are hereinafter referred to as “XRQ2”.

In some embodiments, the structure of Q¹ and/or Q² is selected from the group consisting of —C—N—C(═O)—C═C, —C—N—C(═O)—C—F,

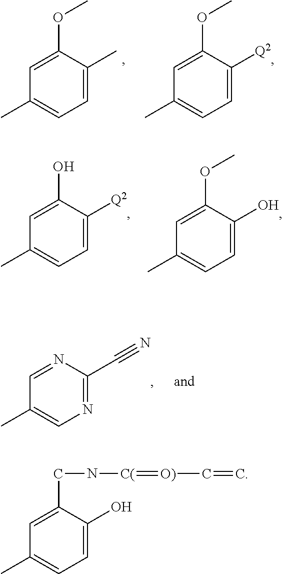

In some embodiments, the compound of Structure 1 is a compound of Structure 2:

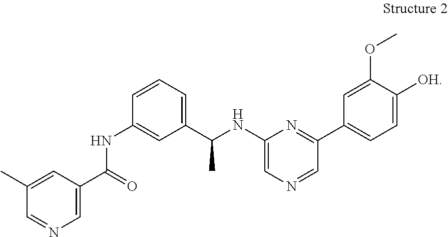 [SMILES: COc1cc(-c2cncc(N[C@@H](C)c3cccc(NC(=O)c4cncc(C)c4)c3)n2)ccc1O]

In some embodiments, the compound of Structure 1 or Structure 2 is administered orally, intravenously, subcutaneously, transdermally, intraperitoneally, or by inhalation. In suitable embodiments, the compound of Structure 1 is administered by inhalation. In some embodiments, PAH is characterized by neointimal lesions or plexiform lesions, or both. In some embodiments, PAH is selected from the group consisting of primary PAH, idiopathic PAH, heritable PAH, refractory PAH, BMPR2, ALK1, endoglin associated with hereditary hemorrhagic telangiectasia, endoglin not associated with hereditary hemorrhagic telangiectasia, drug-induced PAH, and toxin-induced PAH, PAH associated with systemic sclerosis, mixed connective tissue disease, HIV infection, hepatitis, and portal hypertension.

PAH, in some embodiments, is secondary to pulmonary hypertension, congenital heart disease, hypoxia, chronic hemolytic anemia, newborn persistent pulmonary hypertension, pulmonary veno-occlusive disease (PVOD), pulmonary capillary hemangiomatosis (PCH), left heart disease pulmonary hypertension, systolic dysfunction, diastolic dysfunction, valvular disease, lung disease, interstitial lung disease, pulmonary fibrosis, schistosomiasis, chronic obstructive pulmonary disease (COPD), sleep-disordered breathing, alveolar hypoventilation disorders, chronic exposure to high altitude, developmental abnormalities, chronic thromboembolic pulmonary hypertension (CTEPH), pulmonary hypertension with unclear multifactorial mechanisms, hematologic disorders, myeloproliferative disorders, splenectomy, systemic disorders, sarcoidosis, pulmonary Langerhans cell histiocytosis, lymphangioleimoyomatosis, neurofibromatosis, vasculitis, metabolic disorders, glycogen storage disease, Gaucher disease, thyroid disorders, tumoral obstruction, fibrosing mediastinitis, and chronic renal failure on dialysis.

In some embodiments, the biological condition associated with PAH is selected from the group consisting of abnormal: right ventricular systolic pressure (RVSP); pulmonary pressure; cardiac output; right ventricular (RV) hypertrophy; and pulmonary arterial (PA) hypertrophy. In suitable embodiments, the salt is a sulfate, phosphate, mesylate, bismesylate, tosylate, lactate, tartrate, malate, bis-acetate, citrate, or bishydrochloride salt. In some embodiments, the compound of Structure 1, the tautomer of the compound, the pharmaceutically acceptable salt of the compound, the pharmaceutically acceptable salt of the tautomer, or the mixture thereof, is administered in combination with a second drug. The second drug may be selected from the group consisting of prostanoids, endothelin antagonists, PDE5 inhibitors, cytoplasmic kinase inhibitors, and receptor kinase inhibitors.

In suitable embodiments, the therapeutically effective amount of a compound of Structure 1, the tautomer of the compound, the pharmaceutically acceptable salt of the compound, the pharmaceutically acceptable salt of the tautomer, or the mixture thereof, is not associated with adverse side effects. Such adverse side effects comprise one or more of decreased lung function, increased or decreased systemic blood pressure, immunocompromised, bone marrow suppression, anemia, hypoxia, in the subject compared to before the administering.

In some embodiments, the compound of Structure 1, the tautomer of the compound, the pharmaceutically acceptable salt of the compound, the pharmaceutically acceptable salt of the tautomer, or the mixture thereof, covalently interacts with a receptor tyrosine kinase (RTK). The RTK, in some embodiments, is PDGFR or cKit or both. In some embodiments, the PDGFR is selected from the group consisting of PDGFR-α, PDGFR-β, PDGFR-αα, PDGFR-ββ, and PDGFR-αβ. Moreover, the compound of Structure 1, in suitable embodiments, covalently interacts with one or more of the PDGFR-α, PDGFR-β, PDGFR-αα, PDGFR-ββ, and/or the PDGFR-αβ amino acids selected from the group consisting of LYS627, VAL607, GLU644, MET648, HIS816, LEU809, ASP836, CYS814, ILE834, CYS835, PHE937, LYS634, VAL614, GLU651, MET655, HIS824, LEU817, ASP844, CYS822, ILE842, VAL658, ILE647, HIS816, ARG836, LYS634, GLU651, ALA632, HIS824, MET655, ARG825, CYS843, THR874, ARG817, VAL815, LEU651, LEU809, ILE657, THR681, ILE654, ARG825, ASP826, LEU658, LEU825, PHE837, LEU658, HIS824, CYS814, ILE654, ASP844, ILE842, and CYS843.

In some embodiments, the compound of Structure 1, the tautomer of the compound, the pharmaceutically acceptable salt of the compound, the pharmaceutically acceptable salt of the tautomer, or the mixture thereof is administered in a total daily dosage from about 0.001 mg/kg to about 1 mg/kg by inhalation or oral. In some embodiments, the compound of Structure I, the tautomer of the compound, the pharmaceutically acceptable salt of the compound (or the tautomer), or the mixture thereof, is administered from one to five times daily.

Likewise, in some embodiments, the compound of Structure I, the tautomer of the compound, the pharmaceutically acceptable salt of the compound, the pharmaceutically acceptable salt of the tautomer, or the mixture thereof, is administered in unit dosage form, where the unit dose comprises from about 0.001 mg/kg to about 1 mg/kg of the compound, tautomer, and/or salts based on the subject's body weight, or from about 0.01 mg/kg to about 100 mg of the compound, tautomer, and/or salts by inhalation or oral delivery.

In some embodiments, the unit dose is sufficient to provide one or more of: (a) a Cₘₐₓ of about 1 to 5000 ng/mL of the compound In a subject's plasma or a Cₘₐₓ of about 1 to 5000 ng/mL of the compound In the subject's blood when it is administered to the subject; and (b) about 1 to 5000 ng/mL of the compound in a subject's plasma 24 hours after administration or about 1 to 5000 ng/mL of the compound in the subject's blood 24 hours after administration to the subject. In some embodiments, the subject is a human subject.

In suitable embodiments, the compound of Structure 1 is a compound of Structure 3, 4, 5, 6, 7, 8, 9, 10, 11, or 12, as shown below in Chart A, where R is H, F, CH₃, or CF₃.

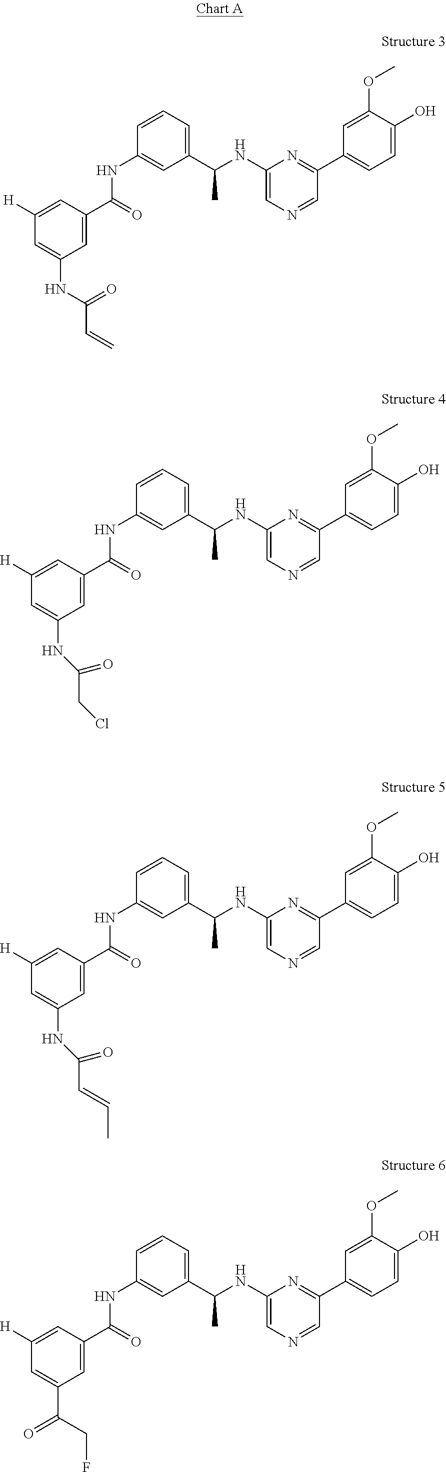 [SMILES: C=CC(=O)Nc1cccc(C(=O)Nc2cccc([C@H](C)Nc3cncc(-c4ccc(O)c(OC)c4)n3)c2)c1 ; COc1cc(-c2cncc(N[C@@H](C)c3cccc(NC(=O)c4cccc(NC(=O)CCl)c4)c3)n2)ccc1O ; C/C=C/C(=O)Nc1cccc(C(=O)Nc2cccc([C@H](C)Nc3cncc(-c4ccc(O)c(OC)c4)n3)c2)c1 ; COc1cc(-c2cncc(N[C@@H](C)c3cccc(NC(=O)c4cccc(C(=O)CF)c4)c3)n2)ccc1O]

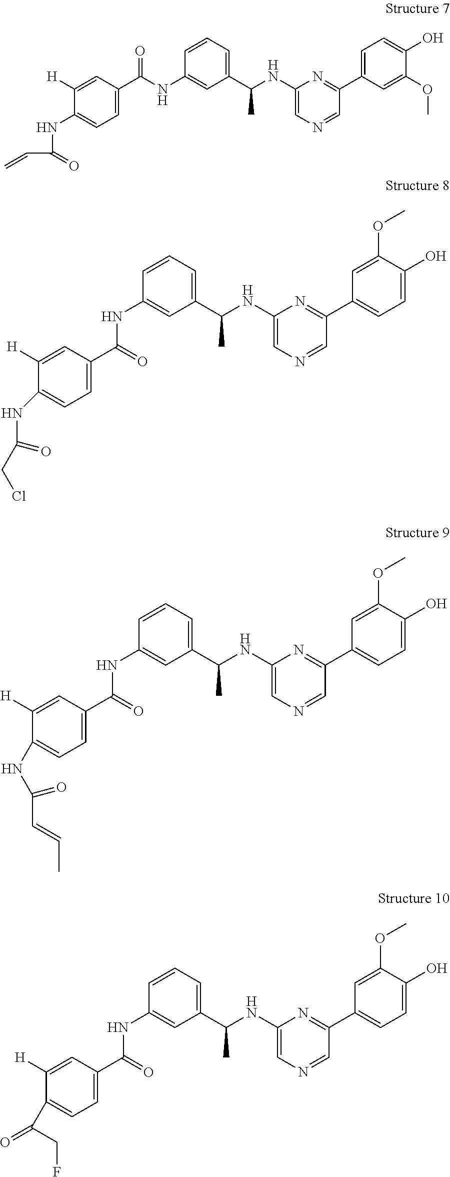 [SMILES: C=CC(=O)Nc1ccc(C(=O)Nc2cccc([C@H](C)Nc3cncc(-c4ccc(O)c(OC)c4)n3)c2)cc1 ; COc1cc(-c2cncc(N[C@@H](C)c3cccc(NC(=O)c4ccc(NC(=O)CCl)cc4)c3)n2)ccc1O ; C/C=C/C(=O)Nc1ccc(C(=O)Nc2cccc([C@H](C)Nc3cncc(-c4ccc(O)c(OC)c4)n3)c2)cc1 ; COc1cc(-c2cncc(N[C@@H](C)c3cccc(NC(=O)c4ccc(C(=O)CF)cc4)c3)n2)ccc1O]

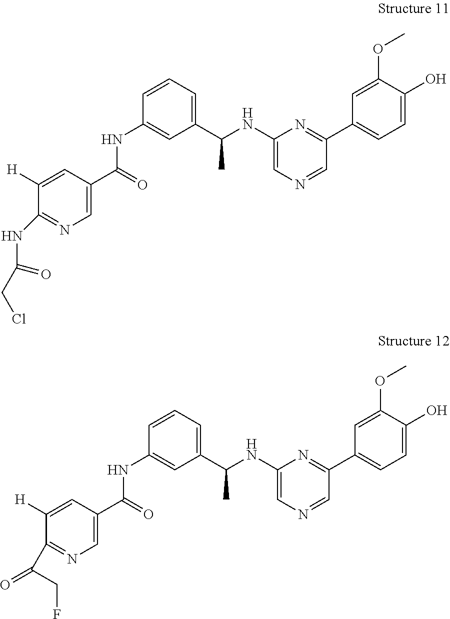 [SMILES: COc1cc(-c2cncc(N[C@@H](C)c3cccc(NC(=O)c4ccc(NC(=O)CCl)nc4)c3)n2)ccc1O ; COc1cc(-c2cncc(N[C@@H](C)c3cccc(NC(=O)c4ccc(C(=O)CF)nc4)c3)n2)ccc1O]

The compound of Structure 1, moreover, is a compound of Structure 13, 14, 15, 16, 17, 18, 19, 20, 21, 22, 23, 24, 25, 26, 27, 28, 29, 30, 31, 32, 33, or 34, in some embodiments, as shown below in Chart B.

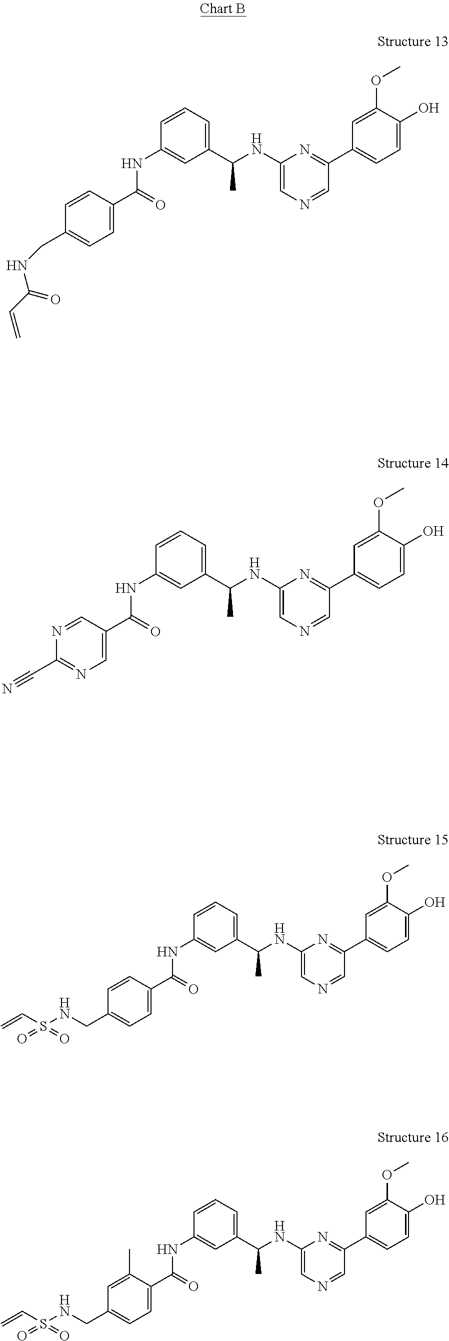 [SMILES: C=CC(=O)NCc1ccc(C(=O)Nc2cccc([C@H](C)Nc3cncc(-c4ccc(O)c(OC)c4)n3)c2)cc1 ; COc1cc(-c2cncc(N[C@@H](C)c3cccc(NC(=O)c4cnc(C#N)nc4)c3)n2)ccc1O ; C=CS(=O)(=O)NCc1ccc(C(=O)Nc2cccc([C@H](C)Nc3cncc(-c4ccc(O)c(OC)c4)n3)c2)cc1 ; C=CS(=O)(=O)NCc1ccc(C(=O)Nc2cccc([C@H](C)Nc3cncc(-c4ccc(O)c(OC)c4)n3)c2)c(C)c1]

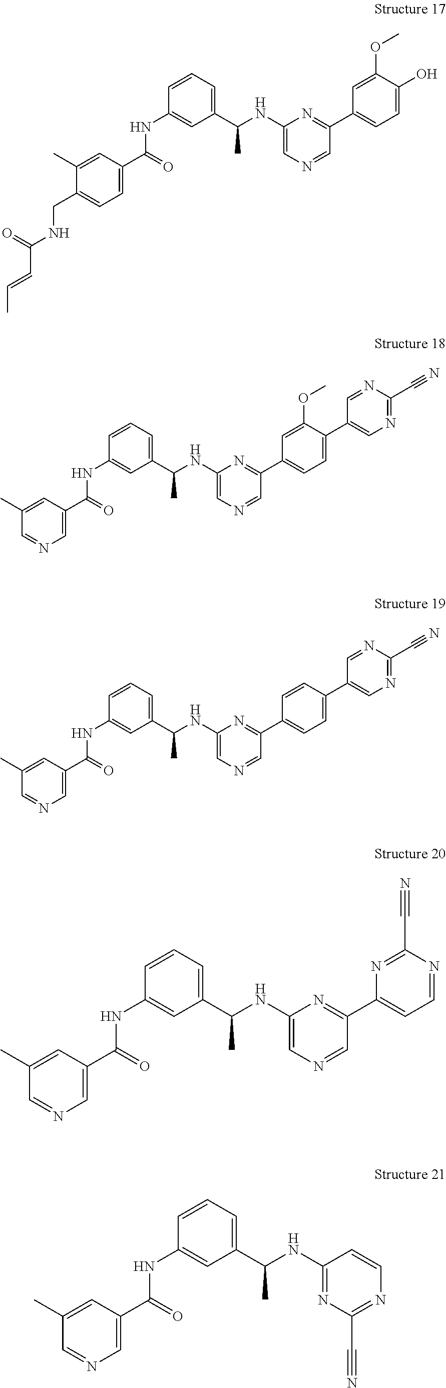 [SMILES: C/C=C/C(=O)NCc1ccc(C(=O)Nc2cccc([C@H](C)Nc3cncc(-c4ccc(O)c(OC)c4)n3)c2)cc1C ; COc1cc(-c2cncc(N[C@@H](C)c3cccc(NC(=O)c4cncc(C)c4)c3)n2)ccc1-c1cnc(C#N)nc1 ; Cc1cncc(C(=O)Nc2cccc([C@H](C)Nc3cncc(-c4ccc(-c5cnc(C#N)nc5)cc4)n3)c2)c1 ; Cc1cncc(C(=O)Nc2cccc([C@H](C)Nc3cncc(-c4ccnc(C#N)n4)n3)c2)c1 ; Cc1cncc(C(=O)Nc2cccc([C@H](C)Nc3ccnc(C#N)n3)c2)c1]

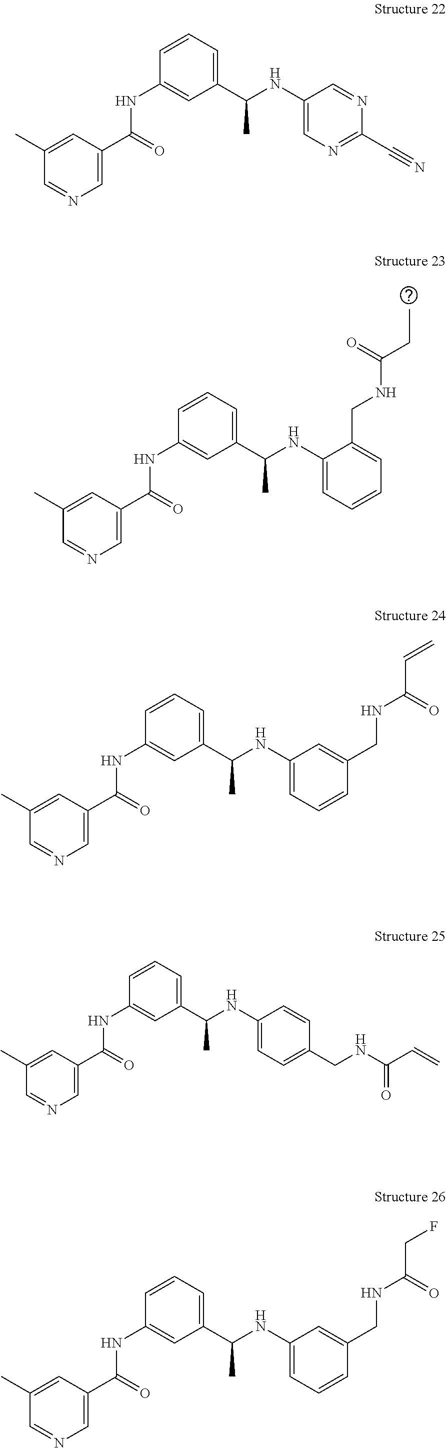

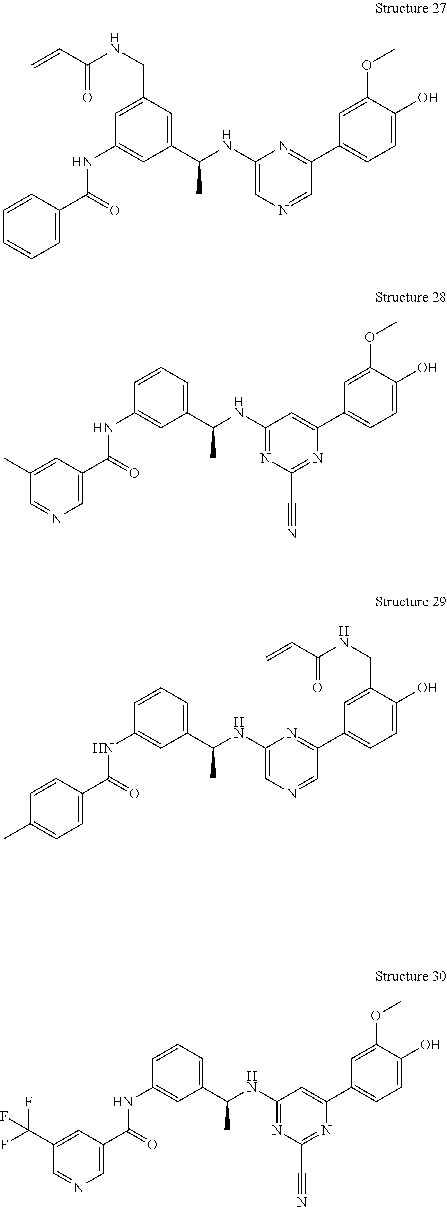 [SMILES: C=CC(=O)NCc1cc(NC(=O)c2ccccc2)cc([C@H](C)Nc2cncc(-c3ccc(O)c(OC)c3)n2)c1 ; COc1cc(-c2cc(N[C@@H](C)c3cccc(NC(=O)c4cncc(C)c4)c3)nc(C#N)n2)ccc1O ; C=CC(=O)NCc1cc(-c2cncc(N[C@@H](C)c3cccc(NC(=O)c4ccc(C)cc4)c3)n2)ccc1O ; COc1cc(-c2cc(N[C@@H](C)c3cccc(NC(=O)c4cncc(C(F)(F)F)c4)c3)nc(C#N)n2)ccc1O]

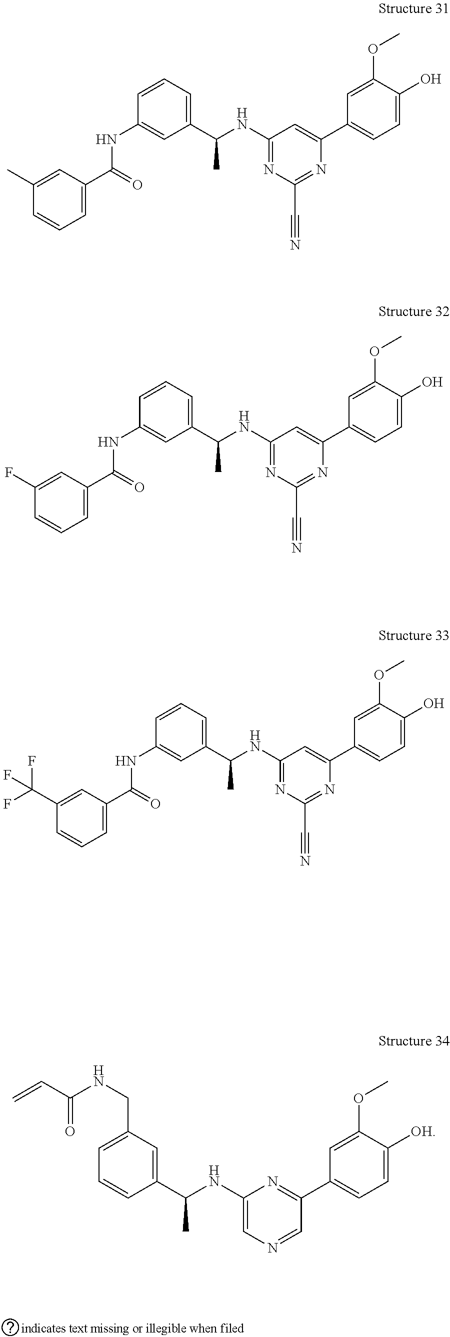 [SMILES: COc1cc(-c2cc(N[C@@H](C)c3cccc(NC(=O)c4cccc(C)c4)c3)nc(C#N)n2)ccc1O ; COc1cc(-c2cc(N[C@@H](C)c3cccc(NC(=O)c4cccc(F)c4)c3)nc(C#N)n2)ccc1O ; COc1cc(-c2cc(N[C@@H](C)c3cccc(NC(=O)c4cccc(C(F)(F)F)c4)c3)nc(C#N)n2)ccc1O ; C=CC(=O)NCc1cccc([C@H](C)Nc2cncc(-c3ccc(O)c(OC)c3)n2)c1]

In one aspect, the present invention provides methods of preventing or reducing elevated pulmonary pressure in a subject, comprising administering to the subject a therapeutically effective amount of a compound of Structure 1, a tautomer of the compound, a pharmaceutically acceptable salt of the compound, a pharmaceutically acceptable salt of the tautomer, or a mixture thereof, where the elevated pulmonary pressure in the subject is elevated compared to a healthy subject in a control population, and further where the reduced pulmonary pressure is reduced compared to the pulmonary pressure in the subject prior to the administering, where Structure 1 has the following formula:

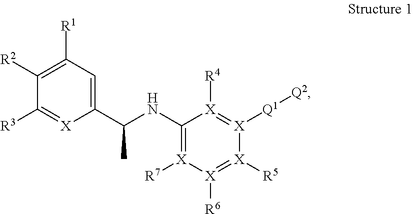

where X, R¹, R², R³, R⁴, R⁵, R⁶, R⁷ (and R⁸, R⁹, R¹⁰, R¹¹, and R¹², as contained therein), Q¹ and Q² (and Q³ as contained therein) are selected from “XRQ”, as noted above in the Summary.

In some embodiments, the structure of R⁸ has the following formula:

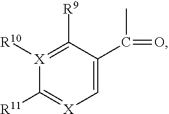

where X, R⁹, R¹⁰, R¹¹ and R¹² are selected from “XRQ2”, as noted in the Summary above.

The structure of Q¹ and/or Q², in some embodiments, is selected from the group consisting of —C—N—C(═O)—C═C, —C—N—C(═O)—C—F,

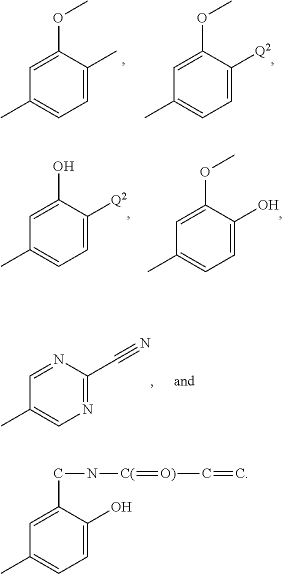

The compound of Structure 1, in some embodiments, is a compound of Structure 2:

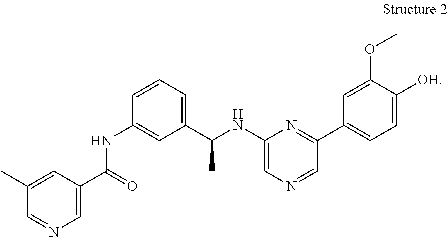 [SMILES: COc1cc(-c2cncc(N[C@@H](C)c3cccc(NC(=O)c4cncc(C)c4)c3)n2)ccc1O]

In some embodiments, the reduction in pulmonary pressure is associated with an increase in one or more of RV function, PA systolic pressure, and cardiac output in the subject compared to the subject prior to the administering. In some embodiments, the reduction in pulmonary pressure is associated with a decrease in one or more of RV hypertrophy, PA hypertrophy, RVSP, sustained PA pressure, and the risk of stroke in the subject compared to the subject prior to the administering. The decrease is at least a 40% decrease, in some embodiments. In some embodiments, the reduction in pulmonary pressure is not associated with decreased lung function and increased systemic blood pressure in the subject compared to the subject prior to the administering. In some embodiments, the reduction in pulmonary pressure is a decrease in pulmonary arterial pressure in the subject compared to the subject prior to the administering.

In one aspect, the present invention provides methods of treating pulmonary arterial hypertension (PAH) in a subject by modulating kinase phosphorylation-state ratios (PSR) or kinase receptor activity in the subject, comprising: administering to the subject a compound of Structure 1, a tautomer of the compound, a pharmaceutically acceptable salt of the compound, a pharmaceutically acceptable salt of the tautomer, or a mixture thereof, wherein the kinase or the receptor kinase is selected from the group consisting of c-Kit, PDGF, PDGFR, STAT3, ERK1, and ERK2, or any other RTK, and where the compound of Structure 1 has the following formula:

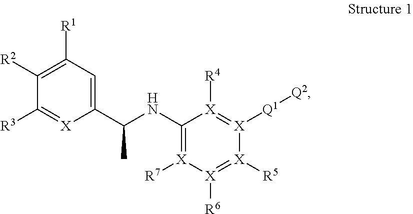

where X, R¹, R², R³, R⁴, R⁵, R⁶, R⁷ (and R⁸, R⁹, R¹⁰, R¹¹, and R¹², as contained therein), Q¹ and Q² (and Q³ as contained therein) are selected from “XRQ”, as noted in the Summary above.

In some embodiments, the structure of R⁸ has the following formula:

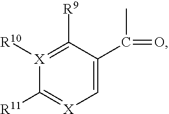

where X, R⁹, R¹⁰, R¹¹ and R¹² are selected from “XRQ2”, as noted above in the Summary. In some embodiments, the structure of Q¹ and/or Q² is selected from the group consisting of —C—N—C(═O)—C═C, —C—N—C(═O)—C—F, or as follows below:

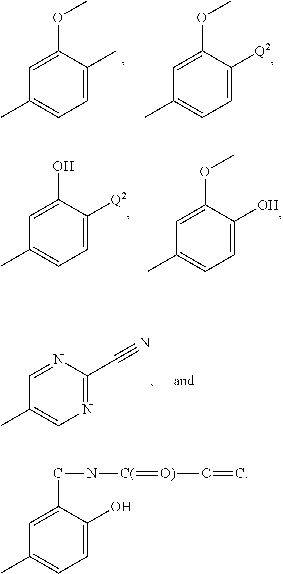

In some embodiments, the compound of Structure 1 is a compound of Structure 2:

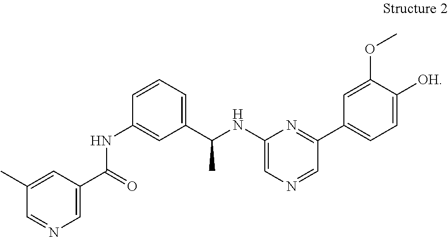 [SMILES: COc1cc(-c2cncc(N[C@@H](C)c3cccc(NC(=O)c4cncc(C)c4)c3)n2)ccc1O]

In some embodiments, the modulation of the kinase receptor activity is an inhibition of the kinase receptor activity, and wherein the kinase receptor is PDGFR or c-Kit. In some embodiments, the PDGFR is selected from the group consisting of PDGFR-α, PDGFR-β, PDGFR-αα, PDGFR-ββ, and PDGFR-αβ. The modulation of the PSR is a modulation of one or more of STAT3, ERK1, ERK2, PDGF, and PDGFR, in suitable embodiments.

In some embodiments, the modulation of PSR is a decrease of phosphorylated STAT3 to total STAT3 in the subject compared to the PSR in the subject prior to the administering. In some embodiments, the modulation of PSR is a decrease of diphosphorylated ERK1 to total ERK1 in the subject compared to the PSR in the subject prior to the administering. In some embodiments, the modulation of PSR is a decrease of diphosphorylated ERK2 to total ERK2 in the subject compared to the PSR in the subject prior to the administering. In some embodiments, the modulation of PSR is a decrease of monophosphorylated ERK1 to total ERK1 in the subject compared to the PSR in the subject prior to the administering. In some embodiments, the modulation of PSR is a decrease of phosphorylated PDGFR to total PDGFR in the subject compared to the PSR in the subject prior to the administering.

In one aspect, the present disclosure provides a compound of Structure 1, a tautomer of the compound, a pharmaceutically acceptable salt of the compound, a pharmaceutically acceptable salt of the tautomer, or a mixture thereof, for treating one or more diseases associated with hyperproliferation, neoplasia, hypoplasia, hyperplasia, dysplasia, metaplasia, prosoplasia, desmoplasia, angiogenesis, inflammation, immunological state, metabolism, pulmonary function, and cardiovascular function, wherein Structure 1 has the following formula:

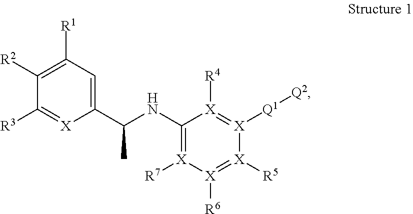

where X, R¹, R², R³, R⁴, R⁵, R⁶, R⁷ (and R⁸, R⁹, R¹⁰, R¹¹, and R¹², as contained therein), Q¹ and Q² (and Q³ as contained therein) are selected from “XRQ”, as noted above in the Summary.

In some embodiments, the structure of R⁸ has the following formula:

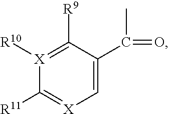

where X, R⁹, R¹⁰, R¹¹ and R¹² are selected from “XRQ2”, as noted above in the Summary.

In some embodiments, the structure of Q¹ and/or Q² is selected from the group consisting of —C—N—C(═O)—C═C, —C—N—C(═O)—C—F,

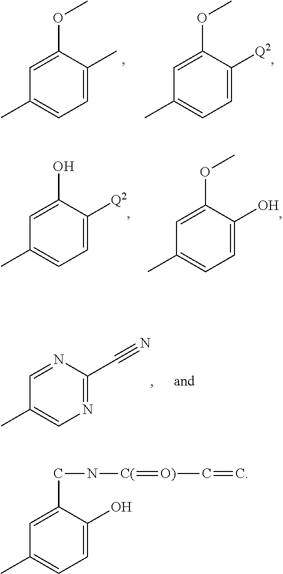

In suitable embodiments, the compound of Structure 1 is a compound of Structure 2:

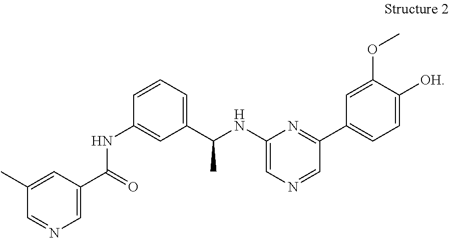 [SMILES: COc1cc(-c2cncc(N[C@@H](C)c3cccc(NC(=O)c4cncc(C)c4)c3)n2)ccc1O]

In some embodiments, the compound of Structure 1 or Structure 2 is administered orally, intravenously, subcutaneously, transdermally, intraperitoneally, or by inhalation. The compound of Structure 1 is administered by inhalation in suitable embodiments.

In some embodiments, the disease is selected from the group consisting of cancer, metastatic cancer, HIV, hepatitis, PAH, primary PAH, idiopathic PAH, heritable PAH, refractory PAH, BMPR2, ALK1, endoglin associated with hereditary hemorrhagic telangiectasia, endoglin not associated with hereditary hemorrhagic telangiectasia, drug-induced PAH, and toxin-induced PAH, PAH associated with systemic sclerosis, and mixed connective tissue disease, pulmonary hypertension, congenital heart disease, hypoxia, chronic hemolytic anemia, newborn persistent pulmonary hypertension, pulmonary veno-occlusive disease (PVOD), pulmonary capillary hemangiomatosis (PCH), left heart disease pulmonary hypertension, systolic dysfunction, diastolic dysfunction, valvular disease, lung disease, interstitial lung disease, pulmonary fibrosis, schistosomiasis, chronic obstructive pulmonary disease (COPD), sleep-disordered breathing, alveolar hypoventilation disorders, chronic exposure to high altitude, developmental abnormalities, chronic thromboembolic pulmonary hypertension (CTEPH), pulmonary hypertension with unclear multifactorial mechanisms, hematologic disorders, myeloproliferative disorders, splenectomy, systemic disorders, sarcoidosis, pulmonary Langerhans cell histiocytosis, lymphangioleimoyomatosis, neurofibromatosis, vasculitis, metabolic disorders, glycogen storage disease, Gaucher disease, thyroid disorders, tumoral obstruction, fibrosing mediastinitis, and chronic renal failure on dialysis.

The salt is a sulfate, phosphate, mesylate, bismesylate, tosylate, lactate, tartrate, malate, bis-acetate, citrate, or bishydrochloride salt, in some embodiments. In some embodiments, the compound of Structure 1, the tautomer of the compound, the pharmaceutically acceptable salt of the compound, the pharmaceutically acceptable salt of the tautomer, or the mixture thereof, covalently interacts with a receptor tyrosine kinase (RTK). In some embodiments, the RTK is PDGFR or c-Kit or both. In some embodiments, the PDGFR is selected from the group consisting of PDGFR-α, PDGFR-β, PDGFR-αα, PDGFR-ββ, and PDGFR-αβ.

In some embodiments, the compound of Structure 1 interacts with one or more of the PDGFR-α, PDGFR-β, PDGFR-αα, PDGFR-ββ, and/or the PDGFR-αβ amino acids selected from the group consisting of LYS627, VAL607, GLU644, MET648, HIS816, LEU809, ASP836, CYS814, ILE834, CYS835, PHE937, LYS634, VAL614, GLU651, MET655, HIS824, LEU817, ASP844, CYS822, ILE842, VAL658, ILE647, HIS816, ARG836, LYS634, GLU651, ALA632, HIS824, MET655, ARG825, CYS843, THR874, VAL607, ARG817, VAL815, LEU651, ILE657, THR681, ILE654, ARG825, ASP826, LEU658, LEU825, PHE837, LEU658, HIS824, CYS814, ILE654, ASP844, ILE842, and CYS843.

The compound of Structure 1 is a compound of Structure 3, 4, 5, 6, 7, 8, 9, 10, 11 or 12, in some embodiments, as noted above in Chart A of the Summary. In some embodiments, the compound of Structure 1 is a compound of Structure 13, 14, 15, 16, 17, 18, 19, 20, 21, 22, 23, 24, 25, 26, 27, 28, 29, 30, 31, 32, 33 or 34, as noted in Chart B. In illustrative embodiments, the treatment methods result in one or more of improved exercise capacity, improved functional class, less shortness of breath, decreased hospitalization, decreased need for lung transplantation, decreased need for atrial septostomy, and increased longevity or overall survival. In some embodiments, the improved exercise capacity is an increased 6 minute walk distance. In suitable embodiments, improved functional class is an improvement from class IV to class III, II or I, or an improvement from class III to class II or I, or an improvement form class II to class I.

### BRIEF DESCRIPTION OF THE FIGURES

FIG. 1 is a fluorescence image of frozen murine lung sections (right upper, middle, and lower lobes) after 2 min of PK10453 and IR780 tracer inhalation. Image acquisition occurred at 800 nm (green), i.e., the wavelength of IR780 detection, while image acquisition at 700 nm (red) represents tissue autofluorescence. Digital ruler intervals are show (1 cm).

FIG. 2 shows graphic data for intravenous (IV) and inhaled (INH) PK10453. FIG. 2A is a pharmacokinetic (PK) graph of IV administered PK10453 with concentrations in the lungs and plasma as a function of time. FIG. 2B is a PK graph concerning INH administered PK10453 and associated concentrations in the lungs and plasma as a function of time.

FIG. 3 depicts the effect of PK10453 on right ventricle (RV) systolic pressure and RV hypertrophy in the MCT and MCT+PN model systems. FIG. 3A is a graph showing the effect of PK10453 on RV systolic pressure in the MCT model, where C (n=3), V (n=2), D2 (n=6), D4 (n=6), and D8 (n=5) respectively represent control, vehicle, 2 minute (min) exposure, 4 min exposure, and 8 min exposure times, for two weeks, three times daily. Asterisks (*) indicate p<0.001 and section symbols (§) indicate p<0.05. FIG. 3B is a graph showing the effect of PK10453 on RV hypertrophy in the MCT model, where inhalation treatments were initiated three weeks after administration of MCT. C, D2, D4, and D8 respectively represent controls, 2, 4, and 8 min exposure times, for two weeks three times daily. The asterisks (*) indicate p<0.001. FIG. 3C is a graph showing the effect of PK10453 on RV systolic pressure (RVSP) in the MCT+PN model following two weeks of treatment with PK10453, which began two weeks after administration of MCT. V (n=10), D4 (n=10), and D8 (n=6) respectively represent vehicle 4 min exposure, and 8 min exposure times, where asterisks (*) indicate p<0.001. FIG. 3D is a graph showing the effect of PK10453 on RV hypertrophy in the MCT+PN model following two weeks of treatment with PK10453, which began two weeks after administration of MCT.

FIG. 4. is a graph showing pulmonary artery systolic pressure measured over time in ambulatory subjects using the MCT+PN model system. V (n=5) and D4 (n=6) respectively represent vehicle and 4 min exposure to PK10453 three times daily. Asterisks (*) indicate p<0.001 and section symbols (§) indicate p<0.01.

FIG. 5 shows microscopic images of pulmonary arteriole hypertrophy and intraluminal cellular proliferation of PK10453 treated specimens. FIG. 5A shows a 25× objective microscope image (OMI) of neointimal lesions. FIG. 5B shows a 40×OMI of hypertrophied pulmonary arteriole in vehicle administered subjects. FIG. 5C shows a D4 25×OMI of patent vessels. FIG. 5D shows a 40×OMI of pulmonary arterioles with decreased hypertrophy using the MCT+PN model.

FIG. 6 is a graph showing that the lumen area:media area ratio is increase in the D4 (n=6) and D8 (n=5) treated groups compared to vehicle (n=6) in the MCT+PN model. Section symbol (§) indicates p=0.032 (D4 vs. V), symbol (1) indicates p=0.028 (D8 vs. D4), and asterisk (*) indicates p=0.00014 (D8 vs. V).

FIG. 7 depicts an immunohistochemical evaluation of samples using the MCT+PN model. FIG. 7A shows vehicle treated CD20 positive B cells in a cellular proliferative lesion within a pulmonary arteriole. FIG. 7B shows that vehicle treated T-cells are present to a lesser extent in pulmonary arteriole intraluminal lesions and perivascular infiltrates compared to B-cells. FIG. 7C shows that pSTAT3 localized to the nuclei of endothelial cells and perivascular cells in vehicle treated tissue. FIG. 7D shows the pSTAT3 nuclear signal in the lung from a subject treated with PK10453. FIG. 7E shows total STAT3 in vehicle treated subjects. FIG. 7F shows vehicle treated tissue stained for vWF, a marker of endothelial cells, which is predominantly located in the pulmonary arteriole lumen.

FIG. 8 depicts an immunohistochemical evaluation of PDGFR signaling in the MCT+PN model. FIG. 8A shows a vehicle treated image of PDGFR alpha staining using a 10× objective microscope. FIG. 8B shows a vehicle treated image of total PDGFR alpha using a 40× objective microscope. FIG. 8C shows a PK10453 treated image of PDGFR beta staining at 40×. FIG. 8D shows a vehicle treated image of total PDGFR beta staining at 40×. FIG. 8E shows PK10453 treated image of phospho-PDGFR beta at 40×. FIG. 8F shows vehicle treated image of phospho-PDGFR beta at 40×. FIG. 8G shows a vehicle treated image of PDGFR alpha staining with competing peptide. FIG. 8H shows a vehicle treated phospho-PDGFR beta staining with competing peptide.

FIG. 9 shows results from experiments employing the NanoPro Immunoassay lumogram for pSTAT3 and STAT3 in the MCT+PN model. FIG. 9A is a graph of the vehicle treated subjects. FIG. 9B is a graph of the PK10453 treated subjects. FIG. 9C shows a graph of PK10453 treatment, which decreased pSTAT3/STAT3 in the lungs of subjects using the MCT+PN model (n=4), where V represents vehicle, D4 represents 4 min exposure times three times daily, and D8 represent 8 min exposure times for two weeks three times daily. 3×/day for two weeks PK10453. Asterisks (*) indicate p=0.009 and (§) indicate p=0.024.

FIG. 10 shows the results from experiments using the Nanopro Immunoassay lumograms for phospho-Erk1/2 and total ERK1/2 in the MCT+PN model. FIG. 10A shows phoshoERK1/2 in vehicle treated subjects. FIG. 10B shows phosphoERK1/2 in PK10453 treated subjects. FIG. 10C shows total ERK1/2 and vehicle treated subjects. FIG. 10D shows total ERK1/2 in PK10453 treated subjects, where PK10453 decreased ppERK1/ERK1. FIG. 10E shows ppERK1/ERK1 in subjects as indicated. FIG. 10F shows pERK2/ERK2 as indicated. FIG. 10G shows ppERK2/ERK2 as indicated in the lungs. FIG. 10H shows pERK2/ERK2 as indicated in lungs. n=4 for each group, while V represents vehicle, D4 is 4 min exposures, 3 times daily, and D8 is 8 min exposure times of PK10453 for two weeks three times daily. Asterisks (*) indicate p<0.0005 and section symbols indicate §p=0.045.

FIG. 11 is a graphic representation of subject body weight in vehicle administered and PK10453 treated subjects, where squares indicate vehicle treated (n=10), triangles indicate PK10453 D4 group (n=10), and diamonds indicate PK10453 D8 group (n=6).

FIG. 12 is a graph representing PAC40 telemetry transmitter data from transmitters implanted in the abdominal aorta for monitoring systemic blood pressure for seven days in ambulatory MCT exposed subjects treated with vehicle (n=3) or PK10453 (n=3).

FIG. 13 shows molecular docking and modeling data. FIGS. 13A-B are Ramachandran plots of PDGF-alpha and PDGF-beta subunits of PDGF, respectively, where more than 93% of the residues fell within the most favored region, with no residues present in the disallowed region. FIG. 13C shows the structural alignment of the template molecule (c-Kit) and the PDGF models. The total backbone RMSD, as compared to the template structure, was 0.222 Å and 0.210 Å for PDGF-alpha and PDGF-beta subunits, respectively. The template is indicated with yellow, PDGF-alpha with red, and PDGF-beta with blue color. The coordinates of the models were transformed based on the template coordinates.

FIG. 14 shows the free energy of binding and estimated Ki calculated for two representative compounds. FIG. 14A shows molecular docking of PK10498 (Structure 18) with PDGFR-alpha, where the long arrow points to the “warhead” structure and the short arrow points to CYS814. The warhead is in suboptimal spatial orientation, where PK10498 free energy binding estimate −10.07 kcal/mol and Ki 41.79 nM. FIG. 14B shows molecular docking of PK10562 (Structure 30) with PDGFR-alpha. Proximity of the nitrile containing warhead in proximity to CYS814 is shown (oval encircles both CYS814 and warhead). The warhead is in optimal position to form a covalent bond with CYS814. PK10562 free energy binding estimate −10.37 kcal/mol; Ki 25 nM, while use of the CF₃ moiety improves binding energy thereby increases selectivity and specificity.

### DETAILED DESCRIPTION

The present disclosure relates to, inter alia, a novel class of compounds which function as kinase inhibitors. Likewise, methods for using such compounds in the prevention and treatment of disease conditions are disclosed herein. The present disclosure further relates to pharmaceutical formulations of the compounds, which possess prophylactic and/or therapeutic indications for subjects in need of kinase inhibitors, e.g., patients afflicted with vascular disease, proliferative disorders, cancers, and related diseases or conditions, as further detailed below. The definitions of certain terms as used in this specification are provided below. Unless defined otherwise, all technical and scientific terms used herein generally have the same meaning as commonly understood by one of ordinary skill in the art to which this invention belongs. As used in this specification and the appended claims, the singular forms “a”, “an” and “the” include plural referents unless content clearly dictates otherwise.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the enumerated value.

As used herein, the “administration” of an agent or drug, e.g., one or more kinase inhibitor compounds, to a subject or subjects includes any route of introducing or delivering to a subject a compound to perform its intended function. Administration can be carried out by any suitable route, including orally, intranasally, by inhalation, parenterally (intravenously, intramuscularly, intraperitoneally, or subcutaneously), rectally, or topically. Administration includes self-administration and the administration by another. It is also to be appreciated that the various modes of treatment or prevention of medical conditions as described are intended to mean “substantial”, which includes total but also less than total treatment/prevention, and wherein some biologically or medically relevant result is achieved.

As used herein, the terms “assessing,” “assaying,” “determining,” and “measuring” are used interchangeably and include both quantitative and qualitative determinations. These terms refer to any form of measurement, and include determining if a characteristic, trait, or feature is present or not. Assessing may be relative or absolute. “Assessing the presence of” includes determining the amount of something present and/or absent.

As used herein, the term “clinical factors” refers to any data that a medical practitioner may consider in determining a diagnosis, prognosis, or therapeutic regimen for treating or preventing a disease or diseases. Such factors include, but are not limited to, the patient's medical history, a physical examination of the patient, complete blood count, examination of blood cells or bone marrow cells, cytogenetics, pulmonary health, vascular indications of disease, and immunophenotyping of cells.

As used herein, the terms “comparable” or “corresponding” in the context of comparing two or more samples, responses to treatment, or drugs, refer to the same type of sample, response, treatment, and drug respectively used in the comparison. For example, the phosphorylation state or level of STAT3 in a sample can be compared to the phosphorylation state or level in another sample. In some embodiments, comparable samples may be obtained from the same individual at different times. In other embodiments, comparable samples may be obtained from different individuals, e.g., a patient and a healthy individual. In general, comparable samples are normalized by a common factor for control purposes.

As used herein, the term “composition” refers to a product with specified ingredients in the specified amounts, as well as any product which results, directly or indirectly, from combination of the specified ingredients in the specified amounts.

As used herein, the term “diagnosis” means detecting a disease or disorder or determining the stage or degree of a disease or disorder. Typically, a diagnosis of a disease or disorder is based on the evaluation of one or more factors and/or symptoms that are indicative of the disease. That is, a diagnosis can be made based on the presence, absence or amount of a factor which is indicative of presence or absence of the disease or condition. Each factor or symptom that is considered to be indicative for the diagnosis of a particular disease does not need be exclusively related to the particular disease, i.e., there may be differential diagnoses that can be inferred from a diagnostic factor or symptom. Likewise, there may be instances where a factor or symptom that is indicative of a particular disease is present in an individual that does not have the particular disease. The term “diagnosis” also encompasses determining the therapeutic effect of a drug therapy, or predicting the pattern of response to a drug therapy. The diagnostic methods may be used independently, or in combination with other diagnosing and/or staging methods known in the art for a particular disease, e.g., PAH.

As used herein, the terms “diseased mediated by” or “disease associated with” one or more kinases refer to a disease or condition that directly or indirectly results from, or are aggravated by, kinase activity, such as, e.g., aberrant kinase activity. In particular, kinase activity associated with, for example, cell division cycle 2 kinase (Cdc2 kinase), c-Kit, c-ABL, p60src, VEGFR3, PDGF, PDGFRα, PDGFRβ, PDGFR-αα, PDGFR-ββ, PDGFR-αβ, FGFR3, FLT-3, FYN oncogene kinase related to SRC, FGR, YES (Fyn), lymphocyte-specific protein tyrosine kinase (Lck), tyrosine kinase with Ig and EGF homology domains (Tie-2), FMS (CSF-IR), KDR, EphA2, EphA3, EphA8, FLT1, FLT4, HCK, PTK5, RET, SYK, DDR1, DDR2, glycogen synthase kinase 3 (GSK-3), cyclin dependent kinase 2 (Cdk2), cyclin dependent kinase 4 (Cdk4), MEK1, NEK-2, CHK2, CK1ε, Raf, checkpoint kinase 1 (CHK1), ribosomal S6 kinase 2 (Rsk2), and PAR-1, and combinations thereof, are activities that may precipitate various diseases or conditions described herein.

As used herein, the terms “drug,” “compound,” “active agent,” “agent,” “actives,” “pharmaceutical composition,” “pharmaceutical formulation,” and “pharmacologically active agent” are used interchangeably and refer to any chemical compound, complex or composition, charged or uncharged, that is suitable for administration and that has a beneficial biological effect, suitably a therapeutic effect in the treatment of a disease or abnormal physiological condition, although the effect may also be prophylactic in nature. The terms also encompass pharmaceutically acceptable, pharmacologically active derivatives of those active agents specifically mentioned herein, including, but not limited to, salts, esters, amides, prodrugs, active metabolites, analogs, and the like. When the terms “active agent,” “pharmacologically active agent,” and “API” (active pharmaceutical ingredient) are used, then, or when a particular active agent is specifically identified, it is to be understood that applicants intend to include the active agent per se as well as pharmaceutically acceptable and/or active salts, esters, amides, prodrugs, conjugates, metabolites, analogs, etc.

As used herein, the terms “effective amount” or “pharmaceutically effective amount” or “therapeutically effective amount” of a composition, is a quantity sufficient to achieve a desired therapeutic and/or prophylactic effect, e.g., an amount which results in the prevention of, or a decrease in, the symptoms associated with a disease that is being treated. The amount of a composition of the invention administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The compositions of the present invention can also be administered in combination with one or more additional therapeutic compounds.

As used herein, the terms “irreversible” or “irreversibly” when referring to a kinase inhibitor means an inhibitor of the activity of a kinase, tyrosine kinase, and/or receptor tyrosine kinase, which is covalently (permanently), bound or associated with such a kinase.

As used herein, the term “neoplastic disease” refers to cancers of any kind and origin and precursor stages thereof. Accordingly, the term “neoplastic disease” includes the subject matter identified by the terms “neoplasia”, “neoplasm”, “cancer”, “pre-cancer” or “tumor.” A neoplastic disease is generally manifest by abnormal cell division resulting in an abnormal level of a particular cell population. Likewise, because the monoclonal expansion of endothelial cells may refer to a “neoplasm” of the pulmonary arteriolar endothelial cells, PAH is also encompassed within the foregoing terms. The abnormal cell division underlying a neoplastic disease, moreover, is typically inherent in the cells and not a normal physiological response to infection or inflammation. In some embodiments, neoplastic diseases for diagnosis using methods provided herein include carcinoma. By “carcinoma,” it is meant a benign or malignant epithelial tumor and includes, but is not limited to, the following carcinomas: hepatocellular, breast, prostate, non-small cell lung, colon, CNS, melanoma, ovarian, or renal, and the like.

As used herein, the term “pharmaceutically acceptable salt” includes a salt with an inorganic base, organic base, inorganic acid, organic acid, or basic or acidic amino acid. As salts of inorganic bases, the invention includes, for example, alkali metals such as sodium or potassium; alkaline earth metals such as calcium and magnesium or aluminum; and ammonia. As salts of organic bases, the invention includes, for example, trimethylamine, triethylamine, pyridine, picoline, ethanolamine, diethanolamine, and triethanolamine. As salts of inorganic acids, the instant invention includes, for example, hydrochloric acid, hydroboric acid, nitric acid, sulfuric acid, and phosphoric acid. As salts of organic acids, the instant invention includes, for example, formic acid, acetic acid, trifluoroacetic acid, fumaric acid, oxalic acid, tartaric acid, maleic acid, lactic acid, citric acid, succinic acid, malic acid, methanesulfonic acid, benzenesulfonic acid, and p-toluenesulfonic acid. As salts of basic amino acids, the instant invention includes, for example, arginine, lysine and ornithine. Acidic amino acids include, for example, aspartic acid and glutamic acid.

As used herein, the term “reference level” refers to a level of a substance which may be of interest for comparative purposes. In some embodiments, a reference level may be a specified composition dosage as an average of the dose level from samples taken from a control subject. In other embodiments, the reference level may be the level in the same subject at a different time, e.g., a time course of administering the composition, such as the level determined at 2, 4, 6, 8, and 10 minutes (min), etc.

As used herein, the terms “sample” or “test sample” refer to any liquid or solid material containing collected from a subject. In suitable embodiments, a test sample is obtained from a biological source, i.e., a “biological sample,” such as cells in culture or a tissue sample from an animal, most preferably, a murine subject, mammal or human subject.

As used herein, the terms “subject” or “individual,” refer to a mammal, such as a mouse, rat, or human, but can also be another animal such as a domestic animal, e.g., a dog, cat, or the like, a farm animal, e.g., a cow, a sheep, a pig, a horse, or the like, or a laboratory animal, e.g., a monkey, a rat, a mouse, a rabbit, a guinea pig, or the like. The term “patient” refers to a “subject” who is, or is suspected to be, afflicted with a disease.

As used herein, the terms “treating” or “treatment” or “alleviation” refer to both therapeutic treatment and prophylactic or preventative measures, wherein the objective is to prevent or slow down (lessen) the targeted pathologic condition or disorder. A subject is successfully “treated” for a disorder if, after receiving a therapeutic agent according to the methods of the present invention, the subject shows observable and/or measurable reduction in or absence of one or more signs and symptoms of a particular disease or condition.

As used herein, reference to a certain element such as “hydrogen” or “H” is meant to include all isotopes of that element. For example, if an R group is defined to include hydrogen or H, it also includes deuterium and tritium.

As used herein, the term “unsubstituted alkyl” refers to alkyl groups that do not contain heteroatoms. Thus the phrase includes straight chain alkyl groups such as methyl, ethyl, propyl, butyl, pentyl, hexyl, heptyl, octyl, nonyl, decyl, undecyl, dodecyl and the like. The phrase also includes branched chain isomers of straight chain alkyl groups, including but not limited to, the following which are provided by way of example: —CH(CH₃)₂, —CH(CH₃)(CH₂CH₃), —CH(CH₂CH₃)₂, —C(CH₃)₃, —C(CH₂CH₃)₃, —CH₂CH(CH₃)₂, —CH₂CH(CH₃)(CH₂CH₃), —CH₂CH(CH₂CH₃)₂, —CH₂C(CH₃)₃, —CH₂C(CH₂CH₃)₃, —CH(CH₃)CH(CH₃)(CH₂CH₃), —CH₂CH₂CH(CH₃)₂, —CH₂CH₂CH(CH₃)(CH₂CH₃), —CH₂CH₂CH(CH₂CH₃)₂, —CH₂CH₂C(CH₃)₃, —CH₂CH₂C(CH₂CH₃)₃, —CH(CH₃)CH₂CH(CH₃)₂, —CH(CH₃)CH(CH₃)CH(CH₃)₂, —CH(CH₂CH₃)CH(CH₃)CH(CH₃)(CH₂CH₃), and others. The phrase also includes cyclic alkyl groups such as cycloalkyl groups such as cyclopropyl, cyclobutyl, cyclopentyl, cyclohexyl, cycloheptyl, and cyclooctyl and such rings substituted with straight and branched chain alkyl groups as defined above. The phrase also includes polycyclic alkyl groups such as, but not limited to, adamantyl norbornyl, and bicyclo[2.2.2]octyl and such rings substituted with straight and branched chain alkyl groups as defined above. Thus, the phrase unsubstituted alkyl groups includes primary alkyl groups, secondary alkyl groups, and tertiary alkyl groups. Unsubstituted alkyl groups may be bonded to one or more carbon atom(s), oxygen atom(s), nitrogen atom(s), and/or sulfur atom(s) in the parent compound. Preferred unsubstituted alkyl groups include straight and branched chain alkyl groups and cyclic alkyl groups having 1 to 20 carbon atoms. More preferred such unsubstituted alkyl groups have from 1 to 10 carbon atoms while even more preferred such groups have from 1 to 5 carbon atoms. In some embodiments, unsubstituted alkyl groups include straight and branched chain alkyl groups having from 1 to 3 carbon atoms and include methyl, ethyl, propyl, and —CH(CH₃)₂.

As used herein, the term “substituted alkyl” refers to an unsubstituted alkyl group as defined above in which one or more bonds to a carbon(s) or hydrogen(s) are replaced by a bond to non-hydrogen and non-carbon atoms such as, but not limited to, a halogen atom in halides such as F, Cl, Br, and I; an oxygen atom in groups such as hydroxyl groups, alkoxy groups, aryloxy groups, and ester groups; a sulfur atom in groups such as thiol groups, alkyl and aryl sulfide groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as in trialkylsilyl groups, dialkylarylsilyl groups, alkyldiarylsilyl groups, and triarylsilyl groups; and other heteroatoms in various other groups. Substituted alkyl groups also include groups in which one or more bonds to a carbon(s) or hydrogen(s) atom is replaced by a bond to a heteroatom such as oxygen in carbonyl, carboxyl, and ester groups; nitrogen in groups such as imines, oximes, hydrazones, and nitriles. In suitable embodiments, substituted alkyl groups include, among others, alkyl groups in which one or more bonds to a carbon or hydrogen atom is/are replaced by one or more bonds to fluorine atoms. One example of a substituted alkyl group is the trifluoromethyl group and other alkyl groups that contain the trifluoromethyl group. Other alkyl groups include those in which one or more bonds to a carbon or hydrogen atom is replaced by a bond to an oxygen atom such that the substituted alkyl group contains a hydroxyl, alkoxy, aryloxy group, or heterocyclyloxy group. Still other alkyl groups include alkyl groups that have an amine, alkylamine, dialkylamine, arylamine, (alkyl)(aryl)amine, diarylamine, heterocyclylamine, (alkyl)(heterocyclyl)amine, (aryl)(heterocyclyl)amine, or diheterocyclylamine group.

As used herein, the term “unsubstituted aryl” refers to aryl groups that do not contain heteroatoms. Thus the term includes, but is not limited to, groups such as phenyl, biphenyl, anthracenyl, naphthenyl by way of example. Although the phrase “unsubstituted aryl” includes groups containing condensed rings such as naphthalene, it does not include aryl groups that have other groups such as alkyl or halo groups bonded to one of the ring members, as aryl groups such as tolyl are considered herein to be substituted aryl groups as described below. Unsubstituted aryl groups may be bonded to one or more carbon atom(s), oxygen atom(s), nitrogen atom(s), and/or sulfur atom(s).

As used herein, the term “substituted aryl group” has the same meaning with respect to unsubstituted aryl groups that substituted alkyl groups had with respect to unsubstituted alkyl groups. However, a substituted aryl group also includes aryl groups in which one of the aromatic carbons is bonded to one of the non-carbon or non-hydrogen atoms described above and also includes aryl groups in which one or more aromatic carbons of the aryl group is bonded to a substituted and/or unsubstituted alkyl, alkenyl, or alkynyl group as defined herein. This includes bonding arrangements in which two carbon atoms of an aryl group are bonded to two atoms of an alkyl, alkenyl, or alkynyl group to define a fused ring system (e.g. dihydronaphthyl or tetrahydronaphthyl). Thus, the term “substituted aryl” includes, but is not limited to, tolyl and hydroxyphenyl, among others.

As used herein, the term “unsubstituted alkenyl” refers to straight and branched chain and cyclic groups such as those described with respect to unsubstituted alkyl groups as defined above, except that at least one double bond exists between two carbon atoms. Examples include, but are not limited to vinyl, —CH═C(H)(CH₃), —CH═C(CH₃)₂, —C(CH₃)═C(H)₂, —C(CH₃)═C(H)(CH₃), —C(CH₂CH₃)═CH₂, cyclohexenyl, cyclopentenyl, cyclohexadienyl, butadienyl, pentadienyl, and hexadienyl among others.

As used herein, the term “substituted alkenyl” has the same meaning with respect to unsubstituted alkenyl groups that substituted alkyl groups had with respect to unsubstituted alkyl groups. A substituted alkenyl group includes alkenyl groups in which a non-carbon or non-hydrogen atom is bonded to a carbon double bonded to another carbon and those in which one of the non-carbon or non-hydrogen atoms is bonded to a carbon not in a C═C.

As used herein, the term “unsubstituted alkynyl” refers to straight and branched chain groups such as those described with respect to unsubstituted alkyl groups as defined above, except that at least one triple bond exists between two carbon atoms. Examples include, but are not limited to, —C≡C(H), —C≡C(CH₃), —C≡C(CH₂CH₃), —C(H₂)C≡C(H), —C(H)₂C≡C(CH₃), and —C(H)₂C≡C(CH₂CH₃), among others.

As used herein, the term “substituted alkynyl” has the same meaning with respect to unsubstituted alkynyl groups that substituted alkyl groups had with respect to unsubstituted alkyl groups. A substituted alkynyl group includes alkynyl groups in which a non-carbon or non-hydrogen atom is bonded to a carbon triple bonded to another carbon and those in which a non-carbon or non-hydrogen atom is bonded to a carbon not involved in a C≡C bond.

As used herein, the term “unsubstituted aralkyl” refers to unsubstituted alkyl groups as defined above in which a hydrogen or carbon bond of the unsubstituted alkyl group is replaced with a bond to an aryl group as defined above. For example, methyl (—CH₃) is an unsubstituted alkyl group. If a hydrogen atom of the methyl group is replaced by a bond to a phenyl group, such as if the carbon of the methyl were bonded to a carbon of benzene, then the compound is an unsubstituted aralkyl group, i.e., a benzyl group. Thus, the term includes, but is not limited to, groups such as benzyl, diphenylmethyl, and 1-phenylethyl (—CH(C₆H₅)(CH₃)), among others.

As used herein, the term “substituted aralkyl” has the same meaning with respect to unsubstituted aralkyl groups that substituted aryl groups had with respect to unsubstituted aryl groups. However, a substituted aralkyl group also includes groups in which a carbon or hydrogen bond of the alkyl part of the group is replaced by a bond to a non-carbon or a non-hydrogen atom. Examples of substituted aralkyl groups include, but are not limited to, —CH₂C(═O)(C₆H₅), and —CH₂(2-methylphenyl), among others.

As used herein, the term “unsubstituted heterocyclyl” refers to both aromatic and nonaromatic ring compounds including monocyclic, bicyclic, and polycyclic ring compounds such as, but not limited to, quinuclidyl, containing 3 or more ring members of which one or more is a heteroatom such as, but not limited to N, O, and S. Examples of heterocyclyl groups include, but are not limited to: unsaturated 3 to 8 membered rings containing 1 to 4 nitrogen atoms such as, but not limited to pyrrolyl, pyrrolinyl, imidazolyl, pyrazolyl, pyridinyl, dihydropyridinyl, pyrimidyl, pyrazinyl, pyridazinyl, triazolyl, e.g., 4H-1,2,4-triazolyl, 1H-1,2,3-triazolyl, 2H-1,2,3-triazolyl etc., tetrazolyl, e.g., 1H-tetrazolyl, 2H tetrazolyl, etc.); saturated 3 to 8 membered rings containing 1 to 4 nitrogen atoms such as, but not limited to, pyrrolidinyl, imidazolidinyl, piperidinyl, piperazinyl; condensed unsaturated heterocyclic groups containing 1 to 4 nitrogen atoms such as, but not limited to, indolyl, isoindolyl, indolinyl, indolizinyl, benzimidazolyl, quinolyl, isoquinolyl, indazolyl, benzotriazolyl; unsaturated 3 to 8 membered rings containing 1 to 2 oxygen atoms and 1 to 3 nitrogen atoms such as, but not limited to, oxazolyl, isoxazolyl, oxadiazolyl, e.g., 1,2,4-oxadiazolyl, 1,3,4-oxadiazolyl, 1,2,5-oxadiazolyl, etc.; saturated 3 to 8 membered rings containing 1 to 2 oxygen atoms and 1 to 3 nitrogen atoms such as, but not limited to, morpholinyl; unsaturated condensed heterocyclic groups containing 1 to 2 oxygen atoms and 1 to 3 nitrogen atoms, for example, benzoxazolyl, benzoxadiazolyl, benzoxazinyl, e.g. 2H-1,4-benzoxazinyl etc.); unsaturated 3 to 8 membered rings containing 1 to 3 sulfur atoms and 1 to 3 nitrogen atoms such as, but not limited to, thiazolyl, isothiazolyl, thiadiazolyl, e.g., 1,2,3-thiadiazolyl, 1,2,4-thiadiazolyl, 1,3,4-thiadiazolyl, 1,2,5-thiadiazolyl, etc.; saturated 3 to 8 membered rings containing 1 to 2 sulfur atoms and 1 to 3 nitrogen atoms such as, but not limited to, thiazolodinyl; saturated and unsaturated 3 to 8 membered rings containing 1 to 2 sulfur atoms such as, but not limited to, thienyl, dihydrodithiinyl, dihydrodithionyl, tetrahydrothiophene, tetrahydrothiopyran; unsaturated condensed heterocyclic rings containing 1 to 2 sulfur atoms and 1 to 3 nitrogen atoms such as, but not limited to, benzothiazolyl, benzothiadiazolyl, benzothiazinyl (e.g. 2H-1,4-benzothiazinyl, etc.), dihydrobenzothiazinyl, e.g., 2H-3,4-dihydrobenzothiazinyl, etc., unsaturated 3 to 8 membered rings containing oxygen atoms such as, but not limited to furyl; unsaturated condensed heterocyclic rings containing 1 to 2 oxygen atoms such as benzodioxolyl, e.g., 1,3-benzodioxoyl, etc.; unsaturated 3 to 8 membered rings containing an oxygen atom and 1 to 2 sulfur atoms such as, but not limited to, dihydrooxathiinyl; saturated 3 to 8 membered rings containing 1 to 2 oxygen atoms and 1 to 2 sulfur atoms such as 1,4-oxathiane; unsaturated condensed rings containing 1 to 2 sulfur atoms such as benzothienyl, benzodithiinyl; and unsaturated condensed heterocyclic rings containing an oxygen atom and 1 to 2 oxygen atoms such as benzoxathiinyl. Heterocyclyl group also include those described above in which one or more S atoms in the ring is double-bonded to one or two oxygen atoms (sulfoxides and sulfones). For example, heterocyclyl groups include tetrahydrothiophene oxide and tetrahydrothiophene 1,1-dioxide. Preferred heterocyclyl groups contain 5 or 6 ring members. More preferred heterocyclyl groups include morpholine, piperazine, piperidine, pyrrolidine, imidazole, pyrazole, 1,2,3-triazole, 1,2,4-triazole, tetrazole, thiophene, thiomorpholine, thiomorpholine in which the S atom of the thiomorpholine is bonded to one or more O atoms, pyrrole, homopiperazine, oxazolidin-2-one, pyrrolidin-2-one, oxazole, quinuclidine, thiazole, isoxazole, furan, and tetrahydrofuran.

As used herein, the term “substituted heterocyclyl” refers to an unsubstituted heterocyclyl group as defined above in which one or more of the ring members is bonded to a non-hydrogen atom such as described above with respect to substituted alkyl groups and substituted aryl groups. Examples, include, but are not limited to, 2-methylbenzimidazolyl, 5-methylbenzimidazolyl, 5-chlorobenzthiazolyl, N-alkyl piperazinyl groups such as 1-methyl piperazinyl, piperazine-N-oxide, N-alkyl piperazine N-oxides, 2-phenoxy-thiophene, and 2-chloropyridinyl among others. In addition, substituted heterocyclyl groups also include heterocyclyl groups in which the bond to the non-hydrogen atom is a bond to a carbon atom that is part of a substituted and unsubstituted aryl, substituted and unsubstituted aralkyl, or unsubstituted heterocyclyl group. Examples include but are not limited to 1-benzylpiperidinyl, 3-phenythiomorpholinyl, 3-(pyrrolidin-1-yl)-pyrrolidinyl, and 4-(piperidin-1-yl)-piperidinyl. Groups such as N-alkyl substituted piperazine groups such as N-methyl piperazine, substituted morpholine groups, and piperazine N-oxide groups such as piperazine N-oxide and N-alkyl piperazine N-oxides are examples of some substituted heterocyclyl groups. Groups such as substituted piperazine groups such as N-alkyl substituted piperazine groups such as N-methyl piperazine and the like, substituted morpholine groups, piperazine N-oxide groups, and N-alkyl piperazine N-oxide groups are examples of some substituted heterocyclyl groups that are suited for various “R” groups.

As used herein, the term “unsubstituted heterocyclylalkyl” refers to unsubstituted alkyl groups as defined above in which a hydrogen or carbon bond of the unsubstituted alkyl group is replaced with a bond to a heterocyclyl group as defined above. For example, methyl (—CH₃) is an unsubstituted alkyl group. If a hydrogen atom of the methyl group is replaced by a bond to a heterocyclyl group, such as if the carbon of the methyl were bonded to carbon 2 of pyridine (one of the carbons bonded to the N of the pyridine) or carbons 3 or 4 of the pyridine, then the compound is an unsubstituted heterocyclylalkyl group.

As used herein, the term “substituted heterocyclylalkyl” has the same meaning with respect to unsubstituted heterocyclylalkyl groups that substituted aralkyl groups had with respect to unsubstituted aralkyl groups. However, a substituted heterocyclylalkyl group also includes groups in which a non-hydrogen atom is bonded to a heteroatom in the heterocyclyl group of the heterocyclylalkyl group such as, but not limited to, a nitrogen atom in the piperidine ring of a piperidinylalkyl group. In addition, a substituted heterocyclylalkyl group also includes groups in which a carbon bond or a hydrogen bond of the alkyl part of the group is replaced by a bond to a substituted/unsubstituted aryl or aralkyl group.

As used herein, the term “unsubstituted alkylaminoalkyl” refers to an unsubstituted alkyl group as defined above in which a carbon or hydrogen bond is replaced by a bond to a nitrogen atom that is bonded to a hydrogen atom and an unsubstituted alkyl group as defined above. For example, methyl (—CH₃) is an unsubstituted alkyl group. If a hydrogen atom of the methyl group is replaced by a bond to a nitrogen atom that is bonded to a hydrogen atom and an ethyl group, then the resulting compound is —CH₂—N(H)(CH₂CH₃) which is an unsubstituted alkylaminoalkyl group.

As used herein, the term “substituted alkylaminoalkyl” refers to an unsubstituted alkylaminoalkyl group as defined above except where one or more bonds to a carbon or hydrogen atom in one or both of the alkyl groups is replaced by a bond to a non-carbon or non-hydrogen atom as described above with respect to substituted alkyl groups except that the bond to the nitrogen atom in all alkylaminoalkyl groups does not by itself qualify all alkylaminoalkyl groups as being substituted.

As used herein, the term “unsubstituted alkoxy” refers to a hydroxyl group (—OH) in which the bond to the hydrogen atom is replaced by a bond to a carbon atom of an otherwise unsubstituted alkyl group as defined above. As used herein, the term “substituted alkoxy” refers to a hydroxyl group (—OH) in which the bond to the hydrogen atom is replaced by a bond to a carbon atom of an otherwise substituted alkyl group as defined above.

As used herein, the term “unsubstituted heterocyclyloxy” refers to a hydroxyl group (—OH) in which the bond to the hydrogen atom is replaced by a bond to a ring atom of an otherwise unsubstituted heterocyclyl group as defined above. As used herein, the term “substituted heterocyclyloxy” refers to a hydroxyl group (—OH) in which the bond to the H atom is replaced by a bond to a ring atom of an otherwise substituted heterocyclyl group.

As used herein, the term “unsubstituted heterocyclyloxyalkyl” refers to an unsubstituted alkyl group as defined above in which a carbon bond or hydrogen bond is replaced by a bond to an oxygen atom which is bonded to an unsubstituted heterocyclyl group as defined above. As used herein, the term “substituted heterocyclyloxyalkyl” refers to an unsubstituted heterocyclyloxyalkyl group as defined above in which a bond to a carbon or hydrogen group of the alkyl group of the heterocyclyloxyalkyl group is bonded to a non-carbon and non-hydrogen atom as described above with respect to substituted alkyl groups or in which the heterocyclyl group of the heterocyclyloxyalkyl group is a substituted heterocyclyl group as defined above.

As used herein, the term “unsubstituted heterocyclylalkoxy” refers to an unsubstituted alkyl group as defined above in which a carbon bond or hydrogen bond is replaced by a bond to an oxygen atom which is bonded to the parent compound, and in which another carbon or hydrogen bond of the unsubstituted alkyl group is bonded to an unsubstituted heterocyclyl group as defined above. As used herein, the term “substituted heterocyclylalkoxy” refers to an unsubstituted heterocyclylalkoxy group as defined above in which a bond to a carbon or hydrogen group of the alkyl group of the heterocyclylalkoxy group is bonded to a non-carbon and non-hydrogen atom as described above with respect to substituted alkyl groups or in which the heterocyclyl group of the heterocyclylalkoxy group is a substituted heterocyclyl group as defined above. Further, a substituted heterocyclylalkoxy group also includes groups in which a carbon bond or a hydrogen bond to the alkyl moiety of the group may be substituted with one or more additional heterocycles.

As used herein, the term “unsubstituted arylaminoalkyl” refers to an unsubstituted alkyl group as defined above in which a carbon bond or hydrogen bond is replaced by a bond to a nitrogen atom which is bonded to at least one unsubstituted aryl group as defined above.

As used herein, the term “substituted arylaminoalkyl” refers to an unsubstituted arylaminoalkyl group as defined above except where either the alkyl group of the arylaminoalkyl group is a substituted alkyl group as defined above or the aryl group of the arylaminoalkyl group is a substituted aryl group except that the bonds to the nitrogen atom in all arylaminoalkyl groups does not by itself qualify all arylaminoalkyl groups as being substituted. However, substituted arylaminoalkyl groups does include groups in which the hydrogen bonded to the nitrogen atom of the group is replaced with a non-C and non-H atom.

As used herein, the term “unsubstituted heterocyclylaminoalkyl” refers to an unsubstituted alkyl group as defined above in which a carbon or hydrogen bond is replaced by a bond to a nitrogen atom which is bonded to at least one unsubstituted heterocyclyl group as defined above. As used herein, the term “substituted heterocyclylaminoalkyl” refers to unsubstituted heterocyclylaminoalkyl groups as defined above in which the heterocyclyl group is a substituted heterocyclyl group as defined above and/or the alkyl group is a substituted alkyl group as defined above.

As used herein, the term “unsubstituted alkylaminoalkoxy” refers to an unsubstituted alkyl group as defined above in which a carbon or hydrogen bond is replaced by a bond to an oxygen atom which is bonded to the parent compound and in which another carbon or hydrogen bond of the unsubstituted alkyl group is bonded to a nitrogen atom which is bonded to a hydrogen atom and an unsubstituted alkyl group as defined above. As used herein, the term “substituted alkylaminoalkoxy” refers to unsubstituted alkylaminoalkoxy groups as defined above in which a bond to a carbon or hydrogen atom of the alkyl group bonded to the oxygen atom which is bonded to the parent compound is replaced by one or more bonds to a non-carbon and non-hydrogen atoms as discussed above with respect to substituted alkyl groups and/or if the hydrogen bonded to the amino group is bonded to a non-carbon and non-hydrogen atom and/or if the alkyl group bonded to the nitrogen of the amine is bonded to a non-carbon and non-hydrogen atom as described above with respect to substituted alkyl groups.

As used herein, the term “protected” with respect to hydroxyl groups, amine groups, and sulfhydryl groups refers to forms of these functionalities which are protected from undesirable reaction with a protecting group known to those skilled in the art such as those set forth in Protective Groups in Organic Synthesis, Greene, T. W.; Wuts, P. G. M., John Wiley & Sons, New York, N.Y., (3rd Edition, 1999), which can be added or removed using the procedures set forth therein. Examples of protected hydroxyl groups include, but are not limited to, silyl ethers such as those obtained by reaction of a hydroxyl group with a reagent such as, but not limited to, t-butyldimethyl-chlorosilane, trimethylchlorosilane, triisopropylchlorosilane, triethylchlorosilane; substituted methyl and ethyl ethers such as, but not limited to methoxymethyl ether, methythiomethyl ether, benzyloxymethyl ether, t-butoxymethyl ether, 2-methoxyethoxymethyl ether, tetrahydropyranyl ethers, 1-ethoxyethyl ether, allyl ether, benzyl ether; esters such as, but not limited to, benzoylformate, formate, acetate, trichloroacetate, and trifluoracetate. Examples of protected amine groups include, but are not limited to, amides such as, formamide, acetamide, trifluoroacetamide, and benzamide; imides, such as phthalimide, and dithiosuccinimide; and others. Examples of protected sulfhydryl groups include, but are not limited to, thioethers such as S-benzyl thioether, and S-4-picolyl thioether; substituted S-methyl derivatives such as hemithio, dithio and aminothio acetals, among others.

### Overview

Various compounds have been found useful in treating certain diseases such as, e.g., cancer. For example, Gleevec® (imatinib mesylate or “imatinib”) is a compound that has shown efficacy in treating chronic myeloid leukemia (CML) and gastrointestinal stromal tumors (GIST). Other experimental drugs include sorafenib and PNU-166196 for the respective treatment of renal cell carcinoma and leukemia. Although significant advances have been made in the development of pharmaceutical compositions for treating certain cancers, new compounds, compositions, methods of treatment, and model systems for developing drugs are required for preventing and/or treating cancer and other diseases, e.g., pulmonary-vascular disease such as pulmonary arterial hypertension (PAH). For example, imatinib was developed using an in vivo murine MCT model system, which is an imperfect system concerning preclinical drug candidate efficacy assessment at least because it is unreliable with respect to expressing certain human disease phenotypes, e.g., the development of neointimal and/or plexiform lesions associated with PAH. Cool et al., “Pathogenesis and evolution of plexiform lesions in pulmonary hypertension associated with scleroderma and human immunodeficiency virus infection.” Hum Pathol. 28:434-442 (1997). Therefore, examining the effects of kinase inhibitors in more aggressive models presenting human disease phenotypes is essential for more accurately reflecting the pathology of the human disease and, consequently, the development of the next generation of compounds and compositions for effectively treating human disease.

The present inventors have employed such a model. As further detailed below, the present inventors performed efficacy studies using a rat monocrotaline (MCT) plus pneumonectomy (PN) model system (MCT+PN). This model imparts neointimal and/or plexiform lesions characteristic of human disease, e.g., PAH. To this end, for example, the pathologic signature of PAH consists of concentric and plexiform lesions in small precapillary pulmonary arterioles. See Cool et al. (1997); and Tuder et al., “Plexiform lesion in severe pulmonary hypertension: association with glomeruloid lesion.” Am J Pathol 159:382-383 (2001). Concentric lesions arise from the proliferation of neointimal cells, which occlude the vessel lumen. It has been reported that these concentric obstructive neointimal lesions are composed of myofibroblasts and/or endothelial cells. See, e.g., Yi et al., Am J Respir Crit Care Med 162:1577-1586 (2000).

In addition, perivascular infiltrates, consisting of T cells, B cells, and macrophages, have been found in plexogenic PAH. See Sakagami, Adv Drug Deliv Rev 58:1030-1060 (2006). Plexiform lesions, moreover, are characterized by disorganized vascular channels that stain for endothelial cell markers, and such lesions in lung samples from patients with idiopathic and/or primary PAH consist of a monoclonal expansion of endothelial cells. Lee et al., J Clin Invest 101:927-934 (1998). As such, PAH of this type is essentially a “cancer” of pulmonary arteriolar endothelial cells (see id.), at least because in the initial or early stages of the disease, an acute apoptotic loss of normal endothelial cells may result in the emergence and clonal expansion of apoptosis resistant endothelial cells. Lee et al. (1998). The neoplastic process associated with PAH provides for not only kinase inhibitor treatment of PAH, but also the development of new compounds, compositions, and methods, via MCT+PN model determinations, with superior efficacy and potency compared to previously generated kinase inhibitors using inferior model systems, for the treatment of neoplastic disease. Drug-kinase homology modeling ensures that such inhibitors, including, for example, irreversibly derivatives thereof, target vulnerable kinase domains for optimal efficacy.

In one aspect, the present disclosure provides compounds of Structure 1, tautomers of the compounds, pharmaceutically acceptable salts of the compounds, pharmaceutically acceptable salts of the tautomers, and mixtures thereof, wherein Structure 1 is shown below.

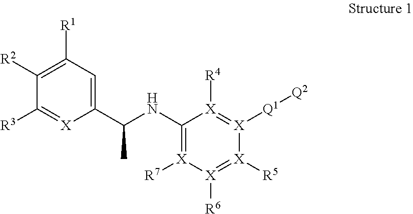

Where X, R¹, R², R³, R⁴, R⁵, R⁶, R⁷ (and R⁸, R⁹, R¹⁰, R¹¹, and R¹², as contained therein), Q¹ and Q² (and Q³ as contained therein) are selected from “XRQ”, as noted in the Summary.

The structure of R⁸ has the following formula in suitable embodiments:

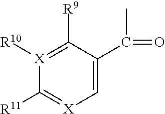

where X, R⁹, R¹⁰, R¹¹ and R¹² are selected from “XRQ2”, as noted in the summary.

In some embodiments, the structure of Q¹ and/or Q² is selected from the group consisting of —C—N—C(═O)—C═C, —C—N—C(═O)—C—F,

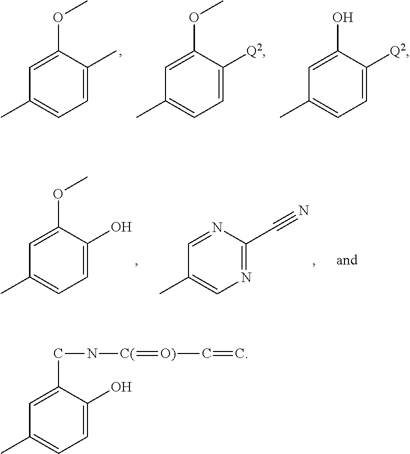

The compound of Structure I is a compound of Structure II (or 2) in some embodiments, where the compound of Structure 2, i.e., (S)—N-(3-(1-((6-(4-hydroxy-3-methoxyphenyl)pyrazin-2yl)amino)ethyl)phenyl)-5-methylnicotinamide, referred to herein as “PK10453” is as shown in the Summary.

In some embodiments, the compound of Structure 1 is a compound of Structure 3, 4, 5, 6, 7, 8, 9, 10, 11, or 12, as illustrated in Chart A above, where R is H, F, CH₃, or CF₃.

In some embodiments, the compound of Structure 1 is an irreversible kinase inhibitor, as further detailed below, where the Structure 1 compound is a compounds of Structure 13, 14, 15, 16, 17, 18, 19, 20, 21, 22, 23, 24, 25, 26, 27, 28, 29, 30, 31, 32, 33 or 34 as shown in Chart B of the Summary above.

### Compound Synthesis

In one aspect, the present disclosure provides for the synthesis of Structure I compounds, which are readily synthesized using the procedures described in the following sections and as disclosed in WO 2008/058341, which is hereby incorporated by reference in its entirety and for all purposes as if fully set forth herein.

Compounds of Structure I are typically prepared from starting materials, such as, e.g., dihaloheterocycle. The first step is a nucleophilic aromatic substitution to generate a monoamino-monohalo intermediate. The nucleophilic aromatic substitution is typically carried out by addition of a primary or secondary amine to the di-halogenated heterocycle in a solvent such as ethanol, isopropanol, tert-butanol, dioxane, THF, DMF, ethoxyethanol, toluene or xylene. The reaction is typically performed at elevated temperature in the presence of excess amine or a non-nucleophilic base such as triethylamine or diisopropylethylamine, or an inorganic base such as potassium carbonate or sodium carbonate.

Alternatively, the amino substituent may be introduced through a transition metal catalyzed amination reaction. Typical catalysts for such transformations include Pd(OAc)₂/P(t-Bu)₃, Pd₂(dba)₃/BINAP and Pd(OAc)₂/BINAP. These reactions are typically carried out in solvents such as toluene or dioxane, in the presence of bases such as caesium carbonate or sodium or potassium tert-butoxide at temperatures ranging from room temperature to reflux. See, e.g., Hartwig and Angew, Chem. Int. Ed 37, 2046 (1998). The amines employed in the first step of the synthesis of these compounds are obtained commercially or are prepared using methods well known to those skilled in the art. α-alkylbenzylamines, moreover, may be prepared through reduction of oximes. Typical reductants include lithium aluminium hydride, hydrogen gas in the presence of palladium on charcoal catalyst, Zn in the presence of hydrochloric acid, sodium borohydride in the presence of a Lewis acid such as TiCb, ZrCU, NiCl₂ and MoO₃, or sodium borohydride in conjunction with Amberlyst H1 5 ion exchange resin and LiCl. α-Alkylbenzylamines may also be prepared by reductive amination of the corresponding ketones. A classical method for such a transformation is the Leuckart-Wallach reaction, though catalytic conditions (HCO₂NH₄, [(CH₃)₅C₅RhCl₂]₂) or alternative procedures, e.g., NH₄OAc, Na(CN)BH₃) can also be used. α-Alkylbenzylamines may also be prepared from the corresponding α-alkylbenzyl alcohols. Such methods include derivatisation of the hydroxyl as a mesylate or tosylate and displacement with a nitrogen nucleophile, such as phthalimide or azide which is converted to the primary amine using conventional synthetic methods; or, displacement of the hydroxyl with a suitable nitrogen nucleophile under Mitsunobu-like conditions. α-Alkylbenzyl alcohols can be prepared by reduction of the corresponding ketones with a reducing agent such as sodium borohydride in a solvent such as methanol. Alternatively, α-alkylbenzyl alcohols can be obtained through addition of an alkyl metal species (such as a Grignard reagent) to a benzaldehyde derivative, typically performed at room temperature or below in solvents such as tetrahydrofuran. α-Alkyl benzylamines of high optical purity may be prepared from chiral α-alkyl benzyl alcohols using the methods outlined above. The chiral α-alkyl benzyl alcohols may be obtained through chiral reduction of the corresponding ketones. Chiral reducing methods are now well known in organic chemistry and include enzymatic processes, asymmetric hydrogenation procedures and chiral oxazaborolidines.

The monoamino-monohalo intermediate formed from the dihaloheterocycle and the amine described above, may then be further functionalized. For example, where the amine substituent bears an additional functional group, this functional group may be derivatized or functionalized using methods well-known to those skilled in the art. For example, a free primary amino group could be further functionalized to an amide, sulphonamide or urea functionality, or could be alkylated to generate a secondary or tertiary amine derivative. Preferable methods for the formation of an amide include coupling the amine with a carboxylic acid using coupling reagents such as dicyclohexylcarbodiimide, 1-(3-dimethylaminopropyl)-3-ethylcarbodiimide, diisopropylcarbodiimide or carbonyldiimidazole in solvents such as dichloromethane, tetrahydrofuran or 1,4-dioxane. Alternatively, the acid component may be activated by conversion to an acid chloride (using thionyl chloride, oxalyl chloride, bis(trichloromethyl)carbonate or cyanuric chloride) or to mixed anhydride species (using, for example, t-butyl chloroformate or isopropyl chloroformate) or to active ester intermediates (such as N-hydroxysuccinimidyl, pentafluorophenyl or p-nitrophenyl esters) prior to reaction with the amine.

The monoamino-monochloro intermediate may then be reacted in a palladium mediated cross-coupling reaction with a suitably functionalized coupling partner to replace the halogen atom with an alternative moiety. Typical coupling partners are organoboronic acids or esters. See, e.g., Miyaura and Suzuki, Chem Rev. 952457 (1995); Stille, Chem., Int. Ed. Engl 25, 508 (1986); Kumada et al., Org. Synth. Coll. Vol. 6, 407 (1998); and: Negishi, J. Organomet. Chem. 653, 34 (2002) for Suzuki coupling, organostannanes, Stille coupling, Grignard reagents, Kumada coupling, organozinc species, and Negishi coupling, respectively.

The Suzuki coupling is the preferred coupling method and is typically performed in a solvent such as DME, THF, DMF, ethanol, propanol, toluene, or 1,4-dioxane in the presence of a base such as potassium carbonate, lithium hydroxide, caesium carbonate, sodium hydroxide, potassium fluoride or potassium phosphate. The reaction may be carried out at elevated temperatures and the palladium catalyst employed may be selected from Pd(PPh₃)₄, Pd(OAc)₂, [PdCl₂(dppf)], Pd₂(dba)₃/P(t-Bu)₃.

The monoamino-monochloro intermediate may also be subjected to a second nucleophilic aromatic substitution reaction using similar conditions to those outlined above. Those skilled in the art will appreciate that the order of the reactions described for the syntheses above may be changed in certain circumstances and that certain functionalities may need to be derivatized, i.e., protected, in certain instances for the reactions described above to proceed with reasonable yield and efficiency. The types of protecting functionality are well-known to those skilled in the art. The products formed from the reaction sequences described above may be further derivatized using techniques well known to those skilled in the art.

The leaving group may be any suitable known type such as those disclosed in March, “Advanced Organic Chemistry: Reactions, Mechanisms and Structure.” 4th Ed. pp 352-7, Wiley & Sons, NY (1992), which is incorporated herein by reference. In some embodiments, the leaving group is a halogen, e.g., chlorine.

Irreversible compounds, i.e., kinase inhibitors that are capable of covalently binding to one or more target kinases, were synthesized in accord with the procedures above, while further linking a covalent moiety, as described in the following documents. See, e.g., US 2008/0268460; Leproult et al., J. Med. Chem. 54, 1347-1355 (2011); Discafani et al., Biochem. Biopharmacol. 57:917-925 (1999); Frey et al., Proc. Natl. Acad. Sci. U.S.A. 95:12022-12027 (1998); Barf et al., J. Med. Chem. 2012, 55, 6243-6262 (2012).

### Kinases

Protein kinases are a family of enzymes that catalyze the phosphorylation of specific residues in proteins. Such enzymes are generally categorized into three groups, those which preferentially phosphorylate serine and/or threonine residues, those which preferentially phosphorylate tyrosine residues, and those which phosphorylate both tyrosine and Ser/Thr residues. Protein kinases are therefore key elements in signal transduction pathways responsible for transducing extracellular signals, including the action of cytokines on their receptors, to the nuclei, triggering various biological events. The many roles of protein kinases in normal cell physiology include cell cycle control including proliferation and cell growth, differentiation, metabolism, apoptosis, cell mobility, mitogenesis, transcription, translation and other signaling processes. Descriptions of protein kinases are known in the art and are detailed in U.S. Provisional Patent Application No. 61/751,217.

### Kinase Inhibitors

In one aspect, the present disclosure provides compounds and methods of inhibiting a kinase, e.g., a tyrosine kinase, such as a RTK, in a subject and/or a method of treating a biological condition mediated by, or associated with, a kinase, e.g., a tyrosine kinase, such as a RTK, in a subject. In some embodiments, the kinase is Cdc2 kinase, c-Kit, c-ABL, p60src, VEGFR3, PDGFRα, PDGFRβ, FGFR3, PDGFR-αα, PDGFR-ββ, PDGFR-αβ, FLT-3, Fyn, Lck, Tie-2, GSK-3, Cdk2, Cdk4, MEK1, NEK-2, CHK2, CK1ε, Raf, CHK1, Rsk2, FMS (CSF-IR), KDR, EphA2, EphA3, EphA8, FLT1, FLT4, HCK, PTK5, RET, SYK, DDR1, DDR2 and PAR-1. Likewise, the kinase is a tyrosine kinase, such as, e.g., Cdc2 kinase, c-Kit, c-ABL, p60src, VEGFR3, PDGFRα, PDGFRβ, FGFR3, PDGFR-αα, PDGFR-ββ, PDGFR-αβ, FLT-3, Fyn, Lck, and/or Tie-2, in some embodiments. The methods include administering to the subject a compound of Structure I, a tautomer of the compound, a pharmaceutically acceptable salt of the compound, a pharmaceutically acceptable salt of the tautomer, or mixtures thereof. In the method of inhibiting a tyrosine kinase, the tyrosine kinase is inhibited in the subject after administration.

Previously, various indolyl substituted compounds are shown to inhibit one or more kinases, as disclosed in WO 01/29025, WO 01/62251, and WO 01/62252. Likewise, various benzimidazolyl compounds have recently been disclosed in WO 01/28993. Such compounds are reported to be capable of inhibiting, modulating, and/or regulating signal transduction of both receptor-type and non-receptor tyrosine kinases. Some of the disclosed compounds contain a quinolone fragment bonded to the indolyl or benzimidazolyl group.

The synthesis of 4-hydroxy quinolone and 4-hydroxy quinoline derivatives has also been reported. For example, Ukrainets et al. have disclosed the synthesis of 3-(benzimidazol-2-yl)-4-hydroxy-2-oxo-1,2-dihydroquinoline. Ukrainets, I. et al., Tet. Lett. 42, 7747-7748 (1995); Ukrainets, I. et al., Khimiya Geterotsiklicheskikh Soedinii, 2, 239-241(1992). Ukrainets has also disclosed the synthesis, anticonvulsive and antithyroid activity of other 4-hydroxy quinolones and thio analogs such as 1H-2-oxo-3-(2-benzimidazolyl)-4-hydoxyquinoline. Ukrainets, I. et al., Khimiya Geterotsiklicheskikh Soedinii, 1, 105-108 (1993); Ukrainets, I. et al., Khimiya Geterotsiklicheskikh Soedinii, 8, 1105-1108 (1993); Ukrainets, I. et al., Chem. Heterocyclic Comp. 33, 600-604, (1997).

Other compounds, such as, for example, 4-Amino-5-fluoro-3-[5-(4-methylpiperazin-1-yl)-1H-benzimidazol-2-yl]quinolin-2(1H)-one has been described as an orally bioavailable benzimidazole-quinolinone that exhibits inhibition of receptor tyrosine kinases that drive both endothelial and tumor cell proliferation. The inhibitory effect of this compound was shown on nine tyrosine kinases, FGFR1, FGFR3, VEGFR1, VEGFR2, VEGFR3, PDGFRβ, c-Kit, p60src, and FLT-3, as disclosed in WO 2005/047244. However, this compound does not significantly inhibit EGFR kinases or insulin receptor kinases at clinically acceptable doses.

Moreover, 4-(4-methylpiperazin-1-ylmethyl)-N-[4-methyl-3-(4-pyridin-3-yl)pyrimidin-2-ylamino)phenyl]-benzamide (imatinib), as disclosed in US 2006/0154936, inhibits PDGFRα and β kinases, Abl, DDR, and c-KIT, as described in US 2011/0190313. Paragraph [0117] of US 2011/0190313, however, indicates that although imatinib appeared safe and well tolerated over a 6 month period, the primary efficacy parameter (6MWD) did not improve in patients randomized to imatinib compared with placebo, despite significant improvement in secondary endpoints. A continuing need therefore exists for compounds that inhibit, kinases, e.g., tyrosine kinases, such as RTKs, at least because of previous limitations, resistant disease phenotypes, and the need for more effective kinase, e.g., RTK, inhibition, as further detailed below. See, e.g., US 2008/0268460.

While most RTK inhibitors are believed to reversibly bind a target receptor, compounds that irreversibly bind to certain target receptors have been shown to be superior tumor suppressors. See Frey et al., Proc. Natl. Acad. Sci. U.S.A. 95:12022-12027 (1998). These compounds impart potent suppressive effects with respect to a variety of disease indications in certain animal models. Others have reported that irreversible EGFR kinase inhibitors effectively suppress growth in human tumor cell models. See Discafani et al., Biochem. Biopharmacol. 57:917-925 (1999). Hence, compounds that irreversibly bind kinases, tyrosine kinases, and/or RTKs provide therapeutic indications possessing superior inhibitory effects compared to reversible kinase inhibitors. See, e.g., U.S. 2008/0268460.

To this end, the advantages of irreversible RTK inhibitors are many. First, such inhibitors would not compete with ATP. Tyrosine kinases typically catalyze the transfer of a phosphate group from a molecule of ATP to a tyrosine residue located on a protein substrate. Since the concentration of ATP in a cell is normally very high (mM), compounds that are competitive with ATP may show diminished efficacy and duration of action since it would be difficult for such compounds to reach the concentrations within the cell that are necessary to displace the ATP from its binding site for an extended time. Compounds which covalently bind to and inhibit tyrosine kinases would be non-competitive with ATP or protein substrates. Indeed, because prolonged kinase suppression is most likely necessary for maximum efficacy, an irreversibly bound inhibitor provides an advantage by permanently eliminating the existing kinase activity, which should return only when a new receptor is synthesized.

Lower plasma levels of the inhibitor is still a further advantage. The irreversible binding inhibitors require that plasma concentrations be attained only long enough to expose the inhibitor to the target. After the irreversible inhibitor binds, no more inhibitor is needed in the plasma in order to maintain inhibition. Thus, there is less likelihood of toxicity, which results from high or prolonged plasma levels. Covalent inhibitor interaction with RTKs, furthermore, likely possess less cross-reactivity with other kinases containing homologous amino acids in their active site, e.g., platelet-derived growth factor receptor (PDGFR) and vascular endothelial growth factor receptor 1 (VEGFR-1).

Furthermore, the small molecules that were reported in Frey et al. (1998) were shown to irreversibly inhibit epidermal growth factor receptor (EGFR) by covalently interacting with the receptor, while alkylating a cysteine residue in the ATP binding pocket of the molecule. Indeed, Leproult et al., J. Med. Chem. 54, 1347-1355 (2011), discloses that one approach to designing irreversible inhibitors is to exploit the nucleophilicity of a cysteine thiol group present in the target protein via systematic analysis of cysteine residues present in the nucleotide binding site of kinases. Such an approach can facilitate irreversible inhibition even when considering various kinase conformations and thus improve dosing and toxicity.

The cysteine mapping in Leproult et al. (2011) demonstrate that kinases are potential targets for selective covalent inhibitors. An example is shown of the kinase inhibitor imatinib to which a chloroacetamide group is added in the para position of the benzene ring. Peptide inhibitor adduct formation was shown for both Kit and PDGFα receptors. Id. However, other compounds failed to show similar covalent adducts. Chloroacetamide is shown as an example of an electrophile which can form a covalent bond with a cysteine residue. The general term “warhead” is used to mean an electrophilic trap for forming a covalent bond between the inhibitor and the targeted protein kinase. Chloroacetamide as an electrophile may be too reactive to have clinical utility and may have toxicity for this reason. Leproult et al. (2011) nevertheless suggest that less then optimal positioning of the electrophile may explain why a covalent bond may not form with less reactive warheads.

The present disclosure provides for distinct warhead positioning on RTK receptor inhibitors. In some embodiments, electrophiles other than those described by Leproult et al. (2011), were employed for increased efficacy. See Barf et al. (2012) and Oballa et al., Bioorg Med Chem Lett 17:998-1002 (2007) (describing nitrile-containing electrophiles). Further, Diller et al., J Med Chem 46:4638-4647 (2003) reported a homology model of the PDGFβ receptor based on VEGFR2 (55% homology). The PDB files, however, are not available.

Molecular docking was employed with respect to one aspect of the present invention by using homology models of RTK, based on homologous structures, e.g., PDGFα and PDGFβ receptor homology to c-Kit is 59% and 63%, respectively. See Examples. In some embodiments, the introduction of various electrophiles in a variety of positions with respect to a RTK inhibitor, e.g., PDGFR inhibitor, scaffold provided the bases for further biochemical analyses. To this end, the spatial orientation of the inhibitor warheads, relative to the target cysteine residues, can be analyzed to calculate the free energy of binding and estimated Ki. In some embodiments, compounds with the lowest free energy of binding and closest proximity (of warhead to a CYS residue) impart irreversible selective RTK inhibitors.

Accordingly, the present disclosure provides compounds of Structure 1, the tautomer of the compound, the pharmaceutically acceptable salt of the compound, the pharmaceutically acceptable salt of the tautomer, or the mixture thereof, which covalently interact with a receptor tyrosine kinase (RTK), such as, for example, PDGFR or c-Kit or both. In some embodiments, the PDGFR is selected from PDGFR-α, PDGFR-β, PDGFR-αα, PDGFR-ββ, and PDGFR-αβ as demonstrated by homology modeling.

The homology modeling described above can be performed using NCBI BLAST search techniques to examine the available templates. RTKs that are found to possess the highest identity to the query sequences are selected. Template crystal structures can be found in the Brookhaven Protein DataBank, and initial sequence alignments of the template and query sequences are subjected to ClustalW program analysis. See Thompson et al., Nucleic Acids Res. 22 (22): 4673-80 (1994). Three-dimensional (3D) models comprising all non-hydrogen atoms can be generated using MODELLER9v 11. See Sali et al. (1993).

Models from random generation of the starting structure is calculated in some embodiments. Each generated model is first optimized with, for example, the variable target function method (VTFM) with conjugate gradients (CG), and subsequently further refined utilizing molecular dynamics (MD) with simulated annealing (SA). Subsequently, the best DOPE score model is selected as final template. The quality of the model was evaluated using PROCHECK (see Laskowski et al., Journal Of Applied Crystallography 26:283-91 (1993)), and where 100% of the residues found in allowable regions of a Ramachandran diagram. See Examples. Irreversible RTK inhibitors are then modeled in accordance with the homology model template of, e.g., PDGF, PDGFR, etc.

Such compounds are modeled to possess covalent interactions with one or more RTKs depending of the template employed. In some embodiments, the compounds of the present disclosure were shown, via modeling interactions, to interact with one or more of the PDGFR, PDGFRα, PDGFRβ, PDGFR-αα, the PDGFR-ββ, and/or the PDGFR-αβ amino acids selected from the group consisting of LYS627, VAL607, GLU644, MET648, HIS816, LEU809, ASP836, CYS814, ILE834, CYS835, PHE937, LYS634, VAL614, GLU651, MET655, HIS824, LEU817, ASP844, CYS822, ILE842, VAL658, ILE647, HIS816, ARG836, LYS634, GLU651, ALA632, HIS824, MET655, ARG825, CYS843, THR874, VAL607, ARG817, VAL815, LEU651, LEU809, ILE657, THR681, ILE654, ARG825, ASP826, LEU658, LEU825, PHE837, LEU658, HIS824, CYS814, ILE654, ASP844, ILE842, and CYS843. See Table 7 below.

Covalent kinase inhibitors of the present disclosure, include, but are not limited to compounds of Structure 1, tautomers of the compound, pharmaceutically acceptable salts of the compound, pharmaceutically acceptable salts of the tautomer, or mixtures thereof, where Structure 1 has the following formula:

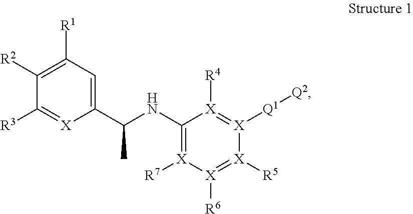

Where X, R¹, R², R³, R⁴, R⁵, R⁶, R⁷ (and R⁸, R⁹, R¹⁰, R¹¹, and R¹², as contained therein), Q¹ and Q² (and Q³ as contained therein) are selected from “XRQ”, as noted in the Summary, and where the elevated pulmonary pressure in the subject is elevated compared to a healthy subject in a control population, and further wherein the reduced pulmonary pressure is reduced compared to the pulmonary pressure in the subject prior to the administering.

In some embodiments, the structure of R⁸ has the following formula:

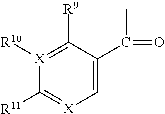

where X, R⁹, R¹⁰, R¹¹ and R¹² are selected from “XRQ2”, as noted above in the Summary.

In some embodiments, the structure of Q¹ and/or Q² is selected from the group consisting of —C—N—C(═O)—C═C, —C—N—C(═O)—C—F,

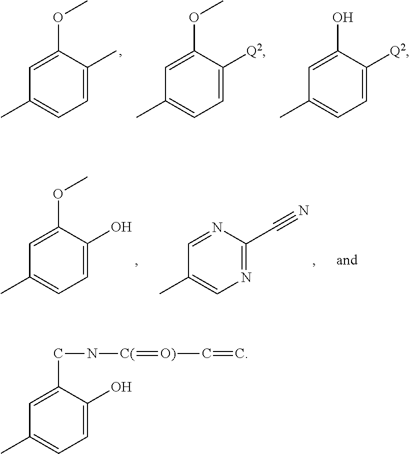

In suitable embodiments, the compound of Structure 1 is a compound of Structure 2 as identified in the Summary.

In some embodiments, the irreversible inhibitor is selected from a compound of Structure 3, 4, 5, 6, 7, 8, 9, 10, 11, and 12, as noted in Chart A, where R is H, F, CH₃, or CF₃.

In some embodiments, the irreversible inhibitor is selected from the group consisting of a compound of Structure 13, 14, 15, 16, 17, 18, 19, 20, 21, 22, 23, 24, 25, 26, 27, 28, 29, 30, 31, 32, 33, and 34 as noted above in Chart B.

### Pharmaceutical Compositions

In one aspect, the present disclosure provides pharmaceutical compositions which include at least one of the compounds of Structure 1 and a pharmaceutically acceptable carrier. The compositions of the present invention may contain other therapeutic agents as described below, and may be formulated, for example, by employing conventional solid or liquid vehicles or diluents, as well as pharmaceutical additives of a type appropriate to the mode of desired administration, for example, excipients, binders, preservatives, stabilizers, flavors, etc., according to techniques such as those well known in the art of pharmaceutical formulation. See, e.g., Remington: The Science and Practice of Pharmacy, 21st Ed., Lippincott Williams & Wilkins (2005). Descriptions of pharmaceutical compositions are, in part, known in the art and are further detailed in U.S. Provisional Patent Application No. 61/751,217, which is hereby incorporated by reference in its entirety.

In short, pharmaceutical compositions are typically formulated to be compatible with its intended route of administration. Examples of routes of administration include parenteral (e.g., intravenous, intradermal, intraperitoneal or subcutaneous), oral, inhalation, transdermal (topical), intraocular, iontophoretic, and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. For convenience of the patient or treating physician, the dosing formulation can be provided in a kit containing all necessary equipment (e.g., vials of drug, vials of diluent, syringes and needles) for a treatment course.

The compounds of the present disclosure are administered by any suitable means, for example, orally, such as in the form of tablets, capsules, granules or powders; sublingually; buccally; parenterally, such as by subcutaneous, intravenous, intramuscular, intra(trans)dermal, or intracisternal injection or infusion techniques, e.g., as sterile injectable aqueous or non-aqueous solutions or suspensions, nasally such as by inhalation spray or insufflation, topically, such as in the form of a cream or ointment ocularly in the form of a solution or suspension. The compounds may, for example, be administered in a form suitable for immediate release or extended release. Immediate release or extended release may be achieved by the use of suitable pharmaceutical compositions comprising the present compounds, or, particularly in the case of extended release, by the use of devices such as subcutaneous implants or osmotic pumps.

The pharmaceutical compositions for the administration of the compounds of Structure 1 are presented in unit dosage form and may be prepared by any of the methods well known in the art of pharmacy, in some embodiments. The pharmaceutical compositions containing the compound of Structure 1, in some embodiments, are in a form suitable for oral use, for example, as tablets, troches, lozenges, aqueous or oily suspensions, dispersible powders or granules, emulsions, hard or soft capsules, or syrups or elixirs. Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols.

For administration to the respiratory tract, e.g., inhalation, including intranasal administration, the active compound may be administered by any of the methods and formulations employed in the art for administration to the respiratory tract. Thus, the active compound may be administered in the form of a solution, suspension, or as a dry powder, in some embodiments. The agents according to this aspect of the present invention may also be administered directly to the airways in the form of an aerosol. For use as aerosols, the compounds of the present invention in solution or suspension may be packaged in a pressurized aerosol container together with suitable propellants, for example, hydrocarbon propellants like propane, butane, or isobutane with conventional adjuvants. Such materials also may be administered in a non-pressurized form such as in a nebulizer or atomizer.

The propellant-driven inhalation aerosols which may be used according to the invention may also contain other ingredients such as co-solvents, stabilizers, surfactants, antioxidants, lubricants and pH adjusters. The propellant-driven inhalation aerosols according to the invention which may be used according to the invention may be administered using inhalers known in the art, e.g., metered dose inhalers. As another alternative, the agents of the present invention may be administered to the airways in the form of a lung surfactant formulation. The lung surfactant formulation can include exogenous lung surfactant formulations (e.g., Infasurft (Forest Laboratories), Survanta® (Ross Products), and Curosurf® (DEY, California, USA) or synthetic lung surfactant formulations (e.g., Exosurft (GlaxoWellcome Inc.) and ALEC). These surfactant formulations are typically administered via airway instillation (i.e., after intubation) or intratracheally.

As a further alternative, the agents of the present invention may be administered to the airways in the form of an inhalable powder. The powder formulation may include physiologically acceptable excipients such as monosaccharides (e.g. glucose or arabinose), disaccharides (e.g. lactose, saccharose, maltose, trehalose), oligo- and polysaccharides (e.g. dextrane), polyalcohols (e.g. sorbitol, mannitol, xylitol), salts (e.g. sodium chloride, calcium carbonate) or mixtures of these excipients with one another. Preferably, mono- or disaccharides are used, while the use of lactose or glucose is preferred (but not exclusively) in hydrate form.

Within the scope of the inhalable powders according to the invention the excipients have a maximum average particle size of up to 250 μm, preferably between 10 and 150 μm, most preferably between 15 and 80 μm. It may sometimes seem appropriate to add finer excipient fractions with an average particle size of 1 to 9 μm to the excipients mentioned above. These finer excipients are also selected from the group of possible excipients listed hereinbefore. Finally, in order to prepare the inhalable powders according to the invention, micronised formulations, preferably with an average particle size of 0.5 to 10 μm is added to the excipient mixture. Processes for producing the inhalable powders according to the invention by grinding and micronizing and by finally mixing the ingredients together are known from the prior art. Inhalable powders according to the invention which contain a physiologically acceptable excipient in addition to the active formulation may be administered, for example, by means of inhalers which deliver a single dose from a supply using a measuring chamber as described in U.S. Pat. No. 4,570,630, or by other means as described in DE 36 25 685A, each of which are incorporated by reference in its entirety.

As a still further alternative, the agents of the present invention may be administered to the airways in the form of a propellant-free inhalable solution and suspension. The solvent used may be an aqueous or alcoholic, preferably an ethanolic solution. The solvent may be water on its own or a mixture of water and ethanol. The relative proportion of ethanol compared with water is not limited but the maximum is up to 70 percent by volume, more particularly up to 60 percent by volume and most preferably up to 30 percent by volume. The remainder of the volume is made up of water. The solutions or suspensions containing the active formulation are adjusted to a pH of 2 to 7, preferably 2 to 5, using suitable acids.

In formulations intended for administration to the respiratory tract, including intranasal formulations, the active compound is typically configured to have a small particle size, e.g., approximately 5 microns or less, via micronisation techniques and the like. Sustained release formulations of the active compound are employed in some embodiments, while, in others, it is administered by oral inhalation as a free-flow powder (e.g., via inhaler).

The pharmaceutical composition and method of the present disclosure further include additional therapeutically active compounds (second agents), as noted herein and/or known in the art, which are typically employed for treating one or more pathological conditions in concert with the compositions comprising compounds of Structure 1 of the present disclosure. The combination of therapeutic agents acts synergistically to effect the treatment or prevention of the various diseases, disorders, and/or conditions described herein. Such second agents, include, but are not limited to, of prostanoids, endothelin antagonists, cytoplasmic kinase inhibitors, receptor kinase inhibitors, endothelin receptor antagonists, e.g., ambrisentan, bosentan, and sitaxsentan, PDE5 (PDE-V) inhibitors, e.g., sildenafil, tadalafil, and vardenafil, calcium channel blockers, e.g., amlodipine, felodipine, varepamil, diltiazem, and menthol, prostacyclin, treprostinil, iloprost, beraprost, nitric oxide, oxygen, heparin, warfarin, diuretics, digoxin, cyclosporins, e.g., cyclosporin A, CTLA4-Ig, antibodies such as ICAM-3, anti-IL-2 receptor (Anti-Tac), anti-CD45RB, anti-CD2, anti-CD3 (OKT-3), anti-CD4, anti-CD80, anti-CD86, agents blocking the interaction between CD40 and gp39, such as antibodies specific for CD40 and/or gp39, i.e., CD 154, fusion proteins constructed from CD40 and gp39 (CD401g and CD8gp39), inhibitors, such as nuclear translocation inhibitors, of NF-kappa B function, such as deoxyspergualin (DSG), cholesterol biosynthesis inhibitors such as HMG CoA reductase inhibitors (lovastatin and simvastatin), non-steroidal anti-inflammatory drugs (NSAIDs) such as ibuprofen, aspirin, acetaminophen, leflunomide, deoxyspergualin, cyclooxygenase inhibitors such as celecoxib, steroids such as prednisolone or dexamethasone, gold compounds, beta-agonists such as salbutamol, LABAs such as salmeterol, leukotriene antagonists such as montelukast, antiproliferative agents such as methotrexate, FK506 (tacrolimus, Prograf), mycophenolate mofetil, cytotoxic drugs such as azathioprine, VP-16, etoposide, fludarabine, doxorubin, adriamycin, amsacrine, camptothecin, cytarabine, gemcitabine, fluorodeoxyuridine, melphalan and cyclophosphamide, antimetabolites such as methotrexate, topoisomerase inhibitors such as camptothecin, DNA alkylators such as cisplatin, kinase inhibitors, e.g., sorafenib, microtubule poisons, e.g., paclitaxel, TNF-α inhibitors, e.g., tenidap, α-TNF antibodies or soluble TNF receptor, hydroxy urea and rapamycin (sirolimus or Rapamune) or derivatives.

The compounds of the invention may also be prepared as salts which are pharmaceutically acceptable, but it will be appreciated that non-pharmaceutically acceptable salts also fall within the scope of the present disclosure at least to the extent that such salts are useful as intermediates in the preparation of pharmaceutically acceptable salts. Examples of pharmaceutically acceptable salts are generally known in the art.

Where a compound possesses a chiral center the compound can be used as a purified enantiomer or diastereomer, or as a mixture of any ratio of stereoisomers. It is however preferred that the mixture comprises at least 1, 5, 10, 20, 30, 40, 50, 60, 70, 80, 90, 95, 97.5 or 99% of the preferred isomer. The compound may also exist as tautomers. In some embodiments, the mixture comprises at least 70, 90, 95 or 99% of the isomer.

In some embodiments, the compounds of the present disclosure are administered in a therapeutically effective amount. Such an administration imparts that a compound of Structure 1 will elicit a response associated with, e.g., cells, tissues, fluids, of a subject being sought by the clinician. In the treatment or prevention of conditions mediated by, or associated with, kinase inhibition, e.g., RTK inhibition, an appropriate dosage level is administered. In some embodiments, from about 0.01 to 500 mg/kg of subject body weight per day is administered in single or multiple doses. In accord, dosage levels are from about 0.1 to about 250 mg/kg per day in some embodiments, while in other embodiments from about 0.5 to about 100 mg/kg per day is administered to the subject. Suitable dosage levels include, for example, from about 0.01 to 250 mg/kg per day, from about 0.05 to 100 mg/kg per day, or from about 0.1 to 50 mg/kg per day. Within this range, in some embodiments, the dosage is from about 0.05 to 0.5, 0.5 to 5 or 5 to 50 mg/kg per day. For oral administration, the compositions are provided in the form of tablets containing 1.0 to 1000 mg of the active ingredient, including, but not limited to, 1, 5, 10, 15, 20, 25, 50, 75, 100, 150, 200, 250, 300, 400, 500, 600, 750, 800, 900, and 1000 mg of the active ingredient. The dosage may be selected, for example, to any dose within any of these ranges, for therapeutic efficacy and/or symptomatic adjustment of the dosage to the subject being treated. In some embodiments, the compounds of the present disclosure are administered by inhalation as described in, e.g., U.S. Pat. No. 8,257,741, U.S. Pat. No. 8,263,128, WO 2010/132827, WO 2010/102066, WO 2012/040502, WO 2012/031129, and/or WO 2010/102065, from 1 to 20, 1 to 15, 1 to 10, 1 to 5, 1 to 4, or 1 to 3 times daily, or once or twice/d. In some embodiments, the compounds are administered 1-5/d.

In some embodiments, the unit dose is sufficient to provide one or more of: (a) a Cₘₐₓ of about 1 to 5000 ng/mL of the compound In a subject's plasma or a Cₘₐₓ of about 1 to 5000 ng/mL of the compound In the subject's blood when it is administered to the subject; and (b) about 1 to 5000 ng/mL of the compound in a subject's plasma 24 h after administration or about 1 to 5000 ng/mL of the compound in the subject's blood 24 h after administration to the subject.

The therapeutically effective amount of a compound of Structure 1, the tautomer of the compound, the pharmaceutically acceptable salt of the compound, the pharmaceutically acceptable salt of the tautomer, or the mixture thereof, is not associated with adverse side effects, in some embodiments. Such adverse side effects include, but are not limited to, decreased lung function, altered systemic BP, immunocompromised, bone marrow suppression, anemia, hypoxia, in the subject compared to prior to the administering.

### Prevention and Treatment of Disease

In one aspect, the present disclosure provides a compound of Structure 1, a tautomer of the compound, a pharmaceutically acceptable salt of the compound, a pharmaceutically acceptable salt of the tautomer, or a mixture thereof, for treating one or more diseases, wherein a compound of Structure 1 is described herein. See, e.g., Summary.

The present disclosure accordingly provides compounds, compositions, and methods of inhibiting kinases, e.g., tyrosine kinases, and methods of treating biological conditions mediated by, or associated with, such kinases. For example, the present disclosure provides methods of inhibiting one or more kinases, such as, e.g., cell division cycle 2 kinase (Cdc2 kinase), c-Kit, c-ABL, p60src, VEGFR3, PDGFRα, PDGFRβ, PDGFR-αα, PDGFR-ββ, PDGFR-αβ, FGFR3, FLT-3, FYN oncogene kinase related to SRC, FGR, YES (Fyn), lymphocyte-specific protein tyrosine kinase (Lck), tyrosine kinase with Ig and EGF homology domains (Tie-2), FMS (CSF-IR), KDR, EphA2, EphA3, EphA8, FLT1, FLT4, HCK, PTK5, RET, SYK, DDR1, DDR2, glycogen synthase kinase 3 (GSK-3), cyclin dependent kinase 2 (Cdk2), cyclin dependent kinase 4 (Cdk4), MEK1, NEK-2, CHK2, CK1, Raf, checkpoint kinase 1 (CHK1), ribosomal S6 kinase 2 (Rsk2), and PAR-1. In particular, the present disclosure provides for compounds, compositions, and methods of inhibiting tyrosine kinases, such as, e.g., cell division cycle 2 kinase (Cdc2 kinase), c-Kit, c-ABL, p60src, VEGFR3, PDGFRα, PDGFRβ, PDGFR-αα, PDGFR-ββ, PDGFR-αβ, FGFR3, FLT-3, FYN oncogene kinase related to SRC, FGR, YES (Fyn), lymphocyte-specific protein tyrosine kinase (Lck), tyrosine kinase with Ig and EGF homology domains (Tie-2), FMS (CSF-IR), KDR, EphA2, EphA3, EphA8, FLT1, FLT4, HCK, PTK5, RET, SYK, DDR1, and DDR2. In some embodiments, the tyrosine kinase is a receptor tyrosine kinase (RTK), such as, e.g., PDGFR, PDGFR-αα, PDGFR-ββ, PDGFR-αβ, or c-Kit, or combinations thereof.

The present disclosure also provides compounds, compositions, and methods of treating biological conditions mediated by, or associated with, kinases, e.g., tyrosine kinases, including Cdc2 kinase, c-Kit, c-ABL, p60src, VEGFR3, PDGFRα, PDGFRβ, FGFR3, PDGFR-αα, PDGFR-ββ, PDGFR-αβ, FLT-3, Fyn, Lck, Tie-2, GSK-3, Cdk2, Cdk4, MEK1, NEK-2, CHK2, CK1ε, Raf, CHK1, Rsk2, FMS (CSF-IR), KDR, EphA2, EphA3, EphA8, FLT1, FLT4, HCK, PTK5, RET, SYK, DDR1, DDR2 and PAR-1. In particular, the present disclosure provides compounds, compositions, and methods of treating biological conditions mediated by, or associated with, tyrosine kinases, including, but not limited to, Cdc2 kinase, c-Kit, c-ABL, p60src, VEGFR3, PDGFRα, PDGFRβ, PDGFR-αα, PDGFR-ββ, PDGFR-αβ, FGFR3, FLT-3, Fyn, Lck, Tie-2, FMS (CSF-IR), KDR, EphA2, EphA3, EphA8, FLT1, FLT4, HCK, PTK5, RET, SYK, DDR1, and DDR2. In some embodiments, the disease or condition mediated by, or associated with, one or more kinases, e.g., tyrosine kinases, is mediated by a RTK, such as, e.g., PDGFR, PDGFR-αα, PDGFR-ββ, PDGFR-αβ, or c-Kit, or combinations thereof.

The disease or condition mediated by, or associated with, one or more kinases of the present disclosure, includes, but is not limited to, PAH, primary PAH, idiopathic PAH, heritable PAH, refractory PAH, BMPR2, ALK1, endoglin associated with hereditary hemorrhagic telangiectasia, endoglin not associated with hereditary hemorrhagic telangiectasia, drug-induced PAH, and toxin-induced PAH, PAH associated with or secondary to one or more of systemic sclerosis, mixed connective tissue disease, cancer, refractory cancer, metastatic cancer, neoplasia, hypoplasia, hyperplasia, dysplasia, metaplasia, prosoplasia, desmoplasia, angiogenic disease, pulmonary function disorders, cardiovascular function disorders, HIV infection, hepatitis, portal hypertension, pulmonary hypertension, congenital heart disease, hypoxia, chronic hemolytic anemia, newborn persistent pulmonary hypertension, pulmonary veno-occlusive disease (PVOD), pulmonary capillary hemangiomatosis (PCH), left heart disease pulmonary hypertension, systolic dysfunction, diastolic dysfunction, valvular disease, lung disease, interstitial lung disease, pulmonary fibrosis, schistosomiasis, chronic obstructive pulmonary disease (COPD), sleep-disordered breathing, alveolar hypoventilation disorders, chronic exposure to high altitude, developmental abnormalities, chronic thromboembolic pulmonary hypertension (CTEPH), pulmonary hypertension with unclear multifactorial mechanisms, hematologic disorders, myeloproliferative disorders, splenectomy, systemic disorders, sarcoidosis, pulmonary Langerhans cell histiocytosis, lymphangioleimoyomatosis, neurofibromatosis, vasculitis, metabolic disorders, glycogen storage disease, Gaucher disease, thyroid disorders, tumoral obstruction, fibrosing mediastinitis, and chronic renal failure on dialysis.

In one aspect, the present disclosure provides a method of treating pulmonary arterial hypertension (PAH) in a subject or a biological condition associated with PAH in a subject by administering to the subject a therapeutically effective amount of a compound of Structure 1, a tautomer of the compound, a pharmaceutically acceptable salt of the compound, a pharmaceutically acceptable salt of the tautomer, or a mixture thereof, wherein a compound of Structure 1 is described herein. See, e.g., Summary.

In some embodiments, the disease or condition mediated by, or associated with, one or more kinases of the present disclosure is selected form the group consisting of PAH, primary PAH, idiopathic PAH, heritable PAH, refractory PAH, drug-induced PAH, toxin-induced PAH, and PAH associated with one or more secondary diseases.

Pulmonary arterial hypertension (PAH) is a life-threatening disease characterized by a marked and sustained elevation of pulmonary artery pressure. The disease results in right ventricular (RV) failure and death. Current therapeutic approaches for the treatment of chronic pulmonary arterial hypertension mainly provide symptomatic relief, as well as some improvement of prognosis. Although postulated for all treatments, evidence for direct antiproliferative effects of most approaches is missing. In addition, the use of most of the currently applied agents is hampered by either undesired side effects or inconvenient drug administration routes. Pathological changes of hypertensive pulmonary arteries include endothelial injury, proliferation and hyper-contraction of vascular smooth muscle cells (SMCs). PAH patient status, moreover, can be assessed in accordance with the World Health Organization (WHO) classification (modified after the New York Association Functional Classification) as known in the art (Classes I-IV).

In some embodiments, the compounds of Structure 1 treat or prevent PAH in patients who failed prior therapy, especially after receiving at least one prostanoid, endothelin antagonist or PDE V inhibitor. In other embodiments, the compounds treat or prevent PAH in patients who are more severely affected, in particular in patients with Class II to Class IV functional status, or more severely Class III or IV functional status. In further embodiments, the compounds treat or prevent PAH in patients who are harboring BMPR2 mutations.

The present disclosure provides methods of preventing or treating subjects afflicted with idiopathic or primary pulmonary hypertension, familial hypertension, pulmonary hypertension secondary to, but not limited to, connective tissue disease, congenital heart defects (shunts), pulmonary fibrosis, portal hypertension, HIV infection, sickle cell disease, drugs and toxins, e.g., anorexigens, cocaine, chronic hypoxia, chronic pulmonary obstructive disease, sleep apnea, and schistosomiasis, pulmonary hypertension associated with significant venous or capillary involvement (pulmonary veno-occlusive disease, pulmonary capillary hemangiomatosis), secondary pulmonary hypertension that is out of proportion to the degree of left ventricular dysfunction, and/or persistent pulmonary hypertension in newborn babies, especially in subjects that previously failed prior PAH therapy.

In one aspect, the present disclosure provides a compound of Structure 1, a tautomer of the compound, a pharmaceutically acceptable salt of the compound, a pharmaceutically acceptable salt of the tautomer, or a mixture thereof, for treating one or more diseases associated with hyperproliferation, neoplasia, hypoplasia, hyperplasia, dysplasia, metaplasia, prosoplasia, desmoplasia, angiogenesis, inflammation, pulmonary function, and cardiovascular function, wherein a compound of Structure 1 is described herein. See, e.g., Summary.

Hyperproliferative, immunological and inflammatory, metabolic, and vascular diseases, are known in the art, and such diseases, as described in U.S. Provisional Patent No. 61/751,217, which is hereby incorporated by reference in its entirety, are therapeutic targets for the compounds and agents described herein.

Another aspect of the present disclosure related to a method of preventing or reducing elevated pulmonary pressure in a subject, by administering to the subject a therapeutically effective amount of a compound of Structure 1, a tautomer of the compound, a pharmaceutically acceptable salt of the compound, a pharmaceutically acceptable salt of the tautomer, or a mixture thereof, wherein a compound of Structure 1 is described herein. In some embodiments, the compounds of Structure 1 treat or prevent a biological condition associated with PAH, such as, e.g., abnormal: right ventricular systolic pressure (RVSP); pulmonary pressure; cardiac output; right ventricular (RV) hypertrophy; and pulmonary arterial (PA) hypertrophy.

In some embodiments, the compounds of Structure 1 reduce pulmonary pressure associated with an increase in one or more of right ventricular (RV) function, pulmonary artery (PA) systolic pressure, and/or cardiac output in the subject compared to the subject prior to the administering. In some embodiments, the reduction in pulmonary pressure is associated with a decrease in one or more of RV hypertrophy, PA hypertrophy, RVSP, sustained PA pressure, and the risk of stroke in the subject compared to the subject prior to the administering. In some embodiments, the decrease is at least a 5, 10, 15, 20, 25, 30, 35, 40, 45, 50, 55, 60, 65, 70, 75, 80, 85, 90, or 95% decrease. In some embodiments, the decrease is at least a 40% decrease. A reduction in pulmonary pressure, in some embodiments, is not associated with decreased lung function and/or increased systemic BP in the subject compared to prior to the administering.

In one aspect, the present disclosure provides a method of treating pulmonary arterial hypertension (PAH) in a subject by modulating kinase phosphorylation-state ratios (PSR) or kinase receptor activity in the subject by administering to the subject a compound of Structure 1, a tautomer of the compound, a pharmaceutically acceptable salt of the compound, a pharmaceutically acceptable salt of the tautomer, or a mixture thereof, wherein the kinase or the receptor kinase is PDGF, PDGFR, STAT3, ERK1, and/or ERK2, and wherein the compound of Structure 1 is described herein. See, e.g., Summary. Phosphorylation state profiles for proteins, kinases, kinase receptors, can be ascertain using techniques known in the art, such as, for example, Z-lyte kinase assays, Invitrogen Select Screen®, and other known kinases assays.

In suitable embodiments, the modulation of the kinase receptor activity is an inhibition of the kinase receptor activity. PDGFR, i.e., PDGFR-α, PDGFR-β, PDGFR-αα, PDGFR-ββ, and PDGFR-αβ, and/or c-Kit are examples of RTKs that are inhibited in some embodiments of the present invention. In some embodiments, the inhibition is at least a 0.001, 0.01, 0.1, 1, 5, 10, 15, 20, 25, 30, 35, 40, 45, 50, 55, 60, 65, 70, 75, 80, 85, 90, or 95% inhibition. In some embodiments, the PSR modulation is a modulation of one or more of STAT3, ERK1, ERK2, PDGF, and PDGFR i.e., PDGFR-αα, PDGFR-ββ, and PDGFR-αβ. In some embodiments, the modulation of PSR is a decrease of phosphorylated STAT3 to total STAT3 in the subject compared to the PSR in the subject prior to the administering. In some embodiments, the decrease is at least a 0.001, 0.01, 0.1, 1, 5, 10, 15, 20, 25, 30, 35, 40, 45, 50, 55, 60, 65, 70, 75, 80, 85, 90, or 95% decrease.

In some embodiments, the modulation of PSR is a decrease of diphosphorylated ERK1 to total ERK1 in the subject compared to the PSR in the subject prior to the administering. In some embodiments, the decrease is at least a 0.001, 0.01, 0.1, 1, 5, 10, 15, 20, 25, 30, 35, 40, 45, 50, 55, 60, 65, 70, 75, 80, 85, 90, or 95% decrease. In other embodiments, the modulation of PSR is a decrease of diphosphorylated ERK2 to total ERK2 in the subject compared to the PSR in the subject prior to the administering. In some embodiments, the decrease is at least a 0.001, 0.01, 0.1, 1, 5, 10, 15, 20, 25, 30, 35, 40, 45, 50, 55, 60, 65, 70, 75, 80, 85, 90, or 95% decrease.

In some embodiments, the modulation of PSR is a decrease of monophosphorylated ERK1 to total ERK1 in the subject compared to the PSR in the subject prior to the administering. In some embodiments, the decrease is at least a 0.001, 0.01, 0.1, 1, 5, 10, 15, 20, 25, 30, 35, 40, 45, 50, 55, 60, 65, 70, 75, 80, 85, 90, or 95% decrease. In some embodiments, the modulation of PSR is a decrease of phosphorylated PDGFR to total PDGFR in the subject compared to the PSR in the subject prior to the administering. In some embodiments, the decrease is at least a 0.001, 0.01, 0.1, 1, 5, 10, 15, 20, 25, 30, 35, 40, 45, 50, 55, 60, 65, 70, 75, 80, 85, 90, or 95% decrease.

Sample collection and preparation may be necessary for executing the embodiments described herein. The test samples disclosed herein are represented by, but not limited in any way to, sputum, blood (or a fraction of blood such as plasma, serum, or particular cell fractions), lymph, mucus, tears, saliva, urine, semen, ascites fluid, whole blood, and biopsy samples of body tissue. Methods of obtaining test samples are well known to those of skill in the art and include, but are not limited to, aspirations, tissue sections, swabs, drawing of blood or other fluids, surgical or needle biopsies, and the like.

### Diagnosis of Disease States

A subject's response to the administration of one or more of the compounds of Structure 1 may indicate a disease state. Association between a pathological state (e.g., cancer and/or chemotherapy drug-resistance) and the failure to obtain a positive response can be readily determined by comparative analysis in a normal population and an abnormal or affected population. Thus, for example, one can study kinase phosphorylation-state ratios (PSR), kinase receptor activity, pulmonary pressure, the presence or existence of a disease and/or disease associated with a RTK, and/or degree of disease regression, alleviation, remission, or and other biomarkers of disease associated therewith, etc., in the subject by administering to the subject a compound of Structure 1 and subsequently measuring the outcome and or biomarker response or index. In some embodiments, the PSR of PDGF, PDGFR, STAT3, ERK1, and ERK2 in both normal populations and a population affected with a particular pathological state are ascertained. The study results can be compared and analyzed by statistical means. Any detected statistically significant difference in the two populations would indicate an association.

Once an association is established between a biomarker level, e.g., PSR and/or treatment resistance, and a pathological state, then the particular physiological state can be diagnosed or detected by determining whether a patient has the particular aberration, i.e. elevated or reduced biomarker levels, e.g., PSR and/or degree of treatment resistance. The associated level can be used in conjunction with clinical factors other than the biomarker, e.g., PSR and/or treatment resistance, to diagnose a disease. Clinical factors of particular relevance in the diagnosis of cancer include, but are not limited to, the patient's medical history, a physical examination of the patient, complete blood count, cytogenetics, etc.

### EXAMPLES

The present invention is further illustrated by the following examples, which should not be construed as limiting in any way. The following is a description of the materials and methods used throughout the examples, which illustrates that RTK signaling pathways are activated in human disease conditions, e.g., pulmonary arterial hypertension (PAH), and in animal models of the disease.

Materials.

PK10453, (S)—N-(3-(1-((6-(4-hydroxy-3-methoxyphenyl)pyrazin-2yl)amino)ethyl)phenyl)-5-methylnicotinamide, was synthesized by Organix, Inc. (Woburn, Mass.). Human PA smooth muscle cells and cell culture media were obtained from Cell Applications, Inc. PDGFββ, para-toluene sulfonic acid, ammonium hydroxide, and IR780 were obtained from Sigma Aldrich (St. Louis, Mo.). Anti-phosphERK1/2, anti phosphoSTAT3, and total STAT3 antibodies were obtained from Cell Signaling Technologies, (Waltham, Mass.). Anti-total ERK1/2 antibody was obtained from Protein Simple (CA). Anti von-Willebrand Factor and actin antibodies were obtained from AbCam (Cambridge, Mass.). Antibodies against PDGFR-beta (sc-432) and p-PDGFR-beta (Tyr 1021) (sc-12909) were obtained from Santa Cruz Biotechnology (CA).

In Vitro Kinase Assay.

A Z-lyte kinase assay was performed to determine the inhibition of PDGFRalpha and PDGFRbeta mediated phosphorylation by PK10453. Ten point titration curves were modeled to calculate the IC50 (Invitrogen Select Screen®).

PASMC Proliferation Assay.

Human pulmonary artery smooth muscle cells were obtained from Cell Applications (San Diego, Calif.) and grown to 50% confluence in a 96 well format. The cells were switched to serum free media 24 hours prior to stimulation with PDGF BB 50 ng/ml and varying concentrations of PK10453. After 24 hours of treatment, a Cyquant NF Cell proliferation assay was performed (Invitrogen®), and the fluorescent signal was measured with a Cytofluor Plate reader. Data is based on an avg. of 8 replicates at each conc.

Animals.

Male Sprague Dawley rats (weight 320-330 grams; Taconic Inc.) were used for this study. Animals were housed in standard rat cages with a 12 hour light/dark cycle, and standard rat chow and water were provided ad libitum. Animals were cared for and used in accordance with the NIH Guide for the Care and Use of Animals. All animal protocols were approved by the Bassett Medical Center and Pulmokine IACUC.

Formulation and Aerosol Delivery.

PK10453 was dissolved at a concentration of 20 mg/ml in IM tosylic acid. Nebulization was performed with a PARI Nebulizer with an air pressure of 12.5 psi. The aerosol droplets were neutralized by ammonia vapor that was passed into the aerosol air stream. The particles were then dried by flowing through an annular ring of silica bead cartridges prior to reaching the exposure chamber. The 6-port exposure chamber was a nose-only exposure system custom designed and built by Powerscope Inc. (Minneapolis, Minn.). The vacuum flow rate at each port was separately controlled by a flow meter. The aerosol particle size was measured at the exit port of the drying column with an Anderson (Mark II) cascade impactor. The mass median aerodynamic diameter (MMAD) was 2 μm and the associated geometric standard deviation (GSD) was 1.6.

Estimation of Inhaled Dose.

Filters exposed to PK10453 for either 4 or 8 minutes (n=6 each group) via the Powerscope exposure chamber were placed in amber glass vials. Twelve (12) milliliters of 1:3 (v/v) methanol:acetonitrile were added to each vial containing a filter for approximately 1 hr, with periodic mixing, followed by sonication for 60 seconds. An aliquot was then diluted 100-fold by adding 10 μL of unknown filter extract to 990 μL of 1:3 (v/v) methanol:acetonitrile. Samples were vortex mixed for 30 seconds, and then a 100 μL diluted aliquot was combined with 100 μL of 172 ng/mL of a nonchemically related internal standard (PK18855) in 1:1 methanol:water, vortex mixed and transferred to autosampler vials for LC-MS/MS analysis. Filter extracts were compared against a calibration curve prepared in 100% methanol (PharmOptima®, Inc.). The aerosol concentration of PK10453 in g/liter of air was calculated based on the average total μg of PK10453 on the filters for the 4 and 8 minute exposure times, and the flow rate past each filter (0.8 L/min). The inhaled dose was calculated with the average concentration of PK10453/cm2 filter paper (average of 4 minute and 8 minute exposures), the average minute ventilation measured by plethysmography (0.15 L/min), and an estimated deposition fraction of 0.1.

Imaging.

The spatial distribution of inhaled PK10453 in the lung was evaluated by fluorescent imaging. For this study, a near IR fluorescent tracer, IR-780, was added to the drug solution in the nebulizer. In this way, the dried aerosol particles contained both the drug and IR tracer. After a two minute exposure, animals were placed under general anesthesia underwent intubation via tracheostomy, and the lungs were excised. OCT/PBS was infused via the pulmonary artery, the lung insufflated with air, and the lungs frozen in the vapor phase of liquid nitrogen. Serial approximate 2 mm sections of the lung were made and imaged on a Licor Odyssey Imager.

Pharmacokinetic Studies.

PK10453 was administered intravenously or by inhalation to animals, which were then euthanized at time 0, 10, 20, and 60 minutes (n=3 each time point). Blood samples were taken by cardiac puncture, and the lungs excised. The lungs were homogenized and PK10453 extracted with a 1:3 mixture of acetonitrile:methanol. Similarly, plasma was extracted with a 1:3 mixture of acetonitrile:methanol. Drug was assayed by LC MS/MS (PharmOptima Inc., Portage Mich.). First order exponential curves were fit to the data with Excel. The AUC was determined with the trapezoidal method of integration.

Efficacy Study in the Rat MCT Model.

Male Sprague Dawley rats received MCT 60 mg/kg IPMCT, and after 3 weeks, PK10453 or vehicle control were administered by inhalation. Four groups were studied: vehicle control (4 min exposure) and three treatment groups of PK10453 with exposure times 2 minutes (D2), 4 minutes (D4), or 8 minutes (D8) three times a day. These regimens were administered for two weeks. The vehicle consisted of aerosolized IM tosylic acid neutralized with ammonia vapor as described above. The pH of a solution prepared by dissolving captured aerosol particles in water was measured for every dose and was consistently in the range of 5.5-6.0. At the end of the study, the RV systolic pressure (RVSP) was measured, and the heart chambers dissected and weighed.

Efficacy Study in the Rat MCT+PN Model.

Pneumonectomy and implantation of a TRM53P telemetry monitor in the pulmonary artery (Telemetry Research, New Zealand and ADInstruments, Colorado) was carried out in rats. Two weeks after MCT, PK10453 was administered three times daily for 1 week. Dosing was begun 2 weeks after MCT rather than 3 weeks, because in this more aggressive model the animals developed PAH more quickly and developed distress sooner than in MCT only treated animals (Pulmokine unpublished data). The two groups underwent 4 minute exposures of either the vehicle control or PK10453. Sampling of PA pressure was performed 5 minutes before each morning dose in ambulatory animals in room air (est. pressure 716 mm Hg based on elev. of animal facility).

Measurement of PV Loops.

In a separate cohort of animals, the MCT+PN model was developed as described above, and PK10453 was then administered for 4 minutes or 8 minutes three times a day to the drug treated group. The vehicle control group underwent 4 minute exposures three times a day. Pressure Volume (PV) loops were obtained with an admittance system (Scisense, Inc.) after 14 days of treatment, while rats were under general anesthesia with isoflurane and 100% FiO₂.

Systemic Blood Pressure Study.

The effect of PK10453 on systemic BP was studied in ambulatory MCT treated rats with DSI PAC40 transmitters implanted in the descending aorta. Three weeks after administration of MCT 60 mg/kg IP, animals inhaled PK10453 or vehicle three times/day with 4 minute exposure each dose for 7 days. Blood pressure was recorded before each morning dose.

Plethysmography.

Plethysmography was performed with an EMKA dual chamber plethysmograph and IOX software. Parameters measured included breathing frequency, tidal volume, minute ventilation, peak inspiratory and expiratory flow, and airway resistance (SRaw). Animals were acclimatized to the plethysmograph for three days prior to first data acquisition. Measurements were made prior to the first dose of drug and at the study's end.

Histology and Morphometric Analysis.

At the end of the study, the heart and lungs were removed from ventilated animals under general anesthesia. Heparinized saline was infused under pressure through the main pulmonary artery. The right upper lobe was immediately tied off and placed in liquid nitrogen for Western blot and NanoPro 100 assay analysis. The heart was removed, and the RV free wall, interventricular septum and LV free wall dissected and weighed. Buffered formalin (10%) was infused under pressure both through the pulmonary artery and the trachea. Morphometric analysis was performed on H&E stained formalin fixed tissue sectioned at 8 μm. The media area and lumen area of pulmonary arterioles were measured with Image J software by a technician blinded to treatment group. Measurements were made on 20 pulmonary arterioles per section. The ratio of the lumen area to the total media area was determined. This ratio normalizes the variation in total pulmonary arteriole area.

NanoPro Immunoassay.

Relative differences in phosphorylated ERK1/2 and STAT isoforms were measured with a NanoPro100® immunoassay system (Protein Simple/Cell Biosciences, CA). See Fan et al., “Nanofluidic proteomic assay for serial analysis of oncoprotein activation in clinical specimens.” Nat Med 15:566-571 (2009).

Immunohistochemistry.

Antigen retrieval was performed with citrate buffer (pH 6.0) or Tris-EDTA buffer (pH 9.0). Immunohistochemistry was performed for the following targets: CD20 (a B cell marker), CD3 (a T cell marker), von Willebrand Factor (vWF), total STAT3, phosphoSTAT3 (Tyr705), total PDGFR-alpha, total PDGFR-beta, and phosphoPDGFR-beta. Competing peptides were available for PDGFR-alpha and phospho-PDGFR-beta. Signal detection was performed with an EXPOSE HRP/DAB kit (Abcam®).

Statistical Analysis.

Data are presented as mean±SEM unless otherwise noted. The General Linear Model with the Bonferroni correction for multiple group comparisons was used (SPSS 14.0). Significance was set at the p=0.05 level.

### Example 1

### Characterization of PK10453

An in vitro kinase assay demonstrated the IC50 at ATP Km was 21 nM for the PDGFR alpha receptor and 15 nM for the PDGFR beta receptor. PK10453 inhibited PDGF BB stimulated human PA SMC proliferation with an IC50 less than 0.5 μM.

Estimated Inhaled Dose.

The average concentration of PK10453 was 62.4±3.3 μg/cm² filter paper for the four minute exposure, and 137±7.0 μg/cm² for the 8 minute exposure, which resulted in an aerosol concentration of 91.65 μg/L air for the four minute exposure and 100.6 μg/L air for the eight minute exposure. The average inhaled dose, assuming a deposition fraction of 0.1 and rat weight 300 g, was approximately 18 μg/kg for the four minute exposure and 40 μg/kg for the eight minute exposure which suggests a linear dose, exposure time relationship (see Table 1). The estimated inhaled dose was calculated from the measured concentration of PK10453 in the aerosol, the measured minute ventilation (MV), and the estimated deposition fraction of 0.1, and rat weight 300 g, as detailed below.

TABLE 1

| | | | | | Total | Lung | Lung |
|---|---|---|---|---|---|---|---|
| Aerosol Conc | Exposure | MV | MV*Expo- | Deposition | Inhaled | Deposited | deposited |
| PK10453 μg/L | Min | L/min | sure time | fraction | μg | μg | μg/kg |
| 91.65 | 4.00 | 0.15 | 0.60 | 0.10 | 54.99 | 5.50 | 18.33 |
| 100.6 | 8.00 | 0.15 | 1.20 | 0.10 | 120.72 | 12.07 | 40.24 |

Lung Distribution and Pharmacokinetics of Inhaled PK10453.

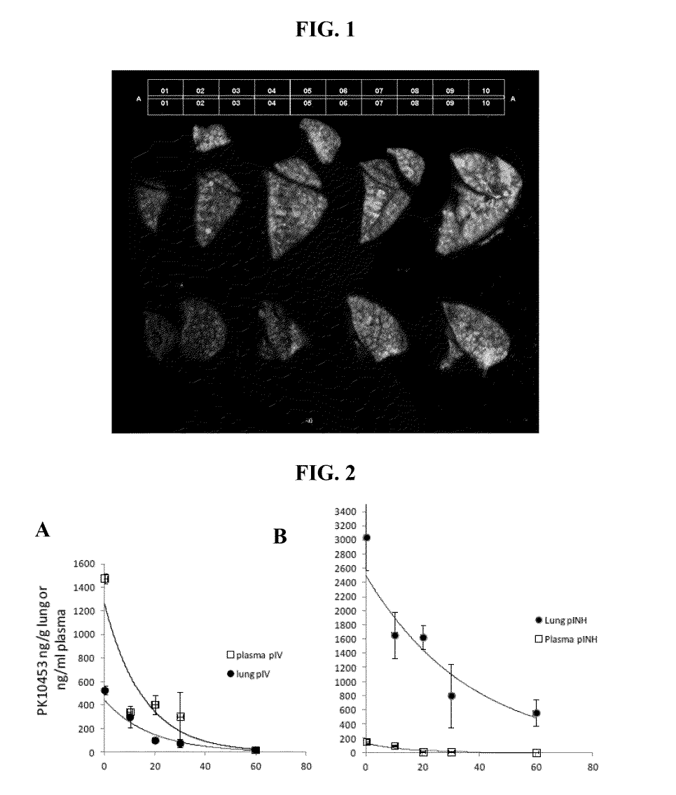

Fluorescent images of the lung sections following inhalation of PK10453 with IR780 tracer are shown in FIG. 1, where the flurorescence intensity is shown to be well distributed throughout the lungs. The network of darker lines arises from the connective tissue and therefore does not represent the airways affected by the disease.

For the pharmacokinetic study, the concentration of PK10453 in lung when administered by inhalation was compared to the concentration achieved with IV administration. As described in Morén “Aerosols in medicine: principles, diagnosis, and therapy.” Amsterdam; New York: Elsevier. xx, 429 (1993) and Phalen et al., “Inhalation exposure methodology.” Environ Health Perspect 56:23-34 (1984)), it is possible to estimate the pharmacokinetic advantage of inhalation relative to intravenous administration, R_{d}, by comparing the areas under the curve (AUC) of a plot of the drug concentration as a function of time following respiratory and intravenous (IV) administration:

R_{d}=[(AUC_{lung}/AUCₚₗₐₛₘₐ)respiratory]/[(AUC_{lung}/AUCₚₗₐₛₘₐ)IV]

The pharmacokinetic data was modeled to a first order exponential curve, and the AUC calculated from the curves (see Table 2). FIG. 2 shows the drug level in lung and plasma as a function of time following inhalation or IV administration of PK10453. The data indicate a 45 fold advantage of inhaled compared to IV administered PK10453 (R_{d}=44.6).

TABLE 2

| Y = AEXP(-bX) | A (ng/g lung) | b (min−1) | R2 |
|---|---|---|---|
| Lung (INH) | 2498 | 0.03 | 0.89 |
| Plasma (INH) | 132.7 | 0.07 | 0.93 |
| Lung (IV) | 440 | 0.06 | 0.96 |
| Plasma (IV) | 1260 | 0.07 | 0.92 |

| | AUC |
|---|---|
| Lung (INH) | 1001.82 |
| Plasma (INH) | 65.47 |
| Lung (IV) | 211.89 |
| Plasma (IV) | 617.25 |
| | Rd |
| | 44.58 |

### Example 2

### MCT Model Efficacy

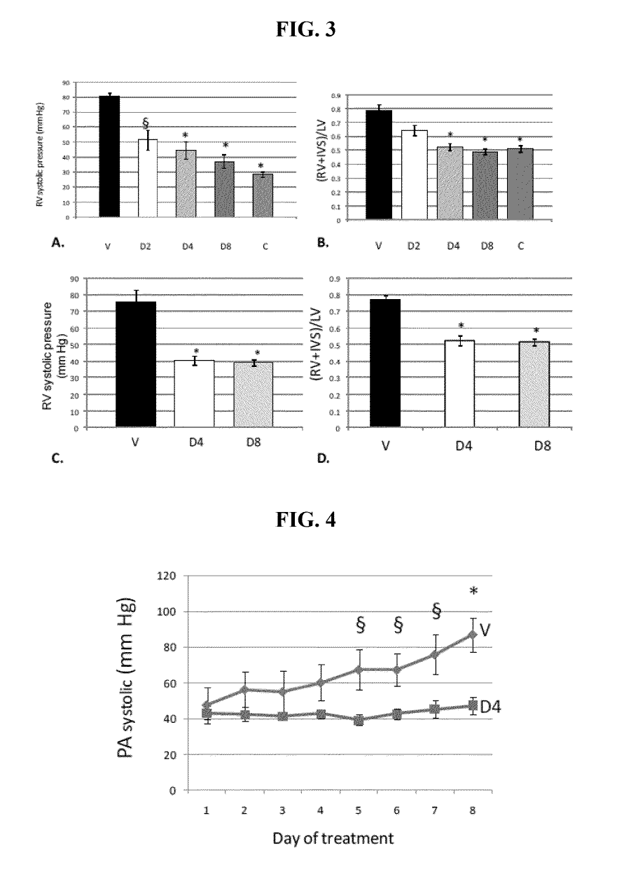

RVSP values are shown in FIG. 3A. In the vehicle group (n=6), RVSP was 80.4±2.6 mm Hg. For the treatment groups, D2 (n=6), 51.4±6.5; D4 (n=6), 44.4±3.8; and D8 (n=5), 37.1±4.5 mm Hg (p<0.001). Normal control group RVSP was 28.5±2.6 mm Hg (n=3). In the D4 group, there was a 44% reduction in RVSP, and in the D8 group, there was a 54% reduction in RVSP compared to the vehicle treated group. There was also a significant reduction in the degree of RV hypertrophy as measured by the ratio (RV+IVS)/LV weight. See FIG. 3B. The data are represented by this ratio because the septum is shared by the RV and LV. However, use of the RV/(IVS+LV) ratio also showed similar positive results.

### Example 3

### MCT+PNMCT+PN Model Efficacy

PV loop study. The RV end systolic pressure (ESP) was lower and the RV ejection fraction (EF) was higher in both the D4 and D8 treatment groups compared to vehicle control. Cardiac output in the D8 group was increased compared to the Vehicle group. See Table 3 and FIG. 3C. The study animals underwent left pneumonectomy followed 7 days later by MCT 60 mg/kg IP. Two weeks after MCT administration, PK10453 or vehicle were given by inhalation three times a day for two weeks. PV loops were acquired at the end of this period. With respect to Table 3: V=vehicle; D4=4 minute inhalation PK10453; D8=8 minute inhalation PK10453; n=4 each group; *p<0.001; **p≦0.01; §p<0.05 vs. V.

TABLE 3

| Group | Sel | HR (bpm) | ESP (mm Hg) | EDP (mm Hg) | ESV (μl) | EDV (μl) | SV (μl) | CO ml/min | EF | SW |
|---|---|---|---|---|---|---|---|---|---|---|
| V | Mean | 290 | 83.21 | 10.31 | 484.17 | 621.32 | 137.15 | 39.03 | 25.43 | 10123 |
| | SEM | 25 | 3.49 | 1.24 | 148.32 | 139.49 | 14.19 | 0.62 | 8.36 | 2698 |
| D4 | Mean | 288 | 43.20* | 2.62§ | 144.14 | 408.95 | 264.81 | 77.59 | 65.4** | 9818 |
| | SEM | 21 | 6.08 | 0.30 | 25.89 | 34.94 | 12.66 | 2.59 | 3.47 | 769 |
| D8 | Mean | 315 | 38.44* | 4.87 | 155.40 | 488.68 | 333.28** | 105.1** | 67.1** | 5481 |
| | SEM | 41 | 1.43 | 1.86 | 22.69 | 52.00 | 49.81 | 15.51 | 4.59 | 1829 |

Effect of PK10453 on RV Hypertrophy.

Treatment with PK10453 resulted in a significant decrease in RV hypertrophy in the rat MCT+PNMCT+PN model. FIG. 3D.

Telemetry Study.

The results of the telemetry study in the rat MCT+PN model are shown in FIG. 4. At day 0 prior to start of treatment, the PA systolic pressure in the vehicle groups was 41.0±11.7 mm Hg, and in the PK10453 group, was 43.1±3.5 mm Hg (p=NS). After five days of treatment, the PA systolic pressure was 69.4±12.9 mm Hg in the vehicle group and was significantly lower at 47.3±3.0 mm Hg in the PK10453 group (p<0.01). On treatment day 8, the PA systolic pressure in the vehicle group was 83.5±8.5, but significantly lower at 47.3±4.9 mm Hg in the PK10453 group (p<0.001).

### Example 4

### Analysis of Pulmonary Arteriole Histology and Morphology

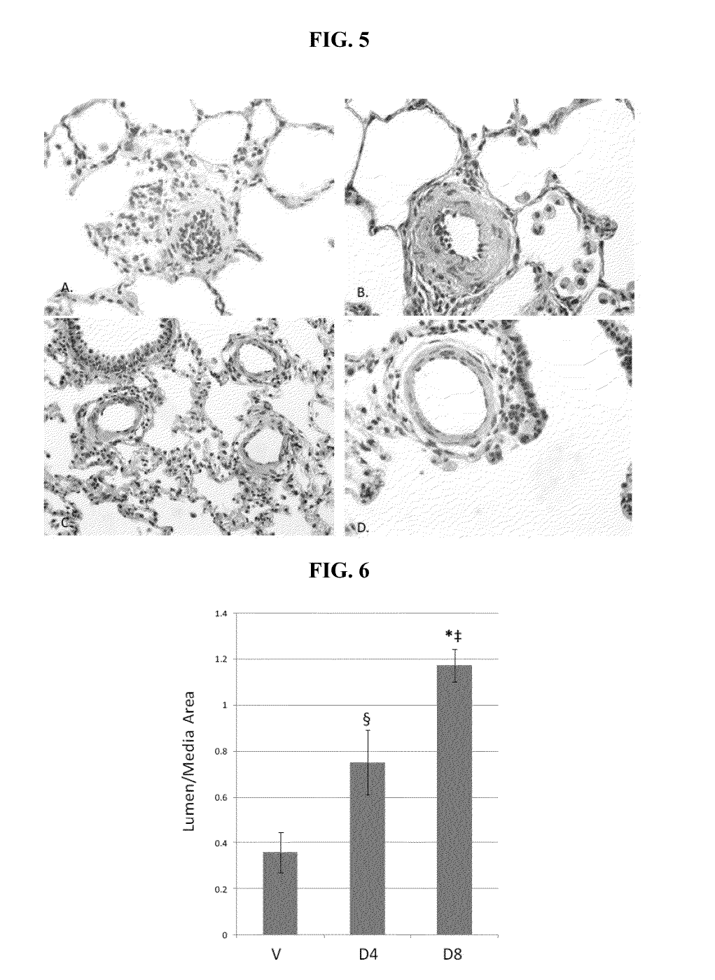

Examples of pulmonary arteriole hypertrophy and intraluminal cellular proliferative lesions are shown in FIG. 5, while the quantitative analysis is represented in FIG. 6. The lumen area to media area ratio (L/M) was significantly higher in the PK10453 treated groups compared to vehicle, where the higher dose, D8 (n=4) L/M 1.17±0.07, the lower dose, D4 L/M 0.75±0.14, and the vehicle control V (n=6): 0.36±0.09 (p=0.032 D4 vs. V; p=0.00014 D8 vs. V; p=0.028 D8 vs. D4). Immunohistochemistry with CD20, a marker of B cell lineage showed signal in intraluminal cells within pulmonary arterioles of vehicle treated animals. CD3, a marker of T cells, showed positive staining within occlusive lesions of obstructed pulmonary arterioles (to a lesser extent). There were also some perivascular CD20+ cells.

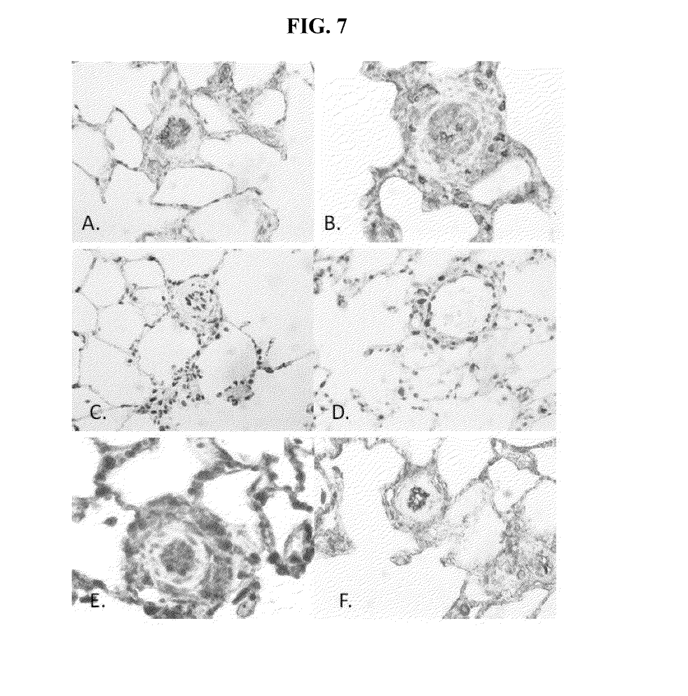

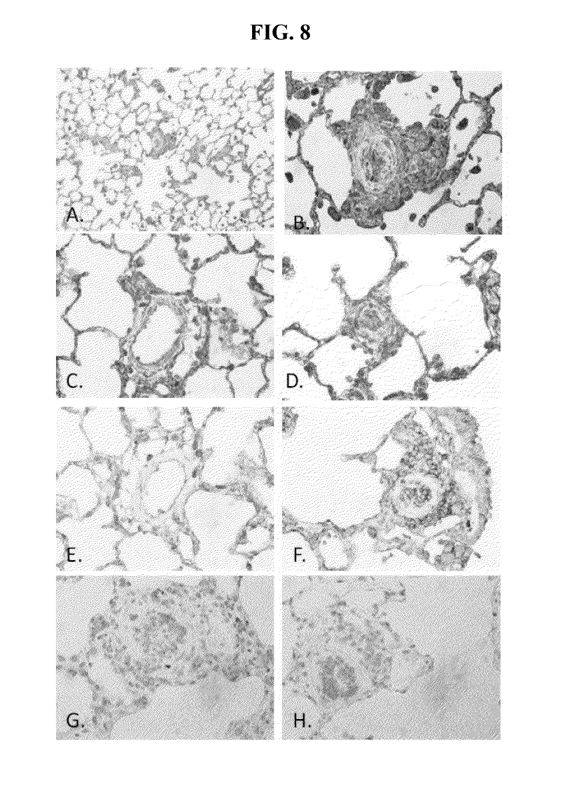

The endothelial cell marker, vWF, showed signal predominantly within the pulmonary arterioles. The tyrosine705 phosphoSTAT3 antibody showed localization of pSTAT3 to nuclei of endothelial cells and perivascular cells. See FIG. 7. Signal for PDGFR-alpha and beta was strongest around pulmonary arterioles and within the intraluminal cellular lesions as well as in pulmonary arteriolar smooth muscle cells. Phosphorylated PDGFR-beta had a cobblestone appearance in neointimal cells and perivascular cells. See FIG. 8.

### Example 5

### NanoPro® Immunoassays and Western Blots

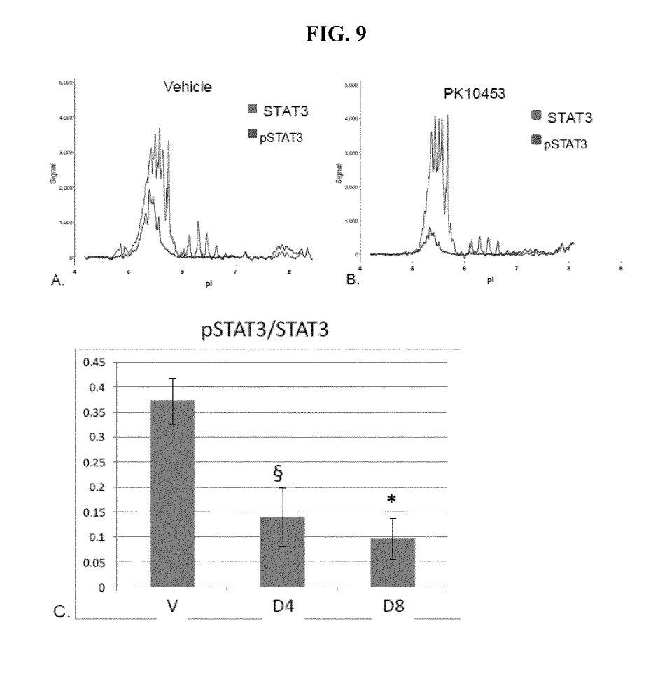

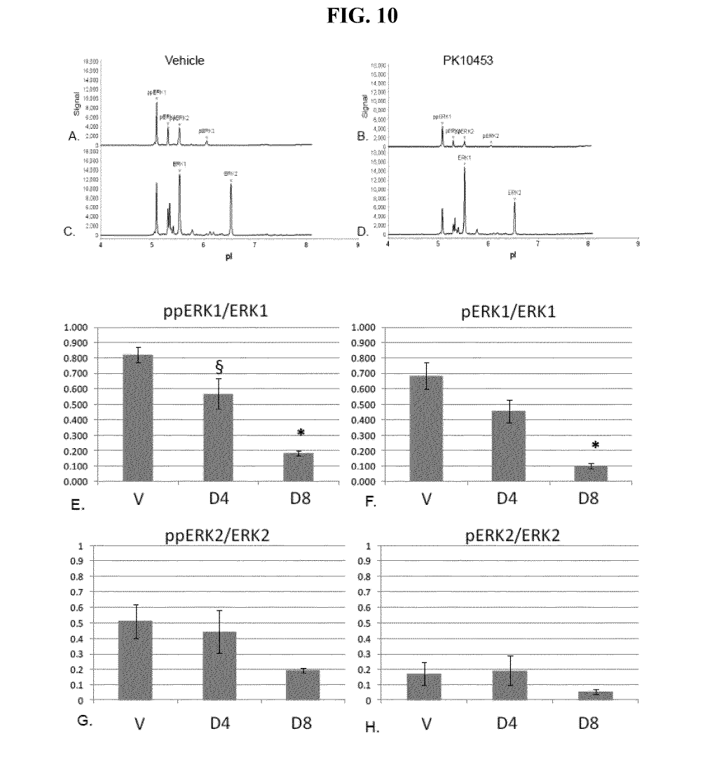

Nanopro® Immunoassays for pSTAT3/STAT3 are shown in FIG. 9. There was a significant reduction in the pSTAT3/STAT3 ratio in both the D4 and D8 groups compared to vehicle. FIG. 10 shows the effect of inhaled PK10453 on ppERK1/ERK1, pERK1/ERK1, ppERK2/ERK2 and pERK2/ERK2 in lung homogenates. There were significant reductions in ppERK1/ERK1 and pERK1/ERK1 in the D4 and D8 groups compared to vehicle.

### Example 6

### Body weights, Systemic BP, and Plethysmography Studies

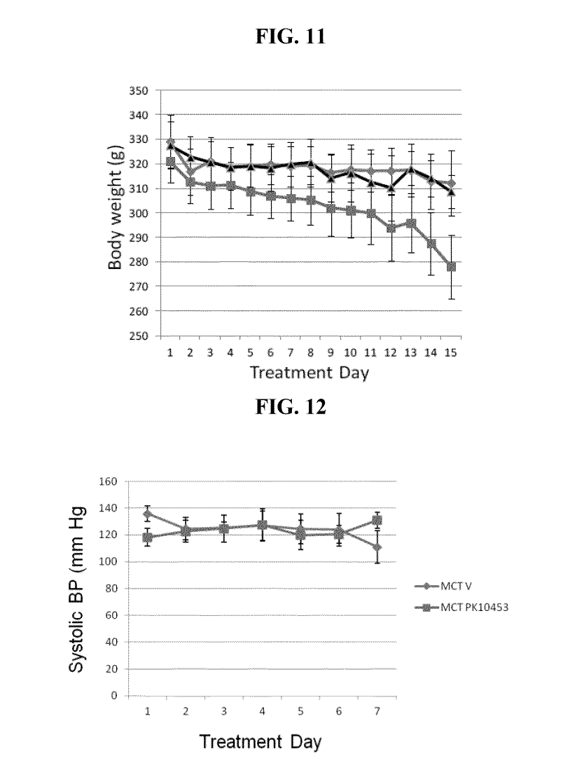

Compared to vehicle, there was a trend to a slower rate of decline in body weight in the treated vs. vehicle groups. See FIG. 11. On day seven of treatment, systolic BP was 111±21 mmHg in the MCT vehicle group (n=3) compared to 131±10 mmHg in the MCT PK10453 group (n=3), as shown in FIG. 12.

Two chamber plethysmography was measured at day 1 and day 15 of PK10453/vehicle administration in the rat MCT+PNMCT+PN model. The results are shown in Table 4. Treatment with PK10453 was associated with a slower decline in minute ventilation (MV), and a significant improvement in peak inspiratory flow (PIF) and peak expiratory flow (PEF) in the 4 minute exposure group (D4) compared to vehicle.

TABLE 4

| | | Day1 | | | | | | Day15 | | | | | |
|---|---|---|---|---|---|---|---|---|---|---|---|---|---|
| Drug Group | | PIF | PEF | TV (ml) | MV (ml/min) | f | SRaw | PIF | PEF | TV | MV | f | SRaw |
| V (n = 6) | mean | 8.81 | 9.68 | 0.86 | 193.66 | 244.79 | 40.37 | 4.97 | 5.86 | 0.52 | 107.59 | 214.43 | 38.93 |
| | sem | 0.79 | 0.98 | 0.14 | 20.66 | 28.02 | 4.11 | 0.39 | 0.44 | 0.07 | 9.58 | 13.83 | 6.53 |
| D4 (n = 5) | mean | 9.82 | 11.04 | 1.00 | 223.24 | 224.68 | 39.73 | 7.82* | 9.33* | 0.85 | 176.12§ | 217.12 | 33.09 |
| | sem | 0.70 | 0.56 | 0.07 | 11.99 | 9.87 | 3.33 | 0.34 | 0.67 | 0.12 | 14.53 | 18.64 | 4.80 |
| D8 (n = 5) | mean | 8.54 | 9.43 | 0.74 | 174.68 | 259.13 | 36.01 | 6.06 | 6.64 | 0.63 | 128.49 | 232.11 | 49.26 |
| | sem | 0.72 | 1.01 | 0.15 | 22.32 | 26.42 | 3.82 | 0.84 | 0.99 | 0.16 | 19.47 | 30.71 | 7.11 |

Abberviations: PIF: peak inspiratory flow; PEF: peak expiratory flow; TV: tidal volume; MV: minute ventilation; f: breathing frequency (breaths per minute); SRaw: airway resistance
*p < 0.01 D4 vs.V;
§p = 0.02 D4 vs. V.

### Example 7

### Molecular Modeling and Covalent RTK Inhibitors

Molecular docking was performed using homology models of the PDGFα receptor and PDGFβ receptor, which were based on the crystal structure of c-Kit (1T46). PDGFRα and PDGFRβ receptor homology to c-Kit is 59% and 63%, respectively, as shown in Table 5.

TABLE 5

| Seq | 1T46 | Alpha | Beta |
|---|---|---|---|
| 1T46 | ID | 0.639 | 0.599 |
| Alpha | 0.639 | ID | 0.760 |
| Beta | 0.599 | 0.760 | ID |

In short, NCBI BLAST search techniques were used to examine the available templates. The human platelet-derived growth factor receptor alpha subunit’ sequence has an accession number of P16234 and beta subunit: P09619 in the Uniprot database. A BLAST search was carried out in order to examine the available templates. Indeed, c-Kit tyrosine kinase (1T46) was found to possess the highest identity with the query sequences. Crystal structure of the c-Kit (1T46) used for template was obtained from the Brookhaven Protein DataBank. The initial sequence alignment of the template and query sequences were carried out using the ClustalW and resulted in −70% pair-wise sequence identity. See Table 6.

TABLE 6

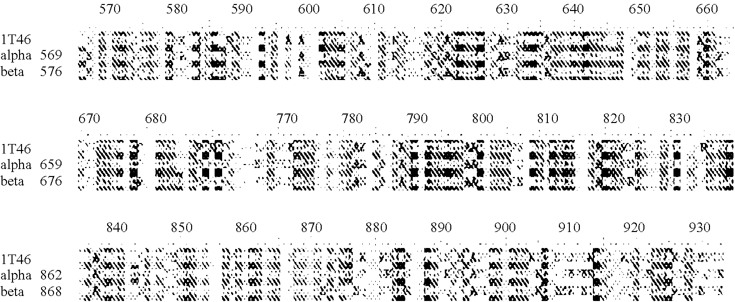

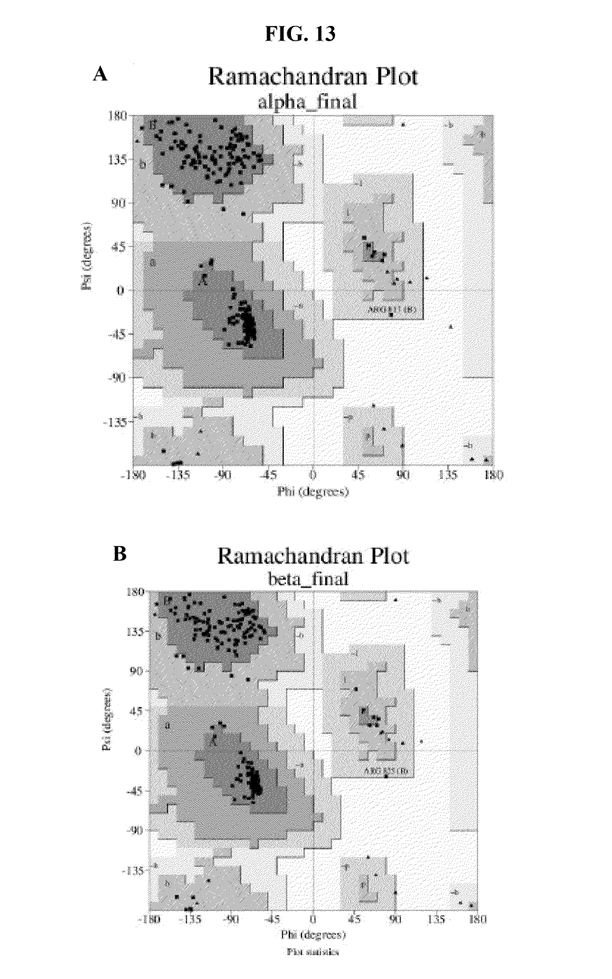

Three-dimensional (3D) models comprising all non-hydrogen atoms were generated by the MODELLER9v 11 package. A bundle of ten models from random generation of the starting structure was calculated. Each generated model was first optimized with the variable target function method (VTFM) with conjugate gradients (CG), and was then further refined utilizing molecular dynamics (MD) with simulated annealing (SA). Subsequently, the best DOPE score model was selected as final template. The quality of the model was evaluated using PROCHECK and 100.0% of the residues were found in allowed regions of the Ramachandran diagram in case of both models. See FIG. 13A-C. The total quality G-factor was 0.16 and 0.15 for alpha and beta PDGF, respectively, which is indicative of a good quality model (best models' values of the G-factor in PROCHECK are close to zero). See Laskowski et al., Journal Of Applied Crystallography 26:283-91 (1993). See FIG. 13A-C.

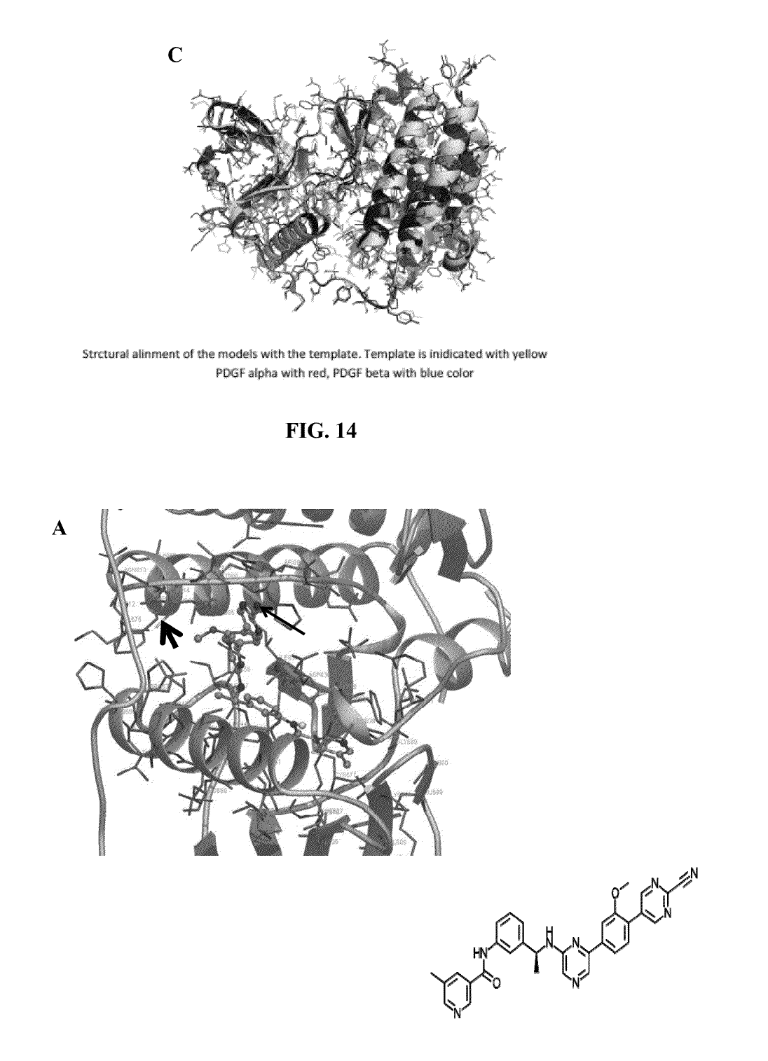

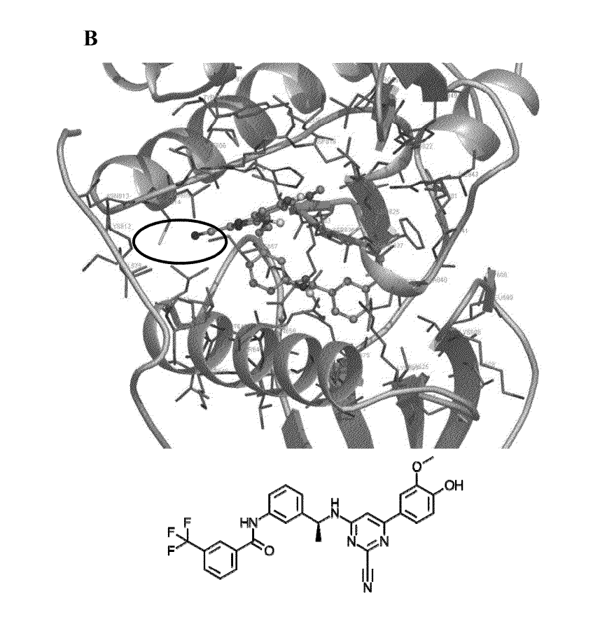

Electrophiles other than those described by Leproult et al. (2011), were employed for increased efficacy. See Barf et al. (2012) and Oballa et al., Bioorg Med Chem Lett 17:998-1002 (2007) (describing nitrile-containing electrophiles). Such electrophiles were introduced at various positions with respect to the PDGFR scaffold. The spatial orientation of the inhibitor “warheads,” relative to the target cysteine residues, were analyzed by visual inspection and distance mapping. The free energy of binding and estimated Ki were calculated for two representative compounds as shown in FIG. 14A-B. FIG. 14A shows molecular docking of PK10498 with the PDGF-alpha receptor, where the long arrow points to the warhead structure and the short arrow points to CYS814. The warhead is in suboptimal spatial orientation, where PK10498 free energy binding estimate −10.07 kcal/mol; Ki 41.79 nM. FIG. 14B shows molecular docking of PK10562 (structure shown) with PDGF-alpha receptor. Proximity of the nitrile containing warhead in proximity to CYS814 is shown (oval encircles both CYS814 and warhead). The warhead is in optimal position to form a covalent bond with CYS814. PK10562 free energy binding estimate −10.37 kcal/mol; Ki 25 nM. (Use of the CF₃ moiety improves binding energy thereby increases selectivity/specificity. The compounds with the lowest free energy of binding and closest proximity of the warhead to a cysteine residue impart irreversible selective RTK inhibitors. See Table 7 for amino acid interaction data and Table 8 for complete irreversible inhibitor molecular docking data.

TABLE 7

| | PDGF | | | |
|---|---|---|---|---|
| | receptor | Polar | Hydrophobic | |
| Compound | isoform | Interaction | Interaction | Other |
| PK10516 | Alpha | LYS627 | VAL607 | |
| (S22) | | GLU644 | MET648 | |
| | | HIS816 | LEU809 | |
| | | ASP836 | CYS814 | |
| | | | ILE834 | |
| | | | CYS835 | |
| | | | PHE937 | |
| | BETA | LYS634 | VAL614 | |
| | | GLU651 | MET655 | |
| | | HIS824 | LEU817 | |
| | | ASP844 | CYS822 | |
| | | | ILE842 | |
| PK10535 | ALPHA | LYS627 | VAL607 | VAL658 |
| (S31) | | GLU644 | ILE647 | |
| | | HIS816 | MET648 | |
| | | ARG836 | CYS814 | |
| | | | ILE834 | |
| | | | CYS835 | |
| | BETA | LYS634 | VAL614 | CYS822 |
| | | GLU651 | ALA632 | |
| | | HIS824 | MET655 | |
| | | ARG825 | CYS822 | |
| | | ASP844 | ILE842 | |
| | | | CYS843 | |
| PK10550 | ALPHA | THR874 | VAL607 | GLU644 |
| (S29) | | ARG817 | MET648 | VAL815 |
| | | | LEU651 | LEU809 |
| | | | ILE657 | HIS816 |
| | | | | CYS835 |
| | BETA | LYS634 | VAL614 | THR681 |
| | | GLU651 | ILE654 | LEU817 |
| | | ARG825 | MET655 | CYS822 |
| | | ASP826 | LEU658 | |
| PK10559 | ALPHA | GLU644 | VAL607 | LYS627 |
| (S33) | | HIS816 | MET648 | |
| | | ASP836 | LEU825 | |
| | | | ILE834 | |
| | | | CYS835 | |
| | | | PHE837 | |
| | BETA | LYS634 | ILE654 | VAL614 |
| | | GLU651 | MET655 | |
| | | THR681 | LEU658 | |
| | | HIS824 | CYS822 | |
| PK10562 | ALPHA | GLU644 | ILE647 | |
| (S30) | | HIS816 | MET648 | |
| | | ASP836 | CYS814 | |
| | BETA | LYS634 | ILE654 | VAL614 |
| | | GLU651 | MET655 | |
| | | HIS824 | CYS822 | |
| | | ASP844 | ILE842 | |
| | | | CYS843 | |

### Example 8

### Discussion and Applied Embodiments

This foregoing examples confirmed that a novel inhaled PDGF receptor inhibitor, PK10453, would decrease pulmonary hypertension both in the rat monocrotaline (MCT) model and the rat MCT plus pneumonectomy (+PN) model of PAH. As described, PK10453 delivered by inhalation, for four (D4) and eight (D8) minute exposures three times a day for two weeks, decreased right ventricular systolic pressure (RVSP) in both the rat MCT and rat MCT+PN models: vehicle MCT group (n=6) RVSP was 80.4±2.6 mm Hg; in the D4 MCT group (n=6), 44.4±3.8 mm Hg; and in the D8 MCT group (n=5), 37.1±4.5 mm Hg (p<0.001 vs. vehicle); in the vehicle MCT+PN group (n=4) RVSP was 83.2±3.5 mm Hg; in the D4 MCT+PN group (n=4), 43.2±6.1 mm Hg, and in the D8 MCT+PN group (n=4), 38.4±1.4 mm Hg (p<0.001). In the rat MCT+PN model, continuous telemetry monitoring of pulmonary artery pressures also demonstrated that PK10453 prevented progression of PAH.

Concomitant with a reduction in pulmonary pressures, there was a decrease in right ventricular (RV) hypertrophy, and pulmonary arteriolar hypertrophy. PK10453 decreased the ratio of phosphorylated STAT3 (Y705) to total STAT3, the ratio of diphosphorylated ERK1 to total ERK1 and the ratio of monophosphorylated ERK1 to total ERK1 in lung extracts from MCT+PN animals. Immunohistochemistry demonstrated phosphorylated PDGFR in neointimal and perivascular cells of pulmonary arterioles in the MCT+PN model. In conclusion, PK10453, when delivered by inhalation, significantly decreased the progression of PAH in the rat MCT and MCT+PN models.

Such results are congruent with the potential of kinase inhibitors as treatments for pulmonary arterial hypertension (PAH). The PDGF signaling pathway is activated in human idiopathic PAH (iPAH) and in animal models of the disease. PDGF-A, PDGF-B, PDGFR-alpha, and PDGFR-beta mRNA expression were increased in small pulmonary arteries from patients with iPAH compared to control subjects, and Western blot analysis showed a significant increase in protein expression of PDGFR-beta in PAH lungs. See Perros et al., Am J Respir Crit Care Med 178:81-88 (2008). The migration of PASMCs was inhibited by imatinib, a potent PDGF receptor inhibitor. Id. Imatinib also decreased RVSP and improved survival in the rat MCT model of PAH. See Schermuly et al., J Clin Invest 115:2811-2821 (2005). Reports of patients with refractory PAH observed a favorable response to imatinib. See, e.g., Souza et al., Thorax 61:736 (2006). The IMPRES trial, which examined the effect of imatinib in patients with severe PAH, showed an improvement in the 6-min walk distance and in cardiopulmonary hemodynamics. Ghofrani et al., N Eng. J Med 353:1412-13 (2005).

For the first time, however, the present invention discloses novel PDGF receptor inhibitors, PK10453, and various derivatives thereof, including various modeled irreversible derivatives, when administered by inhalation, decreased (or are predicted to decrease) the severity of PAH in two animal models of the disease: the rat MCT, and the rat MCT+PN model. Likewise, this is the first study to report efficacy of PDGF receptor inhibition in the rat MCT+PN model. A sustained reduction in PA pressure was also found in ambulatory PAH (MCT+PN) animals treated with PK10453. Concomitant with a significant reduction of PA and RV systolic pressure in these models, a reduction in RV hypertrophy and an improvement in the lumen to media ratio of pulmonary arterioles were demonstrated. Pressure volume loops displayed an improvement in RV ejection fraction, a higher cardiac output, and a trend towards lower stroke work in PK10453 treated animals compared to control animals. In lung extracts of PK10453 treated animals, there was a significant reduction in the pSTAT3/STAT3, ppERK1/ERK1 and pERK1/ERK1 ratios.

Because PAH is a disease substantially localized to the lung, direct administration of the drug to the target site, via inhalation, offers the advantage of higher local concentrations (greater efficacy) and lower systemic concentrations of drug (lower side effects). Pharmacokinetic studies demonstrated a 45 fold advantage of inhalation delivery compared to IV administration of PK10453. While PK10453 decreased RV systolic pressure by 50% in the rat MCT model, it did not have an adverse effect on systemic blood pressure. Also, inhaled PK10453 did not appear to adversely affect lung function over a 2 wk administration course.

The results are consistent with, yet superior to, prior reports that the PDGF receptor inhibitor imatinib, when delivered systemically, decreased pulmonary hypertension in the rat MCT model. Use of the rat MCT model for preclinical testing of drug candidate efficacy has recently been criticized because, in this model, animals do not develop neointimal or plexiform lesions characteristic of human PAH. The MCT+PN model, however, is a more aggressive model of PAH compared to the MCT only model, and may more accurately reflect the pathology of the human disease. See White et al. (2007) Am J Physiol. 293:583-90.

The pathologic signature of PAH consists of concentric and plexiform lesions in small precapillary pulmonary arterioles. Concentric lesions arise from the proliferation of neointimal cells, which occlude the vessel lumen. It has been reported that these concentric obstructive neointimal lesions are composed of myofibroblasts and/or endothelial cells. See, e.g., Cool et al. (1997). In addition, perivascular infiltrates, consisting of T cells, B cells, and macrophages, have been found in plexogenic PAH. Plexiform lesions are characterized by disorganized vascular channels that stain for endothelial cell markers. It has been shown that the plexiform lesions in human lung samples from patients with idiopathic/primary PAH consist of a monoclonal expansion of endothelial cells. See Lee et al. (1998). Therefore, it has been suggested that this type of PAH can be thought of as a “cancer” of pulmonary arteriolar endothelial cells, as described above. It has been proposed that in the initial stages of the disease, an acute apoptotic loss of normal endothelial cells results in emergence and clonal expansion of apoptosis resistant endothelial cells. The paradigm of PAH as a neoplastic process imparts a wide variety of disease indications vulnerable to the instant compounds.

Some information is available regarding the phenotype and characteristics of neointimal lesions in the rat MCT+PN model. The present data relates human PAH lesions with lesions found in the rat MCT+PN model. A predominance of perivascular lesions in the rat MCT+PN model is noted. The occlusive lesions stained positive for vWF and pSTAT3, consistent with prior findings in human PAH. CD20+ cells indicated the presence of B-cells within neointimal lesions. CD3+ cells were also found to a lesser extent in neointimal and perivascular cellular infiltrates. Both PDGFR-alpha and -beta were detected in perivascular, and neointimal lesions as well as cells in the pulmonary arteriolar media. Interestingly, the signal for phospho-PDGFRβ appeared to predominate in perivascular and neointimal cells.

Schermuly et al. (2010) demonstrated a reduction in phospho-ERK1/2 by imatinib in the rat MCT model of PAH, while Jasmin et al. (2006) have shown activation of STAT3 in the rat MCT model. Masri et al. (2007) found that STAT3 was activated in human idiopathic PAH. A nanofluidic proteomic immunoassay to quantify phosphorylated species of STAT3 and ERK1/2 in lungs extracts of PAH animals was employed in the present study. This assay was previously used to examine the effects of imatinib on pSTAT3, and pERK1/2 in chronic myelogenous leukemia (CML). The assay has utility in distinguishing monophosphorylated isoforms and diphosphorylated isoforms of proteins. For example, patients with CML who responded to imatinib had a distinct reduction in levels of monophosphorylated ERK2. The present data demonstrate that the ERK1 isoform and both the disphosphorylated form of ERK1 and the monophosphorylated form of ERK1 predominated in lungs of MCT pneumonectomized rats. Treatment with PK10453 significantly and selectively decreased ppERK1/ERK and pERK1/ERK1. Therefore, these results substantiate that inhaled PK10453 decreased phosphorylation cascades associated with activation of the PDGF receptor in PAH.

In conclusion, efficacy of an inhaled PDGF receptor inhibitor, PK10453, in both the MCT, and MCT+PN rat models of PAH has been demonstrated. Treatment with PK10453 was associated with a significant reduction in pulmonary arterial pressures in ambulatory animals, an improvement in right ventricular function, and a reduction in RV hypertrophy. Histologic analysis demonstrated an improvement in the pulmonary arteriole lumen to media ratio in animals treated with PK10453 and a decrease in the phosphorylation state of STAT3 and ERK1. There was no significant effect of PK10453 on systemic blood pressure, and no adverse effect of PK10453 on lung function was found. PK10453 when administered by inhalation has potential as a treatment for PAH.

TABLE 8
(Irreversible RTK Inhibitors)

| | | Kit | | | PDGFα | | | PDGFβ | | |
|---|---|---|---|---|---|---|---|---|---|---|
| Name | | | | CYS788 | | Ki | CYS | | | CYS |
| (Structure) | (Structure) | kcal/mol | ki (nM) | proximity | kcal/mol | (nM) | proximity | Kcal/Mol | ki (nM) | proximity |
| PK10465noR (S3) | 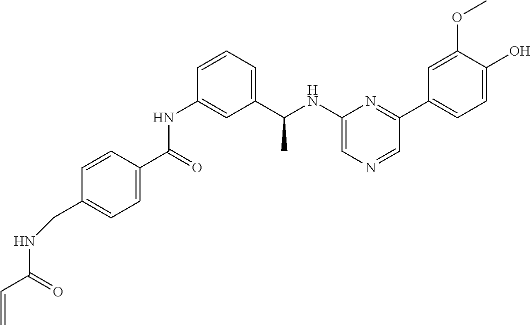 [SMILES: C=CC(=O)NCc1ccc(C(=O)Nc2cccc([C@H](C)Nc3cncc(-c4ccc(O)c(OC)c4)n3)c2)cc1] | −11.9 | 1.88 | good | −10 | 46 | 814 | −11 | 8.32 | 822 |
| PC10467 (S14) | 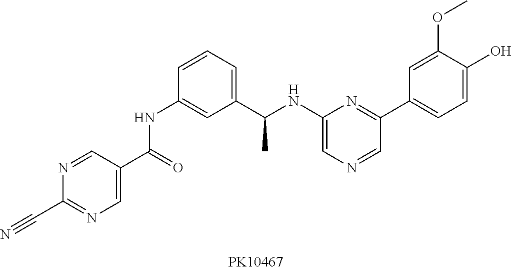 [SMILES: COc1cc(-c2cncc(N[C@@H](C)c3cccc(NC(=O)c4cnc(C#N)nc4)c3)n2)ccc1O] | −10.98 | 8.91 | good | −9.2 | 181 | 835 | −9.44 | 120 | 822 |
| PC10468 (S15) | 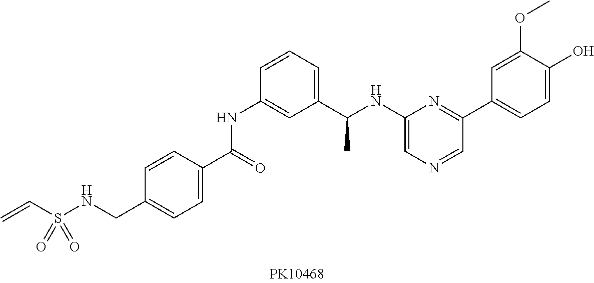 [SMILES: C=CS(=O)(=O)NCc1ccc(C(=O)Nc2cccc([C@H](C)Nc3cncc(-c4ccc(O)c(OC)c4)n3)c2)cc1] | −11 | 6.88 | very good | −9 | 205 | 835 close | −9.97 | 48 | 843 |
| PK10468PCH3 ortho (S16) | 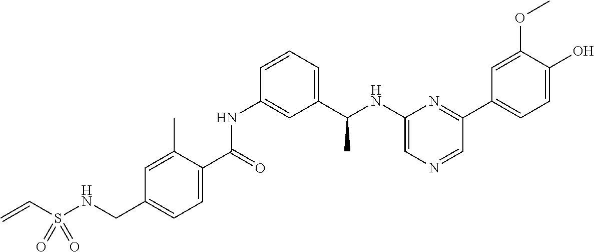 [SMILES: C=CS(=O)(=O)NCc1ccc(C(=O)Nc2cccc([C@H](C)Nc3cncc(-c4ccc(O)c(OC)c4)n3)c2)c(C)c1] | −12.42 | 785 pM | good | | | | | | |
| pk10496 (S17) | 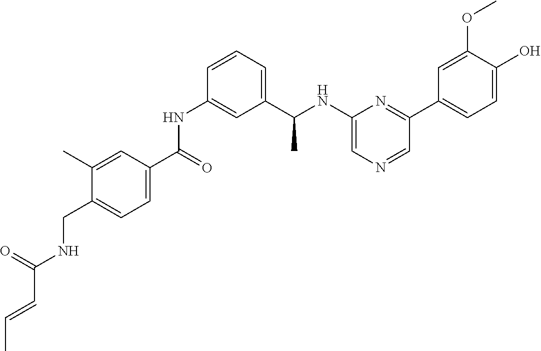 [SMILES: C/C=C/C(=O)NCc1ccc(C(=O)Nc2cccc([C@H](C)Nc3cncc(-c4ccc(O)c(OC)c4)n3)c2)cc1C] | −10.79 | | | −9.68 | 79.95 | close to 835 | −9.86 | 59 | close to 843 |
| pk10497 (S9) | 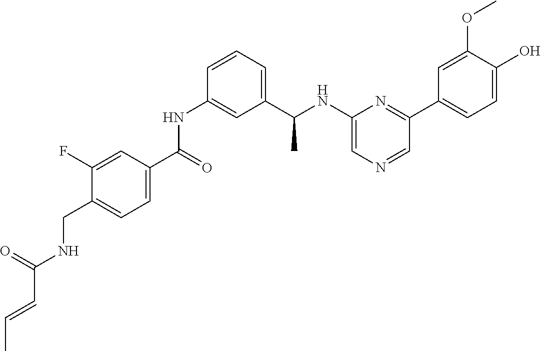 [SMILES: C/C=C/C(=O)NCc1ccc(C(=O)Nc2cccc([C@H](C)Nc3cncc(-c4ccc(O)c(OC)c4)n3)c2)cc1F] | −12.35 | | | −9.18 | 185 | 835 close | −10 | 43 | 822 |
| pk10498 (S18) | 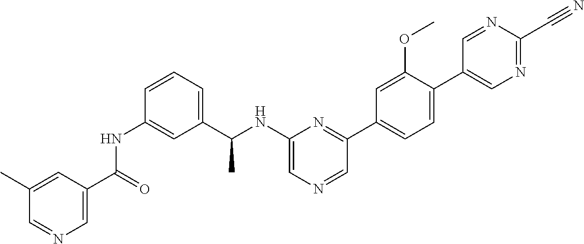 [SMILES: COc1cc(-c2cncc(N[C@@H](C)c3cccc(NC(=O)c4cncc(C)c4)c3)n2)ccc1-c1cnc(C#N)nc1] | −12.67 | | | −10.07 | 42 | 814 | −9.98 | 48 | 822 |
| pk10499 (protonated S18) | 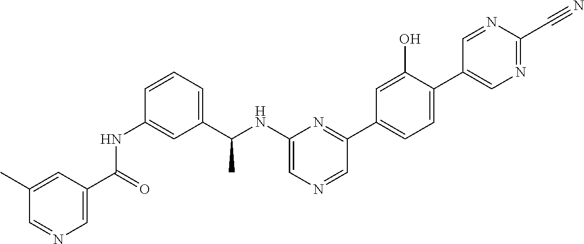 [SMILES: Cc1cncc(C(=O)Nc2cccc([C@H](C)Nc3cncc(-c4ccc(-c5cnc(C#N)nc5)c(O)c4)n3)c2)c1] | −12.69 | | | −10.7 | 14.4 | 814 | −9.69 | 78.6 | 822 |
| pk10503 (S19) | 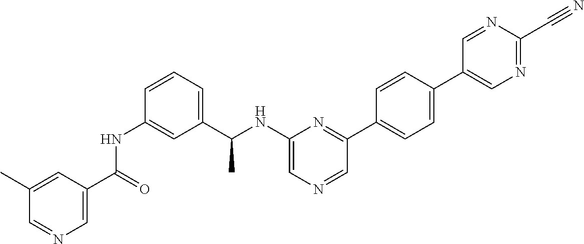 [SMILES: Cc1cncc(C(=O)Nc2cccc([C@H](C)Nc3cncc(-c4ccc(-c5cnc(C#N)nc5)cc4)n3)c2)c1] | −13.46 | | | −9.82 | 63 | 834 | −10.6 | 16 | 834 |
| pk10507 (S20) | 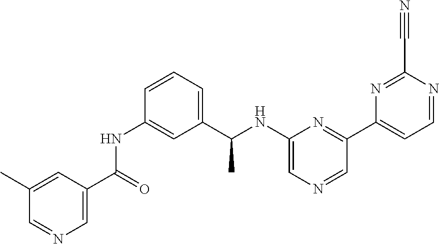 [SMILES: Cc1cncc(C(=O)Nc2cccc([C@H](C)Nc3cncc(-c4ccnc(C#N)n4)n3)c2)c1] | −11.46 | | | −9.61 | 90.4 | 677 close | −9.8 | 65 | 822 fairly close |
| pk10515 (S21) | 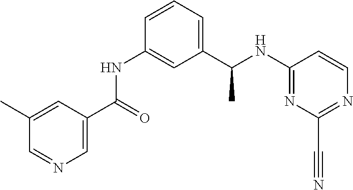 [SMILES: Cc1cncc(C(=O)Nc2cccc([C@H](C)Nc3ccnc(C#N)n3)c2)c1] | −10.23 | | | −9.1 | 193 | 814 very close | −9.28 | 94 | 834 far |
| pk10516 (S22) | 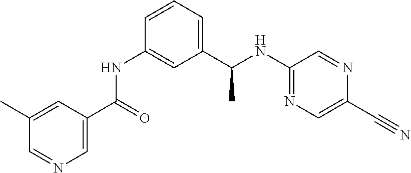 [SMILES: Cc1cncc(C(=O)Nc2cccc([C@H](C)Nc3cnc(C#N)cn3)c2)c1] | −10.25 | | | −9.1 | 214 | 814 close | −9.28 | 157 | 822 close |
| pk10517 (S26) | 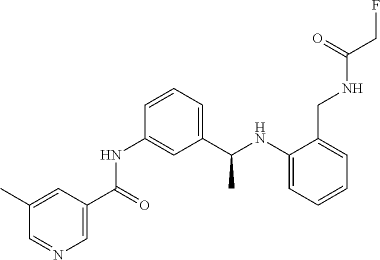 [SMILES: Cc1cncc(C(=O)Nc2cccc([C@H](C)Nc3ccccc3CNC(=O)CF)c2)c1] | −10 | | | −9 | 246 | 835 far | −9.76 | 69 | 822 far |
| pk10518 (S24) | 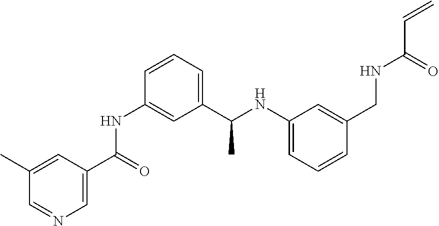 [SMILES: C=CC(=O)NCc1cccc(N[C@@H](C)c2cccc(NC(=O)c3cncc(C)c3)c2)c1] | −11.39 | | | −10.74 | 13 | 814 | −10.6 | 18.6 | 822 |
| pk10518p (S25) | 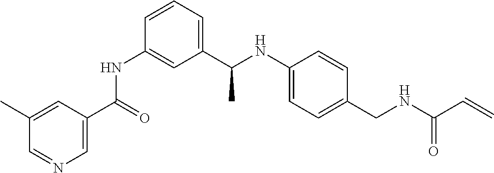 [SMILES: C=CC(=O)NCc1ccc(N[C@@H](C)c2cccc(NC(=O)c3cncc(C)c3)c2)cc1] | | | | −9.69 | 79 | 814 | −10.1 | 40.6 | 822 |
| pk10519 (S26 enantiomer) | 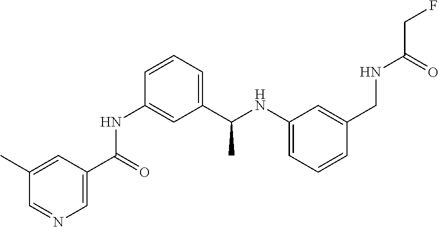 [SMILES: Cc1cncc(C(=O)Nc2cccc([C@H](C)Nc3cccc(CNC(=O)CF)c3)c2)c1] | −10.97 | | | | | | | | |
| pk10528 (S27) | 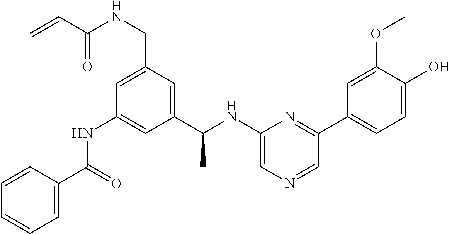 [SMILES: C=CC(=O)NCc1cc(NC(=O)c2ccccc2)cc([C@H](C)Nc2cncc(-c3ccc(O)c(OC)c3)n2)c1] | | | | −9.46 | 115 | 814 | −9.95 | 51 | 822 far |
| pk10533 (S34) | 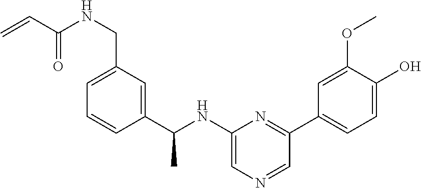 [SMILES: C=CC(=O)NCc1cccc([C@H](C)Nc2cncc(-c3ccc(O)c(OC)c3)n2)c1] | | | | −9.12 | 207 | 835, closer | −9.47 | | |
| pk10535 (S31) | 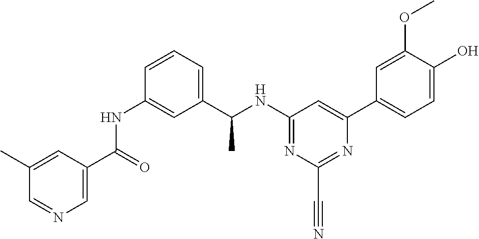 [SMILES: COc1cc(-c2cc(N[C@@H](C)c3cccc(NC(=O)c4cncc(C)c4)c3)nc(C#N)n2)ccc1O] | | | | −9.57 | 96 | very close 814 | −10 | 44 | very close 822 |
| pk10550 (S29) | 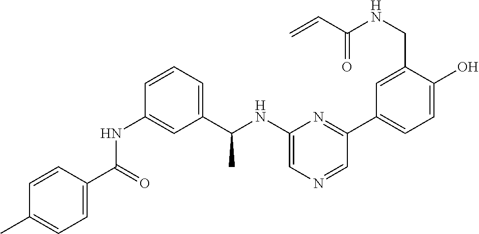 [SMILES: C=CC(=O)NCc1cc(-c2cncc(N[C@@H](C)c3cccc(NC(=O)c4ccc(C)cc4)c3)n2)ccc1O] | −12.67 | 514 pM | flipped away from CYS788 | −10.25 | 30.6 | 814 very close | −10.1 | 41.6 | 822 far |
| pk10559 (S33) | 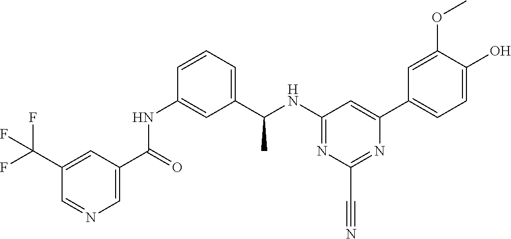 [SMILES: COc1cc(-c2cc(N[C@@H](C)c3cccc(NC(=O)c4cncc(C(F)(F)F)c4)c3)nc(C#N)n2)ccc1O] | | | | −10.04 | 44 | 814 very close | −10.5 | 19.4 | 822 very close |
| pk10560 (S28) | 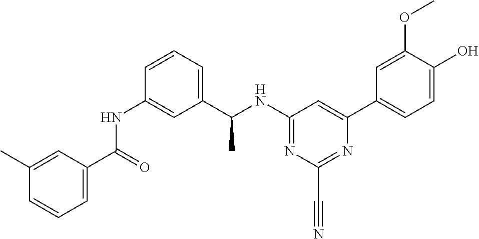 [SMILES: COc1cc(-c2cc(N[C@@H](C)c3cccc(NC(=O)c4cccc(C)c4)c3)nc(C#N)n2)ccc1O] | | | | −9.87 | 58 | 814 close | −10.02 | 44.8 | 822 far |
| pk10561 (S32) | 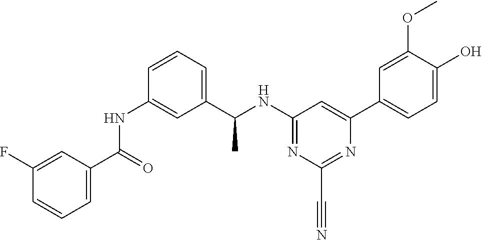 [SMILES: COc1cc(-c2cc(N[C@@H](C)c3cccc(NC(=O)c4cccc(F)c4)c3)nc(C#N)n2)ccc1O] | | | | −9.44 | | | −10.12 | | |
| pk10562 (S30) | 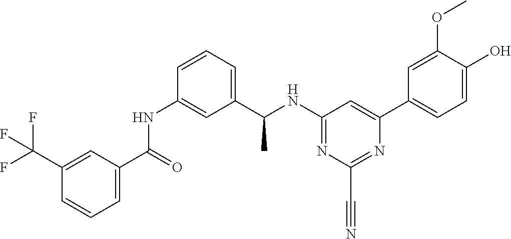 [SMILES: COc1cc(-c2cc(N[C@@H](C)c3cccc(NC(=O)c4cccc(C(F)(F)F)c4)c3)nc(C#N)n2)ccc1O] | −13.29 | | | −10.37 | 25 | 814 very close | −10.75 | 13.2 | 822 very close |

What is claimed is:

1. A method of treating pulmonary arterial hypertension (PAH) in a subject or a biological condition associated with PAH in a subject, and/or a method of preventing or reducing elevated pulmonary pressure in a subject, comprising: administering to the subject a therapeutically effective amount of a compound of Structure 1, a tautomer of the compound, a pharmaceutically acceptable salt of the compound, a pharmaceutically acceptable salt of the tautomer, or a mixture thereof, wherein Structure 1 has the following formula: 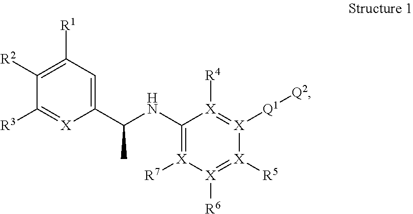
and wherein
X is independently selected from carbon or nitrogen;
R¹and R² may be the same or different and are independently selected from the group consisting of H, C, N, O, S, Cl, Br, F, I, —CN, —NO₂, —OH, —CH₃, —CF₃, —C—N—C— groups, —C—N—C(═O)— groups, substituted and unsubstituted amidinyl groups, substituted and unsubstituted guanidinyl groups, substituted and unsubstituted aryl groups, substituted and unsubstituted alkenyl groups, substituted and unsubstituted alkynyl groups, substituted and unsubstituted heterocyclyl groups, substituted and unsubstituted aminoalkyl groups;
R⁴, R⁵, R⁶, and R⁷, may be the same or different and are independently selected from the group consisting of absent, H, Cl, Br, F, I, —CN, —NO₂, —OH, —CH₃, —CF₃, —NH₂, —C≡N, —C═N groups, —C—N—C— groups, —C—N—C(═O)— groups, —C—N—C(═O)—C—F, —C—N—C(═O)—C═C, substituted and unsubstituted alkyl groups, substituted and unsubstituted aryl groups, substituted and unsubstituted heterocyclyl groups, alkoxy groups, aryloxy groups, substituted and unsubstituted heterocyclylalkyl groups, substituted and unsubstituted aminoalkyl groups, —C(═O)H, —C(═O)-alkyl groups, —C(═O)-aryl groups, —C(═O)O— alkyl groups, —C(═O)O-aryl groups, —C(═O)NH₂, substituted and unsubstituted alkyl group, substituted and unsubstituted aryl groups, —OH, substituted and unsubstituted alkoxy groups, substituted and unsubstituted aryloxy groups, substituted and unsubstituted heterocyclyl groups, —NHOH, —N(alkyl)OH groups, —N(aryl)OH groups, —N(alkyl)O-alkyl groups, —N(aryl)O-alkyl groups, —N(alkyl)O-aryl groups, and —N(aryl)O-aryl groups;
R³ is selected from the group consisting of: Q³, 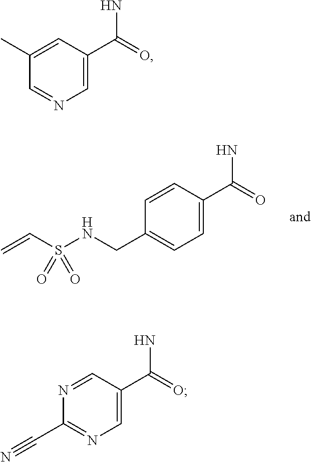
Q¹ and Q² are independently selected from the group consisting of a direct bond, —CH₃, —OH, —O—CH₃, —C—N—C(═O)—C═C, —C—N—C(═O)—C—F, and the following structures: 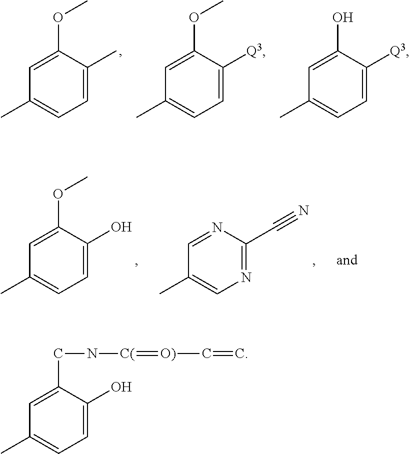
Q³ is selected from the group consisting of absent, a direct bond, H, C, Cl, Br, F, I, —CN, —NO₂, —CH₃, —CF₃, —NH₂, —C(═O)—, —C—N—R¹², —C≡N, —C—N—C groups, —C—N—C(═O)— groups, —C—N—C(═O)—C—F, —C—N—C(═O)—C═C, —C═N groups, —C(═O)— groups, —C(═O)—C— groups, —C(═O)—C═C, —CF₃, —C≡N, —C—N—C— groups, —C—N—C(═O)— groups, —C—N—C(═O)—C—F, —C—N—C(═O)—C═C, —OH, alkoxy groups, alkoxy groups, aryloxy groups, substituted and unsubstituted alkyl groups, substituted and unsubstituted aryl groups, substituted and unsubstituted heterocyclyl groups, —OH, alkoxy groups, and aryloxy groups, and combinations thereof; and
wherein the elevated pulmonary pressure in the subject is elevated compared to a healthy subject in a control population, and further wherein the reduced pulmonary pressure is reduced compared to the pulmonary pressure in the subject prior to the administering.

2-4. (canceled)

5. The method of claim 1, wherein the compound of Structure 1 is a compound of Structure

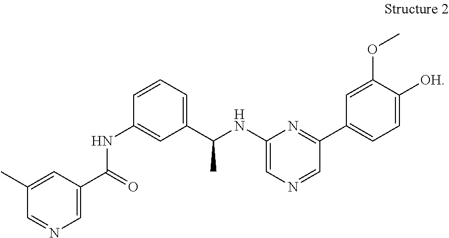 [SMILES: COc1cc(-c2cncc(N[C@@H](C)c3cccc(NC(=O)c4cncc(C)c4)c3)n2)ccc1O]

6. The method of claim 1, wherein the compound of Structure 1 is administered orally, intravenously, subcutaneously, transdermally, intraperitoneally, or by inhalation.

7. The method of claim 1, wherein the PAH is characterized by neointimal lesions or plexiform lesions, or both.

8. The method of claim 1, wherein the PAH, the biological condition associated with PAH, and/or wherein the PAH is secondary to one or more of the following indications: primary PAH, idiopathic PAH, heritable PAH, refractory PAH, BMPR2, ALK1, endoglin associated with hereditary hemorrhagic telangiectasia, endoglin not associated with hereditary hemorrhagic telangiectasia, drug-induced PAH, and toxin-induced PAH, PAH associated with systemic sclerosis, mixed connective tissue disease, HIV infection, hepatitis, portal hypertension, pulmonary hypertension, congenital heart disease, hypoxia, chronic hemolytic anemia, newborn persistent pulmonary hypertension, pulmonary veno-occlusive disease (PVOD), pulmonary capillary hemangiomatosis (PCH), left heart disease pulmonary hypertension, systolic dysfunction, diastolic dysfunction, valvular disease, lung disease, interstitial lung disease, pulmonary fibrosis, schistosomiasis, chronic obstructive pulmonary disease (COPD), sleep-disordered breathing, alveolar hypoventilation disorders, chronic exposure to high altitude, developmental abnormalities, chronic thromboembolic pulmonary hypertension (CTEPH), pulmonary hypertension with unclear multifactorial mechanisms, hematologic disorders, myeloproliferative disorders, splenectomy, systemic disorders, sarcoidosis, pulmonary Langerhans cell histiocytosis, lymphangioleimoyomatosis, neurofibromatosis, vasculitis, metabolic disorders, glycogen storage disease, Gaucher disease, thyroid disorders, tumoral obstruction, fibrosing mediastinitis, chronic renal failure on dialysis, abnormal: right ventricular systolic pressure (RVSP); pulmonary pressure; cardiac output; right ventricular (RV) hypertrophy and pulmonary arterial (PA) hypertrophy.

9-10. (canceled)

11. The method of claim 1, wherein the salt is a sulfate, phosphate, mesylate, bismesylate, tosylate, lactate, tartrate, malate, bis-acetate, citrate, or bishydrochloride salt.

12. The method of claim 1, wherein the compound of Structure 1, the tautomer of the compound, the pharmaceutically acceptable salt of the compound, the pharmaceutically acceptable salt of the tautomer, or the mixture thereof, is administered in combination with a second drug, and wherein the second drug is selected from the group consisting of prostanoids, endothelin antagonists, PDE5 inhibitors, cytoplasmic kinase inhibitors, receptor kinase inhibitors, guanylate cyclase stimulators, calcium channel blockers, beta blockers, angiotensin receptor antagonists, and ACE inhibitors.

13. (canceled)

14. The method of claim 1, wherein the therapeutically effective amount of a compound of Structure 1, the tautomer of the compound, the pharmaceutically acceptable salt of the compound, the pharmaceutically acceptable salt of the tautomer, or the mixture thereof, is not associated with adverse side effects, and wherein the adverse side effects comprise one or more of decreased lung function, increased or decreased systemic blood pressure, immunocompromised, bone marrow suppression, anemia, hypoxia, in the subject compared to the subject prior to the administering.

15. (canceled)

16. The method of claim 1, wherein the compound of Structure 1, the tautomer of the compound, the pharmaceutically acceptable salt of the compound, the pharmaceutically acceptable salt of the tautomer, or the mixture thereof, covalently interacts with a receptor tyrosine kinase (RTK), wherein the RTK is PDGFR or cKit or both, wherein the PDGFR is selected from the group consisting of PDGFR-α, PDGFR-β, PDGFR-αα, PDGFR-ββ, and PDGFR-αβ.

17-19. (canceled)

20. The method of any one of claims 1-19, wherein the compound of Structure 1, the tautomer of the compound, the pharmaceutically acceptable salt of the compound, the pharmaceutically acceptable salt of the tautomer, or the mixture thereof is administered in a total daily dosage from about 0.001 mg/kg to about 1 mg/kg by inhalation or oral.

21-24. (canceled)

25. The method of claim 1, wherein the compound of Structure 1 is a compound of Structure 3, 4, 5, 6, 7, 8, 9, 10, 11 or 12, as shown in Chart A, wherein R is H, F, CH₃, or CF₃, and/or wherein the compound of Structure 1 is a compound of Structure 13, 14, 15, 16, 17, 18, 19, 20, 21, 22, 23, 24, 25, 26, 27, 28, 29, 30, 31, 32, 33, or 34, as shown in Chart B.

26. (canceled)

27. The method of claim 1, wherein the reduction in pulmonary pressure is associated with an increase in one or more of RV function, and cardiac output in the subject compared to the subject prior to the administering and/or wherein the reduction in pulmonary pressure is associated with a decrease in one or more of RV hypertrophy, PA hypertrophy, RVSP, and sustained PA pressure in the subject compared to the subject prior to the administering.

28-29. (canceled)

30. The method of claim 27, wherein the reduction in pulmonary pressure is not associated with decreased lung function and increased systemic blood pressure in the subject compared to the subject prior to the administering and/or wherein the reduction in pulmonary pressure is a decrease in pulmonary arterial pressure in the subject compared to the subject prior to the administering.

31-36. (canceled)

37. A compound of Structure 1, a tautomer of the compound, a pharmaceutically acceptable salt of the compound, a pharmaceutically acceptable salt of the tautomer, or a mixture thereof, for treating one or more diseases associated with hyperproliferation, neoplasia, hyperplasia, dysplasia, angiogenesis, inflammation, immunological state, metabolism, pulmonary function, and cardiovascular function, wherein Structure 1 has the following formula: 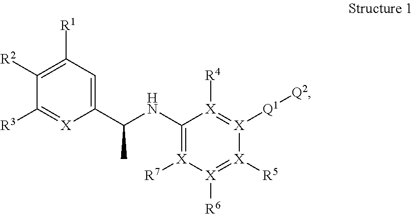
and wherein
X is independently selected from carbon or nitrogen;
R¹ and R² may be the same or different and are independently selected from the group consisting of H, C, N, O, S, Cl, Br, F, I, —CN, —NO₂, —OH, —CH₃, —CF₃, —C—N—C— groups, substituted and unsubstituted amidinyl groups, substituted and unsubstituted guanidinyl groups, substituted and unsubstituted aryl groups, substituted and unsubstituted alkenyl groups, substituted and unsubstituted alkynyl groups, substituted and unsubstituted heterocyclyl groups, substituted and unsubstituted aminoalkyl groups;
R⁴, R⁵, R⁶, and R⁷, may be the same or different and are independently selected from the group consisting of absent, H, Cl, Br, F, I, —CN, —NO₂, —OH, —CH₃, —CF₃, —NH₂, —C≡N, —C═N groups, —C—N—C— groups, —C—N—C(═O)— groups, —C—N—C(═O)—C—F, —C—N—C(═O)—C═C, substituted and unsubstituted alkyl groups, substituted and unsubstituted aryl groups, substituted and unsubstituted heterocyclyl groups, alkoxy groups, aryloxy groups, substituted and unsubstituted heterocyclylalkyl groups, substituted and unsubstituted aminoalkyl groups, —C(═O)H, —C(═O)-alkyl groups, —C(═O)-aryl groups, —C(═O)O-alkyl groups, —C(═O)O-aryl groups, —C(═O)NH₂, substituted and unsubstituted alkyl groups, substituted and unsubstituted aryl groups, —OH, substituted and unsubstituted alkoxy groups, substituted and unsubstituted aryloxy groups, substituted and unsubstituted heterocyclyl groups, —NHOH, —N(alkyl)OH groups, —N(aryl)OH groups, —N(alkyl)O-alkyl groups, —N(aryl)O-alkyl groups, —N(alkyl)O-aryl groups, and —N(aryl)O-aryl groups;
R³ is selected from the group consisting of: Q³, 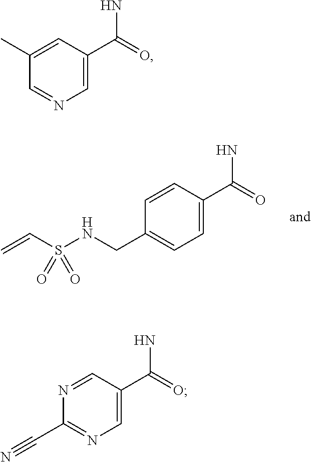
Q¹ and Q² are independently selected from the group consisting of a direct bond, —CH₃, —OH, —O—CH₃, —C—N—C(═O)—C═C, —C═N—C(═O)—C—F, and the following structures: 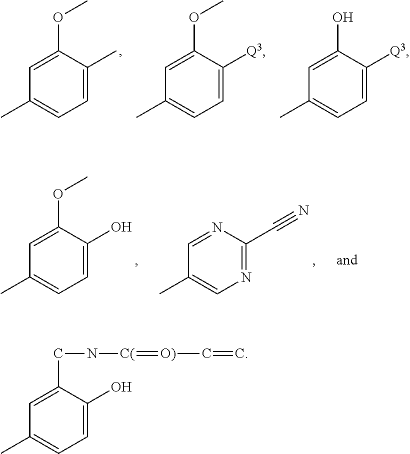
Q³ is selected from the group consisting of absent, a direct bond, H, C, Cl, Br, F, I, —CN, —NO₂, —CH₃, —CF₃, —NH₂, —C(═O)—, —C—N—R¹², —C≡N, —C—N—C groups, —C—N—C(═)— groups, —C—N—C(═O)—C—F, —C—N—C(═O)—C═C, —C═N groups, —C(═O)— groups, —C(═O)—C— groups, —C(═O)—C═C, —CF₃, —C≡N, —C—N—C— groups, —C—N—C(═O)— groups, —C—N—C(═O)—C—F, —C—N—C(═O)—C═C, —OH, alkoxy groups, alkoxy groups, aryloxy groups, substituted and unsubstituted alkyl groups, substituted and unsubstituted aryl groups, substituted and unsubstituted heterocyclyl groups, —OH, alkoxy groups, and aryloxy groups, and combinations thereof.

38-39. (canceled)

40. The compound of claim 37, wherein the compound of Structure 1 is a compound of

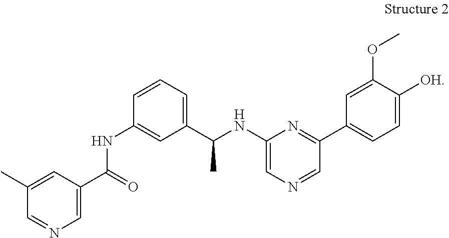 [SMILES: COc1cc(-c2cncc(N[C@@H](C)c3cccc(NC(=O)c4cncc(C)c4)c3)n2)ccc1O]

41. The compound of claim 37, wherein the compound of Structure 1 is administered orally, intravenously, subcutaneously, transdermally, intraperitoneally, or by inhalation.

42. The compound of claim 37, wherein the disease is selected from the group consisting of cancer, metastatic cancer, HIV, hepatitis, PAH, primary PAH, idiopathic PAH, heritable PAH, refractory PAH, BMPR2, ALK1, endoglin associated with hereditary hemorrhagic telangiectasia, endoglin not associated with hereditary hemorrhagic telangiectasia, drug-induced PAH, and toxin-induced PAH, PAH associated with systemic sclerosis, mixed connective tissue disease, pulmonary hypertension, congenital heart disease, hypoxia, chronic hemolytic anemia, newborn persistent pulmonary hypertension, pulmonary veno-occlusive disease (PVOD), pulmonary capillary hemangiomatosis (PCH), left heart disease pulmonary hypertension, systolic dysfunction, diastolic dysfunction, valvular disease, lung disease, interstitial lung disease, pulmonary fibrosis, schistosomiasis, COPD, sleep-disordered breathing, alveolar hypoventilation disorders, chronic exposure to high altitude, developmental abnormalities, chronic thromboembolic pulmonary hypertension (CTEPH), pulmonary hypertension with unclear multifactorial mechanisms, hematologic disorders, myeloproliferative disorders, splenectomy, systemic disorders, sarcoidosis, pulmonary Langerhans cell histiocytosis, lymphangioleimoyomatosis, neurofibromatosis, vasculitis, metabolic disorders, glycogen storage disease, Gaucher disease, thyroid disorders, tumoral obstruction, fibrosing mediastinitis, and chronic renal failure on dialysis.

43. The compound of claim 37, wherein the salt is a sulfate, phosphate, mesylate, bismesylate, tosylate, lactate, tartrate, malate, bis-acetate, citrate, or bishydrochloride salt.

44-47. (canceled)

48. The compound of claim 37, wherein the compound of Structure 1 is a compound of Structure 3, 4, 5, 6, 7, 8, 9, 10, 11 or 12, as shown in Chart A, wherein R is H, F, CH₃, or CF₃, and/or wherein the compound of Structure 1 is a compound of Structure 13, 14, 15, 16, 17, 18, 19, 20, 21, 22, 23, 24, 25, 26, 27, 28, 29, 30, 31, 32, 33 or 34, as shown in Chart B.

49. (canceled)

50. The method of claim 1, wherein the treatment results in one or more of improved exercise capacity, increased 6 minute walk distance, improved functional class, an improvement from class IV to class III, II or I, or an improvement from class III to class II or I, or an improvement form class II to class I, less shortness of breath, decreased hospitalization, decreased need for lung transplantation, decreased need for atrial septostomy, and increased longevity or overall survival.

51-52. (canceled)
